(19) United States
(12) Patent Application Publication
(10) Pub. No.: US 2022/0175816 A1
(43) Pub. Date: Jun. 9, 2022

(54) AIM2 INHIBITORS AND USES THEREOF

(71) Applicant: University of Massachusetts, Boston, MA (US)

(72) Inventors: Keitaro Fukuda, Worcester, MA (US); John E. Harris, Sterling, MA (US); Anastasia Khvorova, Westborough, MA (US); Kate Fitzgerald, Wellesley, MA (US)

(21) Appl. No.: 17/601,522

(22) PCT Filed: Apr. 16, 2020
(86) PCT No.: PCT/US20/28535

§ 371 Date: Oct. 5, 2021

Related U.S. Application Data
(60) Provisional application No. 62/972,831, filed on Feb. 11, 2020.
(60) Provisional application No. 62/835,861, filed on Apr. 18, 2019.

(51) Int. Cl. A61K 31/713; C12N 15/113

(52) U.S. Cl. CPC A61K 31/713; C12N 15/113; C12N 2310/14; C12N 2310/3515; C12N 2310/343; C12N 2310/346; C12N 2310/315

(57) ABSTRACT

Described herein are AIM2 inhibitors (e.g., inhibitory nucleic acids), vectors, cells (e.g., dendritic cells), and compositions comprising same, and methods of using same in the treatment of cancer (e.g., melanoma).

### CLAIM OF PRIORITY

This application claims the benefit of U.S. Provisional Patent Applications Ser. Nos. 62/835,861, filed on Apr. 18, 2019; and 62/972,831, filed on Feb. 11, 2020. The entire contents of the foregoing are hereby incorporated by reference.

### FEDERALLY SPONSORED RESEARCH OR DEVELOPMENT

This invention was made with Government support under Grant No. AR069114 and 0D020012 awarded by the National Institutes of Health. The Government has certain rights in the invention.

### TECHNICAL FIELD

Described herein are AIM2 inhibitors (e.g., inhibitory nucleic acids), vectors, cells (e.g., dendritic cells), and compositions comprising same, and methods of using same in the treatment of cancer (e.g., melanoma).

### BACKGROUND

Melanoma is an aggressive skin cancer with high mortality in those with advanced disease. However, melanoma is particularly immunogenic, which increases its susceptibility to immunotherapy. The advent of adoptive T cell therapy (ACT) and anti-PD-1 antibody (Ab) therapy has remarkably improved the prognosis of patients with stage IV melanoma. However, durable responses to these therapies are limited to 30-45% of patients (Goff et al., 2016; Ribas et al., 2016; Robert et al., 2015), representing a significant unmet need for patients who do not respond to current immunotherapies.

The advent of adoptive T cell therapy (ACT) and anti-programmed cell death protein 1 (PD-1) antibody has improved the prognosis of stage IV cancers (e.g., melanoma); however, durable responses to these therapies are limited to, e.g., with respect to melanoma, 30-45% of patients. Thus, there is a need to establish a combination immunotherapy that works also for the large number of patients who do not respond to current immunotherapies.

### SUMMARY

Described herein are double stranded RNA molecules, preferably between 15 and 35 bases in length, comprising a sense strand and an antisense strand, wherein the antisense strand comprises a region of complementarity that is substantially complementary to a nucleic acid sequence comprising nucleotides 362-380 of SEQ ID NO:46, nucleotides 662-681 of SEQ ID NO:48, nucleotides 714-732 of SEQ ID NO:46, nucleotides 1034-1051 of SEQ ID NO:48, or nucleotides 941-960 of SEQ ID NO: 48, optionally wherein the RNA molecule is modified. Also provided are single stranded molecules comprising either sense or antisense sequences provided herein.

In some embodiments, the sense strand comprises the sequence UUUGUAAAAGUUUUA (SEQ ID NO:29), GUUGAAUUAUAUGCA (SEQ ID NO:27), or GCUGAAAGCUAUAAA (SEQ ID NO:31), or differs by 1, 2, or 3 nucleotides. In some embodiments, the sense strand comprises the sequence (mU)#(mU)#(fU)(mG)(fU)(mA)(fA)(mA)(fA)(mG)(mU)(mU)(fU)#(mU)#(mA)-TegChol (SEQ ID NO:7), (mG)#(mU)#(fU)(mG)(fA)(mA)(fU)(mU)(fA)(mU)(mA)(mU)(fG)#(mC)#(mA)-TegChol (SEQ ID NO:3), or (mG)#(mC)#(fU)(mG)(fA)(mA)(fA)(mG)(fC)(mU)(mA)(mU)(fA)#(mA)#(mA)-TegChol (SEQ ID NO:17), wherein m is 2′-O-methyl, f is 2′-fluoro, # is a phosphorothioate bond, P is a 5′-phosphate, TegChol is 3′-Tetraethylene Glycol (Teg) Cholesterol Conjugate, and ( ) is a phosphodiester bond.

In some embodiments, the antisense strand comprises the sequence UAAAACUUUUACAAAGAAGA (SEQ ID NO:30), UGCAUAUAAUUCAACUUCUG (SEQ ID NO:28), UUUAUAGCUUUCAGCACCGU (SEQ ID NO:32), or differs by 1, 2, or 3 nucleotides. In some embodiments, the antisense strand comprises P(mU)#(fA)#(mA)(fA)(fA)(fC)(mU)(fU)(mU)(fU)(mA)(fC)(mA)#(fA)#(mA)#(fG)#(mA)#(mA)#(mG)#(fA) (SEQ ID NO:8), P(mU)#(fG)#(mC)(fA)(fU)(fA)(mU)(fA)(mA)(fU)(mU)(fC)(mA)#(fA)#(mC)#(fU)#(mU)#(mC)#(mU)#(fG) (SEQ ID NO:4), or P(mU)#(fU)#(mU)(fA)(fU)(fA)(mG)(fC)(mU)(fU)(mU)(fC)(mA)#(fG)#(mC)#(fA)#(mC)#(mC)#(mG)#(fU) (SEQ ID NO:18), wherein m is 2′-O-methyl, f is 2′-fluoro, # is a phosphorothioate bond, P is a 5′-phosphate, TegChol is 3′-Tetraethylene Glycol (Teg) Cholesterol Conjugate, and ( ) is a phosphodiester bond.

In some embodiments, the sense strand comprises the sequence UUUGUAAAAGUUUUA (SEQ ID NO:29), or differs by 1, 2, or 3, nucleotides, and the antisense strand comprises the sequence UAAAACUUUUACAAAGAAGA (SEQ ID NO:30), or differs by 1, 2, or 3 nucleotides. In some embodiments, the sense strand comprises the sequence (mU)#(mU)#(fU)(mG)(fU)(mA)(fA)(mA)(fA)(mG)(mU)(mU)(fU)#(mU)#(mA)-TegChol (SEQ ID NO:7) and the antisense strand comprises the sequence P(mU)#(fA)#(mA)(fA)(fA)(fC)(mU)(fU)(mU)(fU)(mA)(fC)(mA)#(fA)#(mA)#(fG)#(mA)#(mA)#(mG)#(fA) (SEQ ID NO:8), wherein m is 2′-O-methyl, f is 2′-fluoro, # is a phosphorothioate bond, P is a 5′-phosphate, TegChol is 3′-Tetraethylene Glycol (Teg) Cholesterol Conjugate, and ( ) is a phosphodiester bond.

In some embodiments, the sense strand comprises the sequence GUUGAAUUAUAUGCA (SEQ ID NO:27), or differs by 1, 2, or 3, nucleotides, and the antisense strand comprises the sequence UGCAUAUAAUUCAACUUCUG (SEQ ID NO:28), or differs by 1, 2, or 3 nucleotides. In some embodiments, the sense strand comprises the sequence (mG)#(mU)#(fU)(mG)(fA)(mA)(fU)(mU)(fA)(mU)(mA)(mU)(fG)#(mC)#(mA)-TegChol (SEQ ID NO:3) and the antisense strand comprises the sequence P(mU)#(fG)#(mC)(fA)(fU)(fA)(mU)(fA)(mA)(fU)(mU)(fC)(mA)#(fA)#(mC)#(fU)#(mU)#(mC)#(mU)#(fG) (SEQ ID NO:4), wherein m is 2′-O-methyl, f is 2′-fluoro, # is a phosphorothioate bond, P is a 5′-phosphate, TegChol is 3′-Tetraethylene Glycol (Teg) Cholesterol Conjugate, and ( ) is a phosphodiester bond.

In some embodiments, the sense strand comprises the sequence GCUGAAAGCUAUAAA (SEQ ID NO:31), or differs by 1, 2, or 3, nucleotides, and the antisense strand comprises the sequence UUUAUAGCUUUCAGCACCGU (SEQ ID NO:32), or differs by 1, 2, or 3 nucleotides. In some embodiments, the sense strand comprises the sequence (mG)#(mC)#(fU)(mG)(fA)(mA)(fA)(mG)(fC)(mU)(mA)(mU)(fA)#(mA)#(mA)-TegChol (SEQ ID NO:17) and the antisense strand comprises the sequence P(mU)#(fU)#(mU)(fA)(fU)(fA)(mG)(fC)(mU)(fU)(mU)(fC)(mA)#(fG)#(mC)#(fA)#(mC)#(mC)#(mG)#(fU) (SEQ ID NO:18), wherein m is 2′-O-methyl, f is 2′-fluoro, # is a phosphorothioate bond, P is a 5′-phosphate, TegChol is 3′-Tetraethylene Glycol (Teg) Cholesterol Conjugate, and ( ) is a phosphodiester bond.

Also provided herein are vectors comprising: (a) a nucleic acid molecule encoding an RNA, or (b) an RNA, as described herein.

Further, provided herein are cells comprising an RNA as described herein. In some embodiments, the cell is a dendritic cell.

In addition, provided herein are pharmaceutical compositions comprising the RNA molecules, vectors, or cells as described herein, and a pharmaceutically acceptable carrier.

Further, described herein are methods for reducing expression of AIM2 gene in a cell. The methods include: (a) introducing into the cell an RNA molecule as described herein, and (b) maintaining the cell produced in step (a) for a time sufficient to obtain degradation of the mRNA transcript of the AIM2 gene, thereby reducing expression of the AIM2 gene in the cell. In some embodiments, the cell is a dendritic cell.

Additionally, provided herein are methods for treating cancer in a subject in need thereof. The methods include administering to the subject a therapeutically effective amount of an RNA molecule, vector, cell, or pharmaceutical composition as described herein. Also provided are the RNA molecules, vectors, cells, or pharmaceutical compositions as described herein for use in a method of treating cancer.

In some embodiments, the RNA molecule, vector, cell, or pharmaceutical compositions is administered to, or formulated to be administered to, the subject intravenously, subcutaneously, or intratumorally.

In some embodiments, the cancer is melanoma.

In some embodiments, the method further comprises administering radiation or a cytotoxic agent to the subject.

In some embodiments, the method further comprises administering an immune checkpoint modulator to the subject. In some embodiments, the immune checkpoint modulator is an antagonist of programmed cell death protein 1 (PD-1). In some embodiments, the antagonist of PD1 is an antibody that specifically binds to PD-1. In some embodiments, the immune checkpoint modulator is an antagonist of cytotoxic T-lymphocyte-associated protein 4 (CTLA-4). In some embodiments, the antagonist of CTLA-4 is an antibody that specifically binds to CTLA-4. Also provided are pharmaceutical compositions comprising an immune checkpoint modulator and also comprising an RNA molecule, vector, or cell as described herein, and methods of use thereof for treating cancer in a subject in need thereof, or reducing expression of AIM2 gene in a cell.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-1L: AIM2 exerts an immunosuppressive effect in the melanoma microenvironment. (FIGS. 1A-1E) WT and Aim2^{-/-} mice were inoculated s.c. with B16F10 cells on day 0. On day 13, tissues were harvested. (FIG. 1A) Tumor growth over time (top; n=10 each). Sample photo of B16F10 tumor on day 13 (bottom). Bars, 10 mm. (FIGS. 1B-1D) Flow cytometry analysis of TILs. (FIG. 1B) The numbers of CD8⁺ and CD4⁺ T cells among 10⁴ live singlet cells (top). Percentage of FoxP3⁺ cells in CD4⁺ T cells and CD8/Treg ratio (bottom; n=10 each). (FIG. 1C) Representative contour plot for FoxP3 among CD4+ T cells. Numbers indicate % in the gate. (FIG. 1D) Percentages of IFN-γ⁺ and TNF-α⁺ among CD8⁺ T cells (n=10 each). (FIG. 1E) IFN-β protein levels within the tumor (n=7 each). (FIGS. 1F-1J) WT and Aim2^{-/-} mice were inoculated s.c. with YUMM1.7 cells on day 0. On day 17, tissues were harvested. (FIG. 1F) Tumor growth over time (top; n=11 each). Sample photo of YUMM 1.7 tumor on day 17 (bottom). Bars, 10 mm. (FIGS. 1G-1I) Flow cytometry analysis of TILs. (FIG. 1G) The numbers of CD8⁺ and CD4⁺ T cells among 10⁴ live singlet cells (top). Percentages of FoxP3⁺ cells in CD4⁺ T cells and CD8/Treg ratios (bottom; n=11 each). (FIG. 1H) Representative contour plot for FoxP3 among CD4⁺ T cells. Numbers indicate % in the gate. (FIG. 1I) Percentages of IFN-γ⁺ and TNF-α⁺ among CD8⁺ T cells (n=11 each). (J) IFN-β protein levels within the tumor (n=8 each). (FIG. 1K) The numbers of CD11c⁺ (left) and AIM2⁺ CD11c⁺ cells (middle), and the percentage of AIM2⁺ cells in CD11c⁺ cells (right) in high-powered field of primary lesions of human thin (n=15) and thick (n=16) melanoma. (FIG. 1L) Immunofluorescence microscopy of primary lesions of human thin and thick primary melanoma, visualized for CD11c, AIM2, and DAPI. Scale bar, 100 μtm. Mean±SEM combined from three independent experiments, analyzed by two-way ANOVA with Sidak's multiple-comparison test (A and F); mean±SEM combined from three (FIGS. 1B, 1D, 1G, and 1I), two (FIGS. 1E and 1J), or one (FIG. 1K) independent experiments, analyzed by Mann-Whitney's test. *p<0.05, **p<0.01, ****p<0.0001; NS, not significant. See also FIG. 9.

FIGS. 2A-2G: Vaccination with AIM2-deficient DC improves the efficacy of ACT through activation of STING-type I IFN signaling. (FIG. 2A) IFN-β or CXCL10 in the supernatants of indicated BMDCs stimulated with 0, 0.1, or 1 μg/mL B16F10 DNA for 4 (IFN-(β) or 10 h (CXCL10). (FIG. 2B) Immunoblotting for pTBK1, TBK1, pIRF3, IRF3, and vinculin in the lysates of indicated BMDCs stimulated with 0, 0.1, or 1 μg/mL B16F10 DNA for 4 h. (FIGS. 2C-2G) B16F10-bearing WT mice were treated with ACT alone or in combination with WT, Aim2^{-/-}, or Aim2^{-/-}Sting^{-/-}DC-gp100. On day 20 after PMEL transfer, tissues were harvested (n=9 each). (FIG. 2C) The therapy regimen scheme. (FIG. 2D) Tumor growth over time (left). Sample photo of B16F10 tumor on day 20 after PMEL transfer (right). (FIGS. 2E-2G) Flow cytometry analysis of TILs. (FIG. 2E) The numbers of PMELs, CD8⁺ T cells, and CD4⁺ T cells among 10⁴ live singlet cells, and PMEL/Treg ratio. (FIG. 2F) Representative contour plot for FoxP3 among CD4+T cells (left). Numbers indicate % in the gate. Percentages of FoxP3⁺ cells in CD4⁺ T cells (left). (FIG. 2G) Percentages of IFN-γ⁺ and TNF-α⁺ among PMELs. Mean±SEM combined from three independent experiments, analyzed by two-way ANOVA with Tukey's multiple-comparison test (FIG. 2D); mean±SEM combined from three independent experiments, analyzed by one-way ANOVA with Dunnett's (FIG. 2A) or Tukey's (FIGS. 2E-2G) multiple-comparison tests. *p<0.05, **p<0.01, ***p<0.001, ****p<0.0001; NS, not significant. See also FIG. 10.

FIGS. 3A-3G: Enhanced anti-melanoma immunity of vaccination with AIM2-deficient DC is dependent on the recognition of tumor-derived DNA and independent of reduced pyroptosis. (FIGS. 3A-3D) B16F10-bearing WT mice were treated with ACT with WT or Aim2^{-/-} DC-gp100 and intratumoral administration of DNase I or PBS. On day 20 after PMEL transfer, tissues were harvested (n=9 each). (FIG. 3A) Therapy regimen scheme. (FIG. 3B) Tumor growth over time (left). Sample photo of B16F10 tumor on day 20 after PMEL transfer (right). (FIGS. 3C and 3D) Flow cytometry analysis of TILs. (FIG. 3C) The number of PMELs and CDS⁺ T cells among 10⁴ live singlet cells. (FIG. 3D) The number of CD4⁺ T cells among 10⁴ live singlet cells, percentage of FoxP3⁺ cells in CD4⁺ T cells, and PMELs/Treg ratio. (FIG. 3E) Experimental scheme for analyzing DC vaccine infiltration in the tumor, TdLN, and spleen. B16F10-bearing CD45.1 congenic B6 mice were treated with ACT using PMELs (CD45.2) in combination with the intravenous administration of WT or Aim2^{-/-}DC-gp100 (CD45.2), and tissues were harvested on day 10 and day 20 after PMELs transfer. (FIG. 3F) Representative contour plot for CD45.2⁺ Thy1.1⁻CD11c⁺ MHCII⁺ DC-gp100 (DC vaccine) present at the tumor, TdLN, and spleen on day 20 after PMELs transfer. Numbers indicate % in the gate. (FIG. 3G) The absolute number of vaccinated DCs present in the tumor, TdLN, and spleen on day 10 (n=7 each) and 20 (n=8 each) after PMEL transfer. Mean±SEM combined from four independent experiments, analyzed by two-way ANOVA with Tukey's multiple comparison test (B) or one-way ANOVA with Tukey's multiple comparison test (FIGS. 3C and 3D); mean±SEM combined from three independent experiments, analyzed by Mann-Whitney's test (FIG. 3G). *p<0.05, **p<0.01, ***p<0.001, ****p<0.0001; NS, not significant. See also FIG. 11.

FIGS. 4A-4D: AIM2-deficient DC vaccination facilitates tumor antigen-specific CD8⁺ T-cell infiltration into the tumor via IFNAR signaling and CXCL10 production. (FIG. 4A) IFN-β or CXCL10 in the supernatants of indicated BMDCs stimulated with 0, 0.1, or 1 μg/mL B16F10 DNA for 4 (IFN-β) or 10 h (CXCL10). (FIGS. 4B-4D) B16F10-bearing WT mice were treated with ACT in combination with WT, Aim2^{-/-}, Aim2^{-/-}Ifnar^{-/-}or Aim2^{-/-}Cxcl10^{-/-} DC-gp100. On day 20 after PMEL transfer, tissues were harvested (n=10-11/group). (FIG. 4B) Tumor growth over time. (FIG. 4C and FIG. 4D) Flow cytometry analysis of TILs. (FIG. 4C) The numbers of PMELs and CD8⁺ T cells among 10⁴ live singlet cells. (FIG. 4D) The numbers of CD4⁺ T cells among 10⁴ live singlet cells, the percentages of FoxP3⁺ cells in CD4⁺ T cells, and PMEL/Treg ratios. Mean±SEM combined from three independent experiments, analyzed by two-way ANOVA with Tukey's multiple-comparison test (FIG. 4B); mean±SEM combined from three independent experiments, analyzed by one-way ANOVA with Dunnett's (FIG. 4A) or Tukey's (FIG. 4C and FIG. 4D) multiple-comparison test. *p<0.05, **p<0,01, ***p<0.001, ****p<0.0001; NS, not significant. See also FIG. 12.

FIGS. 5A-5G: Reduced IL-1β and IL-18 production by AIM2-deficient DC vaccination restricts Treg infiltration into the tumor. (FIG. 5A) IL-1β, IL-18, IFN-β, and CXCL10 in the supernatants of indicated BMDCs stimulated with 0, 0.1, or 1 μg/mL B16F10 DNA for 4 (IFN-β) or 10 h (IL-1β, IL-18, and CXCL10). (FIGS. 5B-5D) B16F10-bearing WT mice were treated with ACT in combination with WT, Aim2^{-/-}, or Il-1β^{-/-} DC-gp100. On day 20 after PMEL transfer, tissues were harvested (n=12-14/group). (FIG. 5B) Tumor growth over time. (FIG. 5C and FIG. 5D) Flow cytometry analysis of TILs. C, The numbers of PMELs and CD8⁺ T cells among 10⁴ live singlet cells. (FIG. 5D) The numbers of CD4⁺ T cells among 10⁴ live singlet cells, the percentages of FoxP3⁺ cells in CD4+T cells, and PMEL/Treg ratios. (FIGS. 5E-5G) B16F10-bearing WT mice were treated with ACT in combination with WT, Aim2^{-/-}, or Il-18^{-/-} DC-gp100. On day 20 after PMEL transfer, tissues were harvested (n=8-9/group). (FIG. 5E) Tumor growth over time. (FIG. 5F and FIG. 5G) Flow cytometry analysis of TILs. (FIG. 5F) The numbers of PMELs and CD8⁺ T cells among 10⁴ live singlet cells. (FIG. 5G) The numbers of CD4⁺ T cells among 10⁴ live singlet cells, the percentages of FoxP3⁺ cells in CD4⁺ T cells, and PMEL/Treg ratios. Mean±SEM combined from three independent experiments, analyzed by two-way ANOVA with Tukey's multiple-comparison test (B and E); mean±SEM combined from three independent experiments, analyzed by one-way ANOVA with Dunnett's (FIG. 5A) or Tukey's (FIGS. 5C, 5D, 5F, and G) multiple-comparison test. *p<0.05, **p<0.01, ***p<0.001, ****p<0.0001; NS, not significant. See also FIG. 13.

FIGS. 6A-6E: AIM2-silenced DC vaccine improves the efficacy of ACT against melanoma. (FIG. 6A) Immunoblotting for AIM2 and vinculin in the lysates of mock-, control siRNA-, or Aim2 siRNA (−4 or −9) transfected WT BMDCs 48 h after transfection. (FIG. 6B) Quantitative RT-PCR analysis of the Aim2 mRNA expression in mock-, control siRNA-, or Aim2 siRNA-transfected WT BMDCs 2, 10, and 22 days after transfection. (FIGS. 6C-6E) B16F10-bearing WT mice were treated with ACT in combination with control siRNA- or Aim2 siRNA-transfected WT DC-gp100. On day 20 after PMEL transfer, tissues were harvested (n=9 each). (FIG. 6C) Therapy regimen scheme. (FIG. 6D) Tumor growth over time (left). Sample photo of B16F10 tumor on day 20 after PMEL transfer (right). (FIG. 6E) Flow cytometry analysis of the numbers of PMELs, CD8⁺, and CD4⁺ T cells among 10⁴ live singlet cells, percentages of FoxP3⁺ cells in CD4⁺ T cells, and CD8/Treg ratios in the tumor. Mean±SEM combined from two independent experiments, analyzed by two-way ANOVA with Tukey's multiple-comparison test (FIG. 6D); mean±SEM combined from two independent experiments, analyzed by one-way ANOVA with Dunnett's (FIG. 6B) or Tukey's (FIG. 6E) multiple-comparison test. *p<0.05, **p<0.01, ***p<0.001; NS, not significant. See also FIG. 14.

FIGS. 7A-7E: AIM2-deficient DC vaccination potentiates the efficacy of anti-PD-1 immunotherapy. (FIGS. 7A-7E) WT mice were inoculated s.c. with B1 6F10 cells on day 0 and treated from the indicated time points by control IgG, PD-1 Ab, WT DC-gp100, Aim2^{-/-}DC-gp100, PD-1 Ab+WT DC-gp100, or PD-1 Ab+Aim2^{-/-} DC-gp100. On day 17, tissues were harvested (n=9-11/group). (FIG. 7A) Therapy regimen scheme. (FIG. 7B) Tumor growth over time. (FIGS. 7C-7E) Flow cytometry analysis of TILs. (FIG. 7C) The numbers of CD8⁺ and CD4⁺ T cells among 10⁴ live singlet cells. (FIG. 7D) The percentages of FoxP3⁺ cells in CD4⁺ T cells and CD8/Treg ratios. (FIG. 7E) The percentages of IFN-γ⁺ among CD8⁺ T cells. Mean±SEM combined from three independent experiments, analyzed by two-way ANOVA with Tukey's multiple-comparison test (FIG. 7B), or one-way ANOVA with Dunnett's multiple-comparison test (FIGS. 7C-7E). *p<0.05, **p<0.01, ***p<0.001, ****p<0.0001. See also FIG. 15.

FIGS. 8A-8E: siRNA targeting of AIM2 in human monocyte derived-DCs results in increased activation similar to mouse BMDCs. (FIG. 8A) Immunoblotting for AIM2 and vinculin in the lysates of Control siRNA- or Aim2 siRNA (−2 or −4)-transfected monocyte derived-DCs (MoDCs) 48 h after transfection. (FIG. 8B) Immunoblotting for AIM2 and vinculin in the lysates of non-primed or LPS-primed human MoDCs. (FIG. 8C) Immunoblotting for pTBK1, TBK1, pIRF3, IRF3, and vinculin in the lysates of indicated control siRNA- or Aim2 siRNA-transfected LPS-primed MoDCs stimulated with 0 or 1 μg/mL melanoma DNA for 8 h. (FIG. 8D) IFN-β, CXCL10, (FIG. 8E) IL-1β, and IL-18 in the supernatants of indicated LPS-primed human MoDCs (n=6) stimulated with 0 or 1 μg/mL human melanoma-derived DNA for 12 h. Data are mean±SEM, analyzed by Friedman tests with Dunn's multiple comparison test (FIG. 5D and FIG. 8E). *p<0.05, **p<0.01; NS, not significant.

FIGS. 9A-9F: Effects of host AIM2 deficiency on TdLN and spleen in B16F10 and YUMM1.7 model. (FIG. 9A) Gating strategy and representative flow cytometry plots for the assessment of CD4⁺ T, CD8⁺ T, Tregs, IFN-γ⁺ CD8⁺ T, TNF-α⁺ CD8⁺ T, PMELs, MAC, and DC in B16F10 melanoma. (FIG. 9B and FIG. 9C) Flow cytometry analysis of the numbers of MACs and DCs among 10⁵ live singlet cells in the tumor (FIG. 9B) and flow cytometry analysis of the numbers of CD8⁺ and CD4⁺ T cells among 10⁵ live singlet cells, percentages of FoxP3⁺ cells in CD4⁺ T cells, and CD8/Treg ratios in the TdLN and spleen (FIG. 9C) of WT and Aim2^{-/-} mice 13 days after B16F10 subcutaneous inoculation (n=10 each). (FIG. 9D and FIG. 9E) Flow cytometry analysis of the numbers of MACs and DCs among 10⁵ live singlet cells in the tumor (FIG. 9D) and flow cytometry analysis of the numbers of CD8⁺ and CD4⁺ T cells among 10⁵ live singlet cells, percentages of FoxP3⁺ cells in CD4⁺ T cells, and CD8/Treg ratios in the TdLN and spleen (FIG. 9E) of WT and Aim2^{-/-}mice 17 days after YUMM1.7 melanoma inoculation (n=11 each). (FIG. 9F) The numbers of CD11c⁺ (left) and AIM2⁺ CD11c⁺ cells (middle), and the percentage of AIM2⁺ cells in the CD11c⁺ gate (right) in high-powered field of primary lesions of non-metastatic (stage I and II, n=21) and metastatic (stage III and IV, n=10) melanoma. Mean±SEM combined from three (FIGS. 9B-9E) or one (FIG. 9F) independent experiments, analyzed by Mann-Whitney's test. *p<0.05; NS, not significant.

FIGS. 10A-10F: The effect of AIM2-deficient DC vaccine with ACT on tumor, TdLN, and spleen in the B1 6F10 model. (FIG. 10A) Quantitative RT-PCR analysis of Ifnb, Ifna, Cxcl10, and Cxcl9 mRNA expression in WT, Aim2^{-/-}, Aim2^{-/-} Sting^{-/-}, and Sting^{-/-} BMDCs stimulated with 0, 0.1, or 1 μg/mL B16F10-derived DNA for 4 h, presented in arbitrary units (A.U.), relative to Actb (encoding β-actin) expression. (FIG. 10B) Experimental scheme for analyzing DC vaccine infiltration in the tumor, TdLN, and spleen. B16F10-bearing CD45.1 congenic B6 mice were treated with ACT using PMELs (CD45.2) in combination with the intravenous administration of WT or Aim2^{-/-}DC-gp100 (CD45.2), and tissues were harvested 1.5 days after PMELs transfer. (FIG. 10C) The absolute number of transferred DCs present in the tumor, TdLN, and spleen (n=8 each). (FIGS. 10D-10F) Flow cytometry analysis of the numbers of MACs and DCs among 10⁵ live singlet cells in the tumor (FIG. 10D) and flow cytometry analysis of the numbers of PMELs, CD8⁺ T cells (FIG. 10E), and CD4⁺ T cells among 10⁵ live singlet cells, and percentages of FoxP3⁺ cells in CD4⁺ T cells (FIG. 10F) in the TdLN and spleen of B16F10-bearing WT mice treated with ACT in combination with WT, Aim2^{-/-}, or Aim2^{-/-}Sting^{-/-} DC-gp100 (n=9 each). Mean±SEM combined from three independent experiments, analyzed by one-way ANOVA with Dunnett's multiple-comparison test (FIG. 10A), Mann-Whitney's test (FIG. 10C), or one-way ANOVA with Tukey's multiple-comparison test (FIGS. 10D-10F). *p<0.05, **p<0.01, ***p<0.001, ****p<0.0001.

FIGS. 11A-11B: The role of DNA sensing in AIM2-deficient DC vaccine with ACT on TdLN and spleen in the B 1 6F10 model. (FIG. 11A and FIG. 11B) Flow cytometry analysis of the numbers of PMELs, total CD8⁺ T cells (FIG. 11A), and CD4⁺ T cells among 10⁵ live singlet cells and percentages of FoxP3⁺ cells in CD4⁺ T cells (FIG. 11B) in the TdLN and spleen of B16F10-bearing WT mice treated with ACT in combination with WT or Aim2^{-/-} DC-gp100 and intratumoral administration of DNase I or PBS (n=9/group). Mean±SEM combined from four (FIG. 11A and FIG. 11B) independent experiments, analyzed by one-way ANOVA with Tukey's multiple-comparison test. *p<0.05, **p<0.01.

FIGS. 12A-12B: The effect of AIM2-IFNAR and AIM2-CXCL10 double-deficient DC vaccination with ACT on TdLN and spleen in the B16F10 model. (FIG. 12A and FIG. 12B) Flow cytometry analysis of the numbers of PMELs, total CD8⁺ T cells (FIG. 12A), CD4⁺ T cells among 10⁵ live singlet cells, and percentages of FoxP3⁺ cells in CD4⁺ T cells (FIG. 12B) in the TdLN and spleen of B16F10-bearing WT mice treated with ACT in combination with WT, Aim2 ^{-/-}, Aim2^{-/-}Ifnar^{-/-} or Aim2^{-/-}Cxcl10^{-/-} DC-gp100 (n=10-11/group). Mean±SEM combined from three (FIG. 12A and FIG. 12B) independent experiments, analyzed by one-way ANOVA with Tukey's multiple-comparison test. *p<0.05, ***p<0.001.

FIGS. 13A-13D: Effect of IL-1β- and IL-18-deficient DC vaccination with ACT on TdLN and spleen in the B16F10 model. (FIG. 13A and FIG. 13B) Flow cytometry analysis of the numbers of PMELs, total CD8⁺ T cells (FIG. 13A), CD4⁺ T cells among 10⁵ live singlet cells, and percentages of FoxP3⁺ cells in CD4⁺ T cells (FIG. 13B) in the TdLN and spleen of B16F10-bearing WT mice treated with ACT in combination with WT, Aim2^{-/-}, or Il-1β^{-/-} DC-gp100 (n=12-14/group). (FIG. 13C and FIG. 13D) Flow cytometry analysis of the numbers of PMELs, total CD8⁺ T cells (FIG. 13C), CD4⁺ T cells among 10⁵ live singlet cells, and percentages of FoxP3⁺ cells in CD4⁺ T cells (FIG. 13D) in the TdLN and spleen of B16F10-bearing WT mice treated with ACT in combination with WT, Aim2^{-/-}, or Il-18^{-/-} DC-gp100 (n=8-9/group). Mean±SEM combined from three (FIGS. 13A-13D) independent experiments, analyzed by one-way ANOVA with Tukey's multiple-comparison test. *p<0.05, ***p<0.001.

FIGS. 14A-14C: Effect of control siRNA- and Aim2 siRNA-transfected WT DC vaccine with ACT on TdLN and spleen in the B16F10 model. (FIG. 14A) Quantitative RT-PCR analysis of the Aim2 mRNA expression in Mock- or Aim2 siRNA-transfected WT BMDCs 3 days after transfection. Arrows indicate Aim2 siRNA that were selected as Aim2 siRNA for in vitro and in vivo study. FIG. 14B and FIG. 14C) Flow cytometry analysis of the numbers of PMELs, CD8⁺ T cells (FIG. 14B), and CD4⁺ T cells among 10⁵ live singlet cells and percentages of FoxP3⁺ cells in CD4⁺ T cells (FIG. 14C) in the TdLN and spleen of B16F10-bearing WT mice treated with ACT in combination with control siRNA or Aim2 siRNA-transfected DC-gp100 (n=9 each). Mean±SEM combined from five independent experiments, analyzed by one-way ANOVA with Dunnett's multiple comparison test (FIG. 14A) and two (FIG. 14B and FIG. 14C) independent experiments, analyzed by one-way ANOVA with Tukey's multiple-comparison test. *p<0.05, **p<0.01, ***p<0.001.

FIGS. 15A-15C: Effect of AIM2-deficient DC vaccine with anti-PD-1 immunotherapy on TdLN and spleen in the B16F10 model. (FIG. 15A) Flow cytometry analysis of the percentage of TNF-α⁺ among CD8⁺ T cells infiltrated in the tumor. (FIG. 14B and FIG. 14C) Flow cytometry analysis of the numbers of CD8⁺ and CD4⁺ T cells among 10⁵ live singlet cells and percentages of FoxP3⁺ cells in CD4⁺ T cells in the TdLN (FIG. 15B) and spleen (FIG. 15C) of B16F10-bearing wild-type (WT) mice treated with Control IgG, PD-1 Ab, WT DC-gp100, or Aim2^{-/-} DC-gp100 (n=9-11/group). Mean±SEM combined from four (FIGS. 15A-15C) independent experiments, analyzed by one-way ANOVA with Dunnett's multiple-comparison test.

FIG. 16 depicts exemplary Aim2 siRNA sequences. SEQ ID NOs:1-26 from top to bottom, respectively.

### DETAILED DESCRIPTION

Growing evidence reveals that the success of immunotherapy strongly correlates with the numbers of tumor-infiltrating CD8⁺ T cells prior to therapy. A melanoma infiltrated by a large number of CD8⁺ T cells, referred to as a “hot tumor” to due to the amount of inflammation present, responds well to immunotherapies, while those infiltrated by few CD8⁺ T cells, referred to as a “cold tumor”, typically shows a poor response. The infiltration of CD8⁺ T cells into the tumor is facilitated by the recognition of tumor-derived DNA by the cytosolic cGAS-STING signaling pathway in tumor-infiltrating dendritic cells (TIDCs). This leads to the production of type I interferon (IFN) by TIDCs and promotes their migration to the tumor-draining lymph node. There they prime tumor antigen-specific T cells and induces their homing to the tumor. In this setting, STING agonists have been approved for use as an adjuvant therapy to increase the efficacy of PD-1 Ab treatment in patients with metastatic melanoma.

While the importance of cGAS-STING pathway signaling in TIDCs has been well established, tumor-derived cytosolic DNA can also be recognized by AIM2. AIM2 was initially identified as a gene that was lost in melanoma and other cancers. Despite its name, the function of AIM2 in the melanoma microenvironment is unknown. AIM2 is a cytosolic dsDNA binding protein that forms a caspase-1 activating inflammasome complex, resulting in proteolytic processing of the inflammatory cytokines IL-1β and IL-18 and the pore-forming protein gasdermin D, which elicits a lytic form of cell death called pyroptosis. IL-1β expression positively correlates with melanoma thickness, suggesting that the cytokine promotes tumor growth. Notably, most melanoma cells silence expression of one or more inflammasome components and do not produce IL-1β by themselves but instead induce IL-1β production from tumor-associated macrophages by releasing endogenous danger signals. IL-18 also belongs to the IL-1 family of cytokines and activates the MyD88-NF-κB signaling pathway; however, its effect on melanoma growth is nuanced. Treatment with IL-18 has been reported to suppresses melanoma growth and metastasis, but also accelerate melanoma growth by accumulating monocytic myeloid-derived suppressor cells in the melanoma microenvironment.

Existing immunotherapies for melanoma have limited efficacy when the tumor lacks sufficient infiltration by CD8+T cells, a condition known as a “cold tumor”. Strategies to activate TIDCs and promote T cell recruitment through treatment with STING agonists are currently being tested in clinical trials. Combining STING agonists with PD-1 Ab treatment has been considered to improve outcomes for “cold” melanomas. However, 48% of these tumors have aberrant activation of WNT/β-catenin signaling and lack CD103+TIDCs, and therefore, STING agonists, which stimulate the function of TIDC, may not be effective in “cold” tumors. In contrast, intra-tumoral injection of CD103+DCs reversed the resistance of melanoma with activated WNT/β-catenin signaling to ACT and anti-PD-1 Ab treatment. These results suggest that a treatment strategy that increases TIDC and also activates the STING-type I IFN pathway would be optimal for combined therapy with ACT and anti-PD-1 Ab.

This disclosure is based, in part, on the finding that vaccination using Aim2^{-/-} bone marrow-derived dendritic cells (BMDCs) provides an alternate approach to enhance immunotherapy, which may achieve therapeutic efficacy even for patients with cold tumors (see Examples). The Examples below show that, in contrast to STING, AIM2 exerts an immunosuppressive effect within the melanoma microenvironment and AIM2-deficient dendritic cell (Aim2^{-/-} DC) vaccination improves the efficacy of both adoptive T-cell therapy (ACT) and anti-PD-1 immunotherapy for “cold tumors”. Without being bound by any particular theory, this effect depends on tumor-derived DNA that activates STING-dependent type I IFN secretion and subsequent production of CXCL10 to recruit CD8⁺ T cells. In addition, loss of AIM2-dependent IL-1β and IL-18 processing further enhanced the treatment response by limiting the recruitment of T regulatory cells. Thus, targeting AIM2 in tumor-infiltrating DCs is a new treatment strategy for patients with cancer, such as advanced melanoma. These data support using vaccination with Aim22^{-/-} DCs as an adjuvant to ACT therapy or treatment with PD-1 antibodies.

### AIM2

AIM2 (also known as “absent in melanoma 2” and “interferon-inducible protein AIM2), is a protein that in humans is encoded by the AIM2 gene. AIM2 is involved in the innate immune response and recognizes cytosolic double-stranded DNA. AIM2 is a component of the AIM2 inflammasome, which produces mature IL-1β and IL-18, as well as induces a lytic form of cell death called pyroptosis. AIM2 has been reported to suppress the cGAS-STING-type I IFN signaling axis in bone marrow derived dendritic cells (BMDCs) and macrophages (BMDMs) in response to tumor-derived cytosolic DNA in vitro.

An exemplary amino acid sequence of human AIM2 is shown below:

| (GenBank Accession No. NP_004824; SEQ ID NO: 45) | |
|---|---|
| 1 | meskykeill ltgldnitde eldrfkffls defniatgkl htanriqvat lmignagavs |
| 61 | avmktirifq klnymllakr lqeekekvdk qyksvtkpkp lsqaemspaa saairndvak |
| 121 | qraapkvsph vkpeqkqmva qqesiregfq krclpvmvlk akkpftfetq egkqemfhat |
| 181 | vatekefffv kvfntllkdk fipkriiiia ryyrhsgfle vnsasrvlda esdqkvnvpl |
| 241 | niirkagetp kintlqtqpl gtivnglfvv qkvtekkkni lfdlsdntgk mevlgvrned |
| 301 | tmkckegdkv rltfftlskn geklqltsgv hstikvikak kkt |

An exemplary nucleic acid sequence of human AIM2 mRNA is shown below:

| (GenBank Accession No. NM_004833; SEQ ID NO: 46) | |
|---|---|
| 1 | agaagtgtca gagtctttgt agctttgaaa gtcacctagg ttatttgggc atgctctcct |
| 61 | gagtcctctg ctagttaagc tctctgaaaa gaaggtggca gacccggttt gctgatcgcc |
| 121 | ccagggatca ggaggctgat cccaaagttg tcagatggag agtaaataca aggagatact |
| 181 | cttgctaaca ggcctggata acatcactga tgaggaactg gataggttta agttctttct |
| 241 | ttcagacgag tttaatattg ccacaggcaa actacatact gcaaacagaa tacaagtagc |
| 301 | taccttgatg attcaaaatg ctggggcggt gtctgcagtg atgaagacca ttcgtatttt |
| 361 | tcagaagttg aattatatgc ttttggcaaa acgtcttcag gaggagaagg agaaagttga |
| 421 | taagcaatac aaatcggtaa caaaaccaaa gccactaagt caagctgaaa tgagtcctgc |
| 481 | tgcatctgca gccatcagaa atgatgtcgc aaagcaacgt gctgcaccaa aagtctctcc |
| 541 | tcatgttaag cctgaacaga aacagatggt ggcccagcag gaatctatca gagaagggtt |
| 601 | tcagaagcgc tgtttgccag ttatggtact gaaagcaaag aagcccttca cgtttgagac |
| 661 | ccaagaaggc aagcaggaga tgtttcatgc tacagtggct acagaaaagg aattcttctt |
| 721 | tgtaaaagtt tttaatacac tgctgaaaga taaattcatt ccaaagagaa taattataat |
| 781 | agcaagatat tatcggcaca gtggtttctt agaggtaaat agcgcctcac gtgtgttaga |
| 841 | tgctgaatct gaccaaaagg ttaatgtccc gctgaacatt atcagaaaag ctggtgaaac |
| 901 | cccgaagatc aacacgcttc aaactcagcc ccttggaaca attgtgaatg gtttgtttgt |
| 961 | agtccagaag gtaacagaaa agaagaaaaa catattattt gacctaagtg acaacactgg |
| 1021 | gaaaatggaa gtactggggg ttagaaacga ggacacaatg aaatgtaagg aaggagataa |
| 1081 | ggttcgactt acattcttca cactgtcaaa aaatggagaa aaactacagc tgacatctgg |
| 1141 | agttcatagc accataaagg ttattaaggc caaaaaaaaa acatagagaa gtaaaaagga |
| 1201 | ccaattcaag ccaactggtc taagcagcat ttaattgaag aatatgtgat acagcctctt |
| 1261 | caatcagatt gtaagttacc tgaaagctgc agttcacagg ctcctctctc caccaaatta |
| 1321 | ggatagaata attgctggat aaacaaattc agaatatcaa cagatgatca caataaacat |
| 1381 | ctgtttctca ttca |

An exemplary amino acid sequence of mouse AIM2 is shown below:

| (GenBank Accession No. NP_001013801; SEQ ID NO: 47) | |
|---|---|
| 1 | meseyremll ltgldhitee elkrfkyfal tefgiarstl dvadrtelad hliqsagaas |
| 61 | avtkainifq klnymhiana leekkkeaer klmtntkkrg tqkvenrsqa encsaasatr |
| 121 | sdndfkeqaa tevcpqakpq kkgmvaeqea iredlqkdpl vvtvlkainp fecetqegrq |
| 181 | eifhatvate tdfffvkvln aqfkdkfipk rtikisnylw hsnfmevtss svvvdvesnh |
| 241 | evpnnvvkra retprisklk iqpcgtivng lfkvqkitee kdrvlygihd ktgtmevlvl |
| 301 | gnpsktkcee gdkirltffe vskngvkiql ksgpcsffkv ikaakpktdm ksve |

An exemplary nucleic acid sequence of mouse AIM2 mRNA is shown below:

| (GenBank Accession No. NM_001013779; SEQ ID NO: 48) | |
|---|---|
| 1 | ttcctgtcct gtctgccgcc atgcttcctt aactagctgc taggtttttt ccttgtcgtg |
| 61 | atgaaatcca ccctcatgga cctacactac cgaactggac tgctggtata ttcatgaagt |
| 121 | gcttatgagt ggatcgagca gcccctatgg attcctgtga acagaactgc tgatttacta |
| 181 | acaacgcaga tggaagttgc ttcaaagaac aacttctgaa caggtattgt tgcccattct |
| 241 | gtgaataata caaaggcagt gggaacaaga cagtacagag gacttgattc aggagacttg |
| 301 | aggtctggcc gcatagtcat cctttagaag ctgggtggcg tcaggaagtt ttcctttttc |
| 361 | tcaatgtaaa gtgaagaaaa aaaatccagt gtttctcaac tgtactgcta ttcctattta |
| 421 | gctattgtat ctaggctgat cctgggactg tgagatggag agtgagtacc gggaaatgct |
| 481 | gttgttgacc ggcctggacc acatcacgga ggaagaactg aaacggttca agtactttgc |
| 541 | tttgactgag tttcagattg ccaggagcac actcgacgtg gcagatagga cagagttagc |
| 601 | tgaccacctg attcaaagtg caggtgcggc gtctgcagtg accaaggcca ttaatatttt |
| 661 | ccagaagttg aattatatgc atattgcaaa tgctcttgaa gagaaaaaga aagaagctga |
| 721 | acgtaaactc atgaccaata caaagaagag aggaacacag aaggtagaaa atagaagtca |
| 781 | agctgaaaac tgctctgctg cctctgccac ccgcagtgac aatgacttta aggaacaggc |
| 841 | tgctacagaa gtctgtcctc aagctaagcc tcagaagaaa cagatggtgg cagaacagga |
| 901 | agccatcaga gaagatttac agaaagatcc acttgttgtc acggtgctga aagctataaa |
| 961 | tccctttgag tgtgagactc aggaaggaag acaagagata tttcatgcaa cagtggccac |
| 1021 | ggagacagat tttttctttg taaaagtttt aaacgcacag tttaaagata aatttatccc |
| 1081 | aaagaggaca attaaaatat caaactacct ttggcacagt aacttcatgg aggtcaccag |
| 1141 | ttcctcagtt gtggttgatg ttgaatctaa ccacgaagtc ccaaataacg ttgttaagag |
| 1201 | agccagggaa actcccagga ttagtaaact gaagattcag ccatgtggaa caattgtgaa |
| 1261 | tgggctgttt aaagtccaga agataacaga ggaaaaagat agagtactgt atggtataca |
| 1321 | tgataaaaca gggacaatgg aggtgttggt gctgggaaac ccaagcaaaa caaagtgcga |
| 1381 | ggaaggagac aagattagac tcacgttctt tgaggtgtca aaaaatggag tgaaaattca |
| 1441 | gttgaaatct ggaccttgta gcttttttaa ggttattaag gctgcaaagc caaaaactga |
| 1501 | catgaaaagt gtggagtgaa gtcacctcat ttgaaaaacc ttttcctgaa gaatcctgat |
| 1561 | gctgctcctt gaactagact gaactacctg aggatagcat tttacaacct catcatcata |
| 1621 | ttgtattact tagaaaagga caaatactca aaaaacatct gaaaaatata tgtaaactta |
| 1681 | ttattaatta agttattaag actgcccaac ctggggatcc atcctatata caaccaccaa |
| 1741 | acccagacac tattgcatat gccagcaaga ttttgctgac aggatcctga tatagctctc |
| 1801 | tcttgtgagg ctctgccagt gactgacaag tacagaagca gatgctcaca gtcatctatt |
| 1861 | ggatggaaca cagggcccct aataaaggag ctagagaaag tacccaagca gcaagtggtc |
| 1921 | tgcaacgcta taggaggaac aacaacatga actaaccagt accccccaga actgtgtctc |
| 1981 | cagttgcata tgtagcagaa gatggcctgg ccggtcatca atgggaggag aggcccttgg |
| 2041 | tcttgcaaag atcatatgcc ccagtacagg ggaatgccag ggccaggcag caggagtgga |
| 2101 | tgtgggtggg ttggggagtg tgtgtggggg gtgttatagg ggactttcgg gatagcattt |
| 2161 | gaaatgtaaa tgaagaaaat atctaataaa attgttgctt tgtctaaggt ttgagatatc |
| 2221 | attcttctct acatagacac tgagggtata agtatggcgg gattgcagat gtgacagcag |
| 2281 | ggccttgtcg gagagacgcc tgtgggtgat agagaagatt ggtgatatat aattttttaa |
| 2341 | tttaaaaatt ttaaatttcc ttttggggag gaggttacag gtggaggagg gtgggtatga |
| 2401 | tagtactaag aaatcagtga tattggggta tgtgatgtga aattccctag cactcaataa |
| 2461 | aagaattatg tttttaaaaa gaaagattgt tgataaataa ataaatatga ttttactcat |
| 2521 | gattcagaaa gttagaaaaa a |

### AIM2 Inhibitors

The methods and compositions described herein can include inhibitors of AIM2. In some embodiments, the AIM2 inhibitor comprises a small molecule inhibitor of AIM2. In some embodiments, the AIM2 inhibitor comprises a polypeptide inhibitor of AIM2, e.g., an antibody or antigen-binding fragment thereof. In some embodiments, the AIM2 inhibitor comprises an inhibitory nucleic acid, e.g., an antisense oligonucleotide, a small interfering RNA (siRNA), a small hairpin RNA (shRNA), a molecule comprising modified base(s), a locked nucleic acid molecule (LNA molecule), a peptide nucleic acid molecule (PNA molecule), and other oligomeric compounds oligonucleotide mimetics that hybridize to at least a portion of the target nucleic acid and inhibit its function.

### Inhibitory Nucleic Acids

Inhibitory nucleic acids useful in the present methods and compositions include antisense oligonucleotides, ribozymes, siRNA compounds, single- or double-stranded RNA interference (RNAi) compounds such as siRNA compounds, modified bases/locked nucleic acids (LNAs), peptide nucleic acids (PNAs), and other oligomeric compounds or oligonucleotide mimetics that hybridize to at least a portion of the target nucleic acid and modulate its function. In some embodiments, the inhibitory nucleic acids include antisense RNA, antisense DNA, chimeric antisense oligonucleotides, antisense oligonucleotides comprising modified linkages, interference RNA (RNAi), short interfering RNA (siRNA); a micro, interfering RNA (miRNA); a small, temporal RNA (stRNA); or a short, hairpin RNA (shRNA); small RNA-induced gene activation (RNAa); small activating RNAs (saRNAs), or combinations thereof. See, e.g., WO 2010040112.

In some embodiments, the inhibitory nucleic acids are 10 to 50, 10 to 20, 10 to 25, 13 to 50, or 13 to 30 nucleotides in length. One having ordinary skill in the art will appreciate that this embodies inhibitory nucleic acids having complementary portions of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length, or any range therewithin. In some embodiments, the inhibitory nucleic acids are 15 nucleotides in length. In some embodiments, the inhibitory nucleic acids are 12 or 13 to 20, 25, or 30 nucleotides in length. One having ordinary skill in the art will appreciate that this embodies inhibitory nucleic acids having complementary portions of 12, 13, 14, 15, 16, 17, 18, 19, 20, 21, 22, 23, 24, 25, 26, 27, 28, 29 or 30 nucleotides in length, or any range therewithin (complementary portions refers to those portions of the inhibitory nucleic acids that are complementary to the target sequence (i.e., the AIM2 sequence)).

The inhibitory nucleic acids useful in the present methods are sufficiently complementary to the target RNA (i.e., AIM2 RNA), i.e., hybridize sufficiently well and with sufficient specificity, to give the desired effect. “Complementary” refers to the capacity for pairing, through hydrogen bonding, between two sequences comprising naturally or non-naturally occurring bases or analogs thereof. For example, if a base at one position of an inhibitory nucleic acid is capable of hydrogen bonding with a base at the corresponding position of a RNA, then the bases are considered to be complementary to each other at that position. 100% complementarity is not required.

Routine methods can be used to design an inhibitory nucleic acid that binds to the target sequence with sufficient specificity. In some embodiments, the methods include using bioinformatics methods known in the art to identify regions of secondary structure, e.g., one, two, or more stem-loop structures, or pseudoknots, and selecting those regions to target with an inhibitory nucleic acid. For example, “gene walk” methods can be used to optimize the inhibitory activity of the nucleic acid; for example, a series of oligonucleotides of 10-30 nucleotides spanning the length of a target RNA can be prepared, followed by testing for activity. Optionally, gaps, e.g., of 5-10 nucleotides or more, can be left between the target sequences to reduce the number of oligonucleotides synthesized and tested. GC content is preferably between about 30-60%. Contiguous runs of three or more Gs or Cs should be avoided where possible (for example, it may not be possible with very short (e.g., about 9-10 nt) oligonucleotides).

In some embodiments, the inhibitory nucleic acid molecules can be designed to target a specific region of the AIM2 RNA sequence. For example, a specific functional region can be targeted, e.g., a region comprising a known RNA localization motif (i.e., a region complementary to the target nucleic acid on which the RNA acts). Alternatively or in addition, highly conserved regions can be targeted, e.g., regions identified by aligning sequences from disparate species such as primate (e.g., human) and rodent (e.g., mouse) and looking for regions with high degrees of identity. For example, highly conserved regions between mouse and human can be targeted, yielding an inhibitory nucleic acid molecule capable of targeting the target molecule in both mouse and human models. Percent identity can be determined routinely using basic local alignment search tools (BLAST programs) (Altschul et al., J. Mol. Biol., 1990, 215, 403-410; Zhang and Madden, Genome Res., 1997, 7, 649-656), e.g., using the default parameters.

Once one or more target regions, segments or sites have been identified, e.g., within a target sequence known in the art or provided herein, inhibitory nucleic acid compounds are chosen that are sufficiently complementary to the target, i.e., that hybridize sufficiently well and with sufficient specificity (i.e., do not substantially bind to other non-target RNAs), to give the desired effect.

In the context of this disclosure, hybridization means hydrogen bonding, which may be Watson-Crick, Hoogsteen or reversed Hoogsteen hydrogen bonding, between complementary nucleoside or nucleotide bases. For example, adenine and thymine are complementary nucleobases which pair through the formation of hydrogen bonds. Complementary, as used herein, refers to the capacity for precise pairing between two nucleotides. For example, if a nucleotide at a certain position of an oligonucleotide is capable of hydrogen bonding with a nucleotide at the same position of a RNA molecule, then the inhibitory nucleic acid and the RNA are considered to be complementary to each other at that position. The inhibitory nucleic acids and the RNA are complementary to each other when a sufficient number of corresponding positions in each molecule are occupied by nucleotides which can hydrogen bond with each other. Thus, “specifically hybridisable” and “complementary” are terms which are used to indicate a sufficient degree of complementarity or precise pairing such that stable and specific binding occurs between the inhibitory nucleic acid and the RNA target. For example, if a base at one position of an inhibitory nucleic acid is capable of hydrogen bonding with a base at the corresponding position of a RNA, then the bases are considered to be complementary to each other at that position. 100% complementarity is not required.

It is understood in the art that a complementary nucleic acid sequence need not be 100% complementary to that of its target nucleic acid to be specifically hybridisable. A complementary nucleic acid sequence for purposes of the present methods is specifically hybridisable when binding of the sequence to the target RNA molecule interferes with the normal function of the target RNA to cause a loss of activity, and there is a sufficient degree of complementarity to avoid non-specific binding of the sequence to non-target RNA sequences under conditions in which specific binding is desired, e.g., under physiological conditions in the case of in vivo assays or therapeutic treatment, and in the case of in vitro assays, under conditions in which the assays are performed under suitable conditions of stringency. For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

In general, the inhibitory nucleic acids useful in the methods described herein have at least 80% sequence complementarity to a target region within the target nucleic acid, e.g., 90%, 95%, or 100% sequence complementarity to the target region within an RNA. For example, an antisense compound in which 18 of 20 nucleobases of the antisense oligonucleotide are complementary, and would therefore specifically hybridize, to a target region would represent 90 percent complementarity. Percent complementarity of an inhibitory nucleic acid with a region of a target nucleic acid can be determined routinely using basic local alignment search tools (BLAST programs) (Altschul et al., J. Mol. Biol., 1990, 215, 403-410; Zhang and Madden, Genome Res., 1997, 7, 649-656). Inhibitory nucleic acids that hybridize to an RNA can be identified through routine experimentation. In general the inhibitory nucleic acids must retain specificity for their target, i.e., must not directly bind to, or directly significantly affect expression levels of, transcripts other than the intended target.

For further disclosure regarding inhibitory nucleic acids, please see US2010/0317718 (antisense oligos); US2010/0249052 (double-stranded ribonucleic acid (dsRNA)); US2009/0181914 and US2010/0234451 (LNAs); US2007/0191294 (siRNA analogues); US2008/0249039 (modified siRNA); and WO2010/129746 and WO2010/040112 (inhibitory nucleic acids).

### Antisense

In some embodiments, the inhibitory nucleic acids are antisense oligonucleotides. Antisense oligonucleotides are typically designed to block expression of a DNA or RNA target by binding to the target and halting expression at the level of transcription, translation, or splicing. Antisense oligonucleotides of the present invention are complementary nucleic acid sequences designed to hybridize under stringent conditions to an RNA. Thus, oligonucleotides are chosen that are sufficiently complementary to the target, i.e., that hybridize sufficiently well and with sufficient specificity, to give the desired effect.

### siRNA/shRNA

In some embodiments, the nucleic acid sequence that is complementary to a target RNA can be an interfering RNA, including but not limited to a small interfering RNA (“siRNA”) or a small hairpin RNA (“shRNA”). Methods for constructing interfering RNAs are well known in the art. For example, the interfering RNA can be assembled from two separate oligonucleotides, where one strand is the sense strand and the other is the antisense strand, wherein the antisense and sense strands are self-complementary (i.e., each strand comprises nucleotide sequence that is complementary to nucleotide sequence in the other strand; such as where the antisense strand and sense strand form a duplex or double stranded structure); the antisense strand comprises nucleotide sequence that is complementary to a nucleotide sequence in a target nucleic acid molecule or a portion thereof (i.e., an undesired gene) and the sense strand comprises nucleotide sequence corresponding to the target nucleic acid sequence or a portion thereof. Alternatively, interfering RNA is assembled from a single oligonucleotide, where the self-complementary sense and antisense regions are linked by means of nucleic acid based or non-nucleic acid-based linker(s). The interfering RNA can be a polynucleotide with a duplex, asymmetric duplex, hairpin or asymmetric hairpin secondary structure, having self-complementary sense and antisense regions, wherein the antisense region comprises a nucleotide sequence that is complementary to nucleotide sequence in a separate target nucleic acid molecule or a portion thereof and the sense region having nucleotide sequence corresponding to the target nucleic acid sequence or a portion thereof. The interfering can be a circular single-stranded polynucleotide having two or more loop structures and a stem comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof, and wherein the circular polynucleotide can be processed either in vivo or in vitro to generate an active siRNA molecule capable of mediating RNA interference.

In some embodiments, the interfering RNA coding region encodes a self-complementary RNA molecule having a sense region, an antisense region and a loop region. Such an RNA molecule when expressed desirably forms a “hairpin” structure, and is referred to herein as an “shRNA.” The loop region is generally between about 2 and about 10 nucleotides in length. In some embodiments, the loop region is from about 6 to about 9 nucleotides in length. In some embodiments, the sense region and the antisense region are between about 15 and about 20 nucleotides in length. Following post-transcriptional processing, the small hairpin RNA is converted into a siRNA by a cleavage event mediated by the enzyme Dicer, which is a member of the RNase III family. The siRNA is then capable of inhibiting the expression of a gene with which it shares homology. For details, see Brummelkamp et al., Science 296:550-553, (2002); Lee et al, Nature Biotechnol., 20, 500-505, (2002); Miyagishi and Taira, Nature Biotechnol 20:497-500, (2002); Paddison et al. Genes & Dev. 16:948-958, (2002); Paul, Nature Biotechnol, 20, 505-508, (2002); Sui, Proc. Natl. Acad. Sd. USA, 99 (6), 5515-5520, (2002); Yu et al. Proc NatlAcadSci USA 99:6047-6052, (2002).

The target RNA cleavage reaction guided by siRNAs is highly sequence specific. In general. siRNA containing a nucleotide sequences identical to a portion of the target nucleic acid are preferred for inhibition. However, 100% sequence identity between the siRNA and the target gene is not required to practice the present invention. Thus the invention has the advantage of being able to tolerate sequence variations that might be expected due to genetic mutation, strain polymorphism, or evolutionary divergence. For example, siRNA sequences with insertions, deletions, and single point mutations relative to the target sequence have also been found to be effective for inhibition. Alternatively, siRNA sequences with nucleotide analog substitutions or insertions can be effective for inhibition. In general the siRNAs must retain specificity for their target, i.e., must not directly bind to, or directly significantly affect expression levels of, transcripts other than the intended target.

Exemplary interfering RNA sequences (sense and antisense strands) of the disclosure are provided in Table 1, below.

TABLE 1
Exemplary Aim2 siRNA constructs and their targets. Legend: m =
2′-O-methyl; f = 2′-fluoro; # = Phosphorothioate bond; P = 5′-Phosphate; TegChol =
3′-Tetraethylene Glycol (Teg) Cholesterol Conjugate; ( ) = Phosphodiester bond

| Target | Base (Unmodified) Sequence | Modified Sequence |
|---|---|---|
| Target position of Aim2 siRNA | Sense: | Aim2 siRNA 2 |
| 2 (SEQ ID NO: 28): | GUUGAAUUAUAUGCA | Sense: |
| Human | (SEQ ID NO: 27) | (mG)#(mU)#(fU)(mG)(fA)(mA) |
| Nucleotides 362-380 for | Antisense: | (fU)(mU)(fA)(mU)(mA)(mU) |
| human AIM2 mRNA (SEQ ID | UGCAUAUAAUUCAACUU | (fG)#(mC)#(mA)-TegChol |
| NO: 46) | CUG (SEQ ID NO: 28) | (SEQ ID NO: 3) |
| Mouse | | Antisense: |
| Nucleotides 662-681 for mouse | | P(mU)#(fG)#(mC)(fA)(fU)(fA) |
| AIM2 mRNA (SEQ ID NO:48) | | (mU)(fA)(mA)(fU)(mU)(fC) |
| | | (mA)#(fA)#(mC)#(fU)#(mU)# |
| | | (mC)#(mU)#(fG) (SEQ ID |
| | | NO: 4) |
| Target position of Aim2 siRNA | Sense: | Aim2 siRNA 4 |
| 4 (SEQ ID NO: 30): | UUUGUAAAAGUUUUA | Sense: |
| Human | (SEQ ID NO: 29) | (mU)#(mU)#(fU)(mG)(fU)(mA) |
| Nucleotides 714-732 for | Antisense: | (fA)(mA)(fA)(mG)(mU)(mU) |
| human AIM2 mRNA (SEQ ID | UAAAACUUUUACAAAGA | (fU)#(mU)#(mA)-TegChol |
| NO: 46) | AGA (SEQ ID NO: 30) | (SEQ ID NO: 7) |
| Mouse | | Antisense: |
| Nucleotides 1034-1051 for | | P(mU)#(fA)#(mA)(fA)(fA)(fC) |
| mouse AIM2 mRNA (SEQ ID | | (mU)(fU)(mU)(fU)(mA)(fC) |
| NO: 48) | | (mA)#(fA)#(mA)#(fG)4(mA)# |
| | | (mA)#(mG)#(fA) (SEQ ID |
| | | NO: 8) |
| Target position of Aim2 siRNA | Sense: | Aim2 siRNA 9 |
| 9 (SEQ ID NO: 32): | GCUGAAAGCUAUAAA | Sense: |
| Mouse | (SEQ ID NO: 31) | (mG)#(mC)#(fU)(mG)(fA)(mA) |
| Nucleotides 941-960 for mouse | Antisense: | (fA)(mG)(fC)(mU)(mA)(mU) |
| Aim2 mRNA (SEQ ID NO: 48) | UUUAUAGCUUUCAGCAC | (fA)#(mA)#(mA)-TegChol |
| | CGU (SEQ ID NO: 32) | (SEQ ID NO: 17) |
| | | Antisense: |
| | | P(mU)#(fU)#(mU)(fA)(fU)(fA) |
| | | (mG)(fC)(mU)(fU)(mU)(fC) |
| | | (mA)#(fG)#(mC)#(fA)#(mC)# |
| | | (mC)#(mG)#(fU) (SEQ ID |
| | | NO: 18) |

In specific embodiments, the interfering RNA is a double stranded RNA molecule comprising a sense strand and an antisense strand, wherein the sense strand comprises the sequence UUUGUAAAAGUUUUA (SEQ ID NO:29), or differs by 1, 2, or 3, nucleotides, and the antisense strand comprises the sequence UAAAACUUUUACAAAGAAGA (SEQ ID NO:30), or differs by 1, 2, or 3 nucleotides. In some embodiments, 1, 2, 3, 4, 5, 6, 7, 8, 9, 10, 11, 12, 13, 14, or all 15 of the nucleotides of the sense strand are modified (e.g., comprise a 2′-fluoro-modified sugar, a 2′-O-methyl-modified sugar, and/or a phosphorothioate backbone modification). In some embodiments, 1, 2, 3, 4, 5, 6, 7, 8, 9, 10, 11, 12, 13, 14, 15, 16, 17, 18, 19, or all 20 of the nucleotides of the antisense strand are modified (e.g., comprise a 2′-fluoro-modified sugar, a 2′-O-methyl-modified sugar, and/or a phosphorothioate backbone modification). In some embodiments, 1, 2, 3, 4, 5, 6, 7, 8, 9, 10, 11, 12, 13, 14, or all 15 of the nucleotides of the sense strand are modified (e.g., comprise a 2′-fluoro-modified sugar, a 2′-O-methyl-modified sugar, and/or a phosphorothioate backbone modification) and 1, 2, 3, 4, 5, 6, 7, 8, 9, 10, 11, 12, 13, 14, 15, 16, 17, 18, 19, or all 20 of the nucleotides of the antisense strand are modified (e.g., comprise a 2′-fluoro-modified sugar, a 2′-O-methyl-modified sugar, and/or a phosphorothioate backbone modification). In some embodiments, the sense strand comprises the sequence (mU)#(mU)#(fU)(mG)(fU)(mA)(fA)(mA)(fA)(mG)(mU)(mU)(fU)#(mU)#(mA)-TegChol (SEQ ID NO:7), wherein m is 2′-O-methyl, f is 2′-fluoro, # is a phosphorothioate bond, P is a 5′-Phosphate, TegChol is 3′-Tetraethylene Glycol (Teg) Cholesterol Conjugate, and ( ) is a phosphodiester bond. In some embodiments, the antisense strand comprises the sequence P(mU)#(fA)#(mA)(fA)(fA)(fC)(mU)(fU)(mU)(fU)(mA)(fC)(mA)#(fA)#(mA)#(fG)#(mA)#(mA)#(mG)#(fA) (SEQ ID NO:8), wherein m is 2′-O-methyl, f is 2′-fluoro, # is a phosphorothioate bond, P is a 5′-Phosphate. TegChol is 3′-Tetraethylene Glycol (Teg) Cholesterol Conjugate, and ( ) is a phosphodiester bond. In some embodiments, the sense strand comprises the sequence (mU)#(mU)#(fU)(mG)(fU)(mA)(fA)(mA)(fA)(mG)(mU)(mU)(fU)#(mU)#(mA)-TegChol (SEQ ID NO:7) and the antisense strand comprises the sequence P(mU)#(fA)#(mA)(fA)(fA)(fC)(mU)(fU)(mU)(fU)(mA)(fC)(mA)#(fA)#(mA)#(fG)#(mA)#(mA)#(mG)#(fA) (SEQ ID NO:8), m is 2′-O-methyl, f is 2′-fluoro, # is a phosphorothioate bond, P is a 5′-Phosphate, TegChol is 3′-Tetraethylene Glycol (Teg) Cholesterol Conjugate, and ( ) is a phosphodiester bond. In some embodiments, TegChol is replaced with docosahexaenoic acid (DHA). In some embodiments, the 5′-phosphate of the antisense strand is replaced with a 5′-(E)-vinylphosphonate.

In specific embodiments, the interfering RNA is a double stranded RNA molecule comprising a sense strand and an antisense strand, wherein the sense strand comprises the sequence GUUGAAUUAUAUGCA (SEQ ID NO:27), or differs by 1, 2, or 3, nucleotides, and the antisense strand comprises the sequence UGCAUAUAAUUCAACUUCUG (SEQ ID NO:28), or differs by 1, 2, or 3 nucleotides. In some embodiments, 1, 2, 3, 4, 5, 6, 7, 8, 9, 10, 11, 12, 13, 14, or all 15 of the nucleotides of the sense strand are modified (e.g., comprise a 2′-fluoro-modified sugar, a 2′-O-methyl-modified sugar, and/or a phosphorothioate backbone modification). In some embodiments, 1, 2, 3, 4, 5, 6, 7, 8, 9, 10, 11, 12, 13, 14, 15, 16, 17, 18, 19, or all 20 of the nucleotides of the antisense strand are modified (e.g., comprise a 2′-fluoro-modified sugar, a 2′-O-methyl-modified sugar, and/or a phosphorothioate backbone modification). In some embodiments, 1, 2, 3, 4, 5, 6, 7, 8, 9, 10, 11, 12, 13, 14, or all 15 of the nucleotides of the sense strand are modified (e.g., comprise a 2′-fluoro-modified sugar, a 2′-O-methyl-modified sugar, and/or a phosphorothioate backbone modification) and 1, 2, 3, 4, 5, 6, 7, 8, 9, 10, 11, 12, 13, 14, 15, 16, 17, 18, 19, or all 20 of the nucleotides of the antisense strand are modified (e.g., comprise a 2′-fluoro-modified sugar, a 2′-O-methyl-modified sugar, and/or a phosphorothioate backbone modification). In some embodiments, the sense strand comprises the sequence (mG)#(mU)#(fU)(mG)(fA)(mA)(fU)(mU)(fA)(mU)(mA)(mU)(fG)#(mC)#(mA)-TegChol (SEQ ID NO:3), wherein m is 2′-O-methyl, f is 2′-fluoro, # is a phosphorothioate bond, P is a 5′-Phosphate, TegChol is 3′-Tetraethylene Glycol (Teg) Cholesterol Conjugate, and ( ) is a phosphodiester bond. In some embodiments, the antisense strand comprises the sequence P(mU)#(fG)#(mC)(fA)(fU)(fA)(mU)(fA)(mA)(fU)(mU)(fC)(mA)#(fA)#(mC)#(fU)#(mU)#(mC)#(mU)#(fG) (SEQ ID NO:4), wherein m is 2′-O-methyl, f is 2′-fluoro, # is a phosphorothioate bond, P is a 5′-Phosphate, TegChol is 3′-Tetraethylene Glycol (Teg) Cholesterol Conjugate, and ( ) is a phosphodiester bond. In some embodiments, the sense strand comprises the sequence (mG)#(mU)#(fU)(mG)(fA)(mA)(fU)(mU)(fA)(mU)(mA)(mU)(fG)#(mC)#(mA)-TegChol (SEQ ID NO:3) and the antisense strand comprises the sequence P(mU)#(fG)#(mC)(fA)(fU)(fA)(mU)(fA)(mA)(fU)(mU)(fC)(mA)#(fA)#(mC)#(fU)#(mU)#(mC)#(mU)#(fG) (SEQ ID NO:4), m is 2′-O-methyl, f is 2′-fluoro, # is a phosphorothioate bond, P is a 5′-Phosphate, TegChol is 3′-Tetraethylene Glycol (Teg) Cholesterol Conjugate, and ( ) is a phosphodiester bond. In some embodiments, TegChol is replaced with docosahexaenoic acid (DHA). In some embodiments, the 5′-phosphate of the antisense strand is replaced with a 5′-(E)-vinylphosphonate.

In specific embodiments, the interfering RNA is a double stranded RNA molecule comprising a sense strand and an antisense strand, wherein the sense strand comprises the sequence GCUGAAAGCUAUAAA (SEQ ID NO:31), or differs by 1, 2, or 3, nucleotides, and the antisense strand comprises the sequence UUUAUAGCUUUCAGCACCGU (SEQ ID NO:32), or differs by 1, 2, or 3 nucleotides. In some embodiments, 1, 2, 3, 4, 5, 6, 7, 8, 9, 10, 11, 12, 13, 14, or all 15 of the nucleotides of the sense strand are modified (e.g., comprise a 2′-fluoro-modified sugar, a 2′-O-methyl-modified sugar, and/or a phosphorothioate backbone modification). In some embodiments, 1, 2, 3, 4, 5, 6, 7, 8, 9, 10, 11, 12, 13, 14, 15, 16, 17, 18, 19, or all 20 of the nucleotides of the antisense strand are modified (e.g., comprise a 2′-fluoro-modified sugar, a 2′-O-methyl-modified sugar, and/or a phosphorothioate backbone modification). In some embodiments, 1, 2, 3, 4, 5, 6, 7, 8, 9, 10, 11, 12, 13, 14, or all 15 of the nucleotides of the sense strand are modified (e.g., comprise a 2′-fluoro-modified sugar, a 2′-O-methyl-modified sugar, and/or a phosphorothioate backbone modification) and 1, 2, 3, 4, 5, 6, 7, 8, 9, 10, 11, 12, 13, 14, 15, 16, 17, 18, 19, or all 20 of the nucleotides of the antisense strand are modified (e.g., comprise a 2′-fluoro-modified sugar, a 2′-O-methyl-modified sugar, and/or a phosphorothioate backbone modification). In some embodiments, the sense strand comprises the sequence (mG)#(mC)#(fU)(mG)(fA)(mA)(fA)(mG)(fC)(mU)(mA)(mU)(fA)#(mA)#(mA)-TegChol (SEQ ID NO:17), wherein m is 2′-O-methyl, f is 2′-fluoro, # is a phosphorothioate bond, P is a 5′-Phosphate, TegChol is 3′-Tetraethylene Glycol (Teg) Cholesterol Conjugate, and ( ) is a phosphodiester bond. In some embodiments, the antisense strand comprises the sequence P(mU)#(fU)#(mU)(fA)(fU)(fA)(mG)(fC)(mU)(fU)(mU)(fC)(mA)#(fG)#(mC)#(fA)#(mC)#(mC)#(mG)#(fU) (SEQ ID NO:18), wherein m is 2′-O-methyl, f is 2′-fluoro, # is a phosphorothioate bond, P is a 5′-Phosphate, TegChol is 3′-Tetraethylene Glycol (Teg) Cholesterol Conjugate, and ( ) is a phosphodiester bond. In some embodiments, the sense strand comprises the sequence (mG)#(mC)#(fU)(mG)(fA)(mA)(fA)(mG)(fC)(mU)(mA)(mU)(fA)#(mA)#(mA)-TegChol (SEQ ID NO:17) and the antisense strand comprises the sequence P(mU)#(fU)#(mU)(fA)(fU)(fA)(mG)(fC)(mU)(fU)(mU)(fC)(mA)#(fG)#(mC)#(fA)#(mC)#(mC)#(mG)#(fU) (SEQ ID NO:18), m is 2′-O-methyl, f is 2′-fluoro, # is a phosphorothioate bond, P is a 5′-Phosphate, TegChol is 3′-Tetraethylene Glycol (Teg) Cholesterol Conjugate, and ( ) is a phosphodiester bond. In some embodiments, TegChol is replaced with docosahexaenoic acid (DHA). In some embodiments, the 5′-phosphate of the antisense strand is replaced with a 5′-(E)-vinylphosphonate.

### Ribozymes

Trans-cleaving enzymatic nucleic acid molecules can also be used; they have shown promise as therapeutic agents for human disease (Usman & McSwiggen, 1995 Ann. Rep. Med. Chem. 30, 285-294; Christoffersen and Man, 1995 J. Med. Chem. 38, 2023-2037). Enzymatic nucleic acid molecules can be designed to cleave specific RNA targets within the background of cellular RNA. Such a cleavage event renders the RNA non-functional.

In general, enzymatic nucleic acids with RNA cleaving activity act by first binding to a target RNA. Such binding occurs through the target binding portion of a enzymatic nucleic acid which is held in close proximity to an enzymatic portion of the molecule that acts to cleave the target RNA. Thus, the enzymatic nucleic acid first recognizes and then binds a target RNA through complementary base pairing, and once bound to the correct site, acts enzymatically to cut the target RNA. Strategic cleavage of such a target RNA will destroy its ability to direct synthesis of an encoded protein. After an enzymatic nucleic acid has bound and cleaved its RNA target, it is released from that RNA to search for another target and can repeatedly bind and cleave new targets.

Several approaches such as in vitro selection (evolution) strategies (Orgel, 1979, Proc. R. Soc. London, B 205, 435) have been used to evolve new nucleic acid catalysts capable of catalyzing a variety of reactions, such as cleavage and ligation of phosphodiester linkages and amide linkages, (Joyce, 1989, Gene, 82, 83-87; Beaudry et al., 1992, Science 257, 635-641; Joyce, 1992, Scientific American 267, 90-97; Breaker et al, 1994, TIBTECH 12, 268; Bartel et al, 1993, Science 261 :1411-1418; Szostak, 1993, TIBS 17, 89-93; Kumar et al, 1995, FASEB J., 9, 1183; Breaker, 1996, Curr. Op. Biotech., 1, 442). The development of ribozymes that are optimal for catalytic activity would contribute significantly to any strategy that employs RNA-cleaving ribozymes for the purpose of regulating gene expression. The hammerhead ribozyme, for example, functions with a catalytic rate (kcat) of about 1 min⁻¹ in the presence of saturating (10 rnM) concentrations of Mg²⁺ cofactor. An artificial “RNA ligase” ribozyme has been shown to catalyze the corresponding self-modification reaction with a rate of about 100 min⁻¹. In addition, it is known that certain modified hammerhead ribozymes that have substrate binding arms made of DNA catalyze RNA cleavage with multiple turn-over rates that approach 100 min⁻¹.

### Modified Inhibitory Nucleic Acids

In some embodiments, the inhibitory nucleic acids described herein are modified, e.g., comprise one or more modified bonds or bases. A number of modified bases include phosphorothioate, methylphosphonate, peptide nucleic acids, or locked nucleic acid (LNA) molecules. Some inhibitory nucleic acids are fully modified, while others are chimeric and contain two or more chemically distinct regions, each made up of at least one nucleotide. These inhibitory nucleic acids typically contain at least one region of modified nucleotides that confers one or more beneficial properties (such as, for example, increased nuclease resistance, increased uptake into cells, increased binding affinity for the target) and a region that is a substrate for enzymes capable of cleaving RNA:DNA or RNA:RNA hybrids. Chimeric inhibitory nucleic acids of the invention may be formed as composite structures of two or more oligonucleotides, modified oligonucleotides, oligonucleosides and/or oligonucleotide mimetics as described above. Such compounds have also been referred to in the art as hybrids or gapmers. In some embodiments, the oligonucleotide is a gapmer (contain a central stretch (gap) of DNA monomers sufficiently long to induce RNase H cleavage, flanked by blocks of LNA modified nucleotides; see, e.g., Stanton et al., Nucleic Acid Ther. 2012. 22: 344-359; Nowotny et al., Cell, 121:1005-1016, 2005; Kurreck, European Journal of Biochemistry 270:1628-1644, 2003; FLuiter et al., Mol Biosyst. 5 (8):838-43, 2009). In some embodiments, the oligonucleotide is a mixmer (includes alternating short stretches of LNA and DNA; Naguibneva et al., Biomed Pharmacother. 2006 November; 60 (9):633-8; Ørom et al., Gene. 2006 May 10; 372 ( ):137-41). Representative United States patents that teach the preparation of such hybrid structures comprise, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, each of which is herein incorporated by reference.

In some embodiments, the inhibitory nucleic acid comprises at least one nucleotide modified at the 2′ position of the sugar, most preferably a 2′-O-alkyl, 2′-O-alkyl-O-alkyl or 2′-fluoro-modified nucleotide. In other preferred embodiments, RNA modifications include 2′-fluoro, 2′-amino and 2′ O-methyl modifications on the ribose of pyrimidines, abasic residues or an inverted base at the 3′ end of the RNA. Such modifications are routinely incorporated into oligonucleotides and these oligonucleotides have been shown to have a higher Tm (i.e., higher target binding affinity) than; 2′-deoxyoligonucleotides against a given target.

A number of nucleotide and nucleoside modifications have been shown to make the oligonucleotide into which they are incorporated more resistant to nuclease digestion than the native oligodeoxynucleotide; these modified oligos survive intact for a longer time than unmodified oligonucleotides. Specific examples of modified oligonucleotides include those comprising modified backbones, for example, phosphorothioates, phosphotriesters, methyl phosphonates, short chain alkyl or cycloalkyl intersugar linkages or short chain heteroatomic or heterocyclic intersugar linkages. Most preferred are oligonucleotides with phosphorothioate backbones and those with heteroatom backbones, particularly CH₂—NH—O—CH₂, CH, —N(CH₃)—O—CH₂ (known as a methylene(methylimino) or MMI backbone], CH₂—O—N(CH₃)—CH₂, CH₂—N(CH₃)—N(CH₃)—CH₂ and O—N(CH₃)—CH₂—CH₂ backbones, wherein the native phosphodiester backbone is represented as O—P—O—CH,); amide backbones (see De Mesmaeker et al. Ace. Chem. Res. 1995, 28:366-374); morpholino backbone structures (see Summerton and Weller, U.S. Pat. No. 5,034,506); peptide nucleic acid (PNA) backbone (wherein the phosphodiester backbone of the oligonucleotide is replaced with a polyamide backbone, the nucleotides being bound directly or indirectly to the aza nitrogen atoms of the polyamide backbone, see Nielsen et al., Science 1991, 254, 1497). Phosphorus-containing linkages include, but are not limited to, phosphorothioates, chiral phosphorothioates, phosphorodithioates, phosphotriesters, aminoalkylphosphotriesters, methyl and other alkyl phosphonates comprising 3′alkylene phosphonates and chiral phosphonates, phosphinates, phosphoramidates comprising 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2; see U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050.

Morpholino-based oligomeric compounds are described in Dwaine A. Braasch and David R. Corey, Biochemistry, 2002, 41 (14), 4503-4510); Genesis, volume 30, issue 3, 2001; Heasman, J., Dev. Biol., 2002, 243, 209-214; Nasevicius et al., Nat. Genet., 2000, 26, 216-220; Lacerra et al., Proc. Natl. Acad. Sci., 2000, 97, 9591-9596; and U.S. Pat. No. 5,034,506, issued Jul. 23, 1991.

Cyclohexenyl nucleic acid oligonucleotide mimetics are described in Wang et al., J. Am. Chem. Soc., 2000, 122, 8595-8602.

Modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These comprise those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts; see U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference.

One or more substituted sugar moieties can also be included, e.g., one of the following at the 2′ position: OH, SH, SCH₃, F, OCN, OCH₃OCH₃, OCH₃O(CH₂)ₙCH₃, O(CH₂)ₙNH₂ or O(CH₂)ₙCH₃ where n is from 1 to about 10; Ci to C10 lower alkyl, alkoxyalkoxy, substituted lower alkyl, alkaryl or aralkyl; Cl; Br; CN; CF₃; OCF₃; O-, S-, or N-alkyl; O-, S-, or N-alkenyl; SOCH₃; SO₂ CH₃; ONO₂; NO₂; N₃; NH₂; heterocycloalkyl; heterocycloalkaryl; aminoalkylamino; polyalkylamino; substituted silyl; an RNA cleaving group; a reporter group; an intercalator; a group for improving the pharmacokinetic properties of an oligonucleotide; or a group for improving the pharmacodynamic properties of an oligonucleotide and other substituents having similar properties. A preferred modification includes 2′-methoxyethoxy [2′-0-CH₂CH₂OCH₃, also known as 2′-O-(2-methoxyethyl)] (Martin et al, HeIv. Chim. Acta, 1995, 78, 486). Other preferred modifications include 2′-methoxy (2′-0-CH₃), 2′-propoxy (2′-OCH₂ CH₂CH₃) and 2′-fluoro (2′-F). Similar modifications may also be made at other positions on the oligonucleotide, particularly the 3′ position of the sugar on the 3′ terminal nucleotide and the 5′ position of 5′ terminal nucleotide. Oligonucleotides may also have sugar mimetics such as cyclobutyls in place of the pentofuranosyl group.

Inhibitory nucleic acids can also include, additionally or alternatively, nucleobase (often referred to in the art simply as “base”) modifications or substitutions. As used herein, “unmodified” or “natural” nucleobases include adenine (A), guanine (G), thymine (T), cytosine (C) and uracil (U). Modified nucleobases include nucleobases found only infrequently or transiently in natural nucleic acids, e.g., hypoxanthine, 6-methyladenine, 5-Me pyrimidines, particularly 5-methylcytosine (also referred to as 5-methyl-2′ deoxycytosine and often referred to in the art as 5-Me-C), 5-hydroxymethylcytosine (HMC), glycosyl HMC and gentobiosyl HMC, as well as synthetic nucleobases, e.g., 2-aminoadenine, 2-(methylamino)adenine, 2-(imidazolylalkyl)adenine, 2-(aminoalklyamino)adenine or other heterosubstituted alkyladenines, 2-thiouracil, 2-thiothymine, 5-bromouracil, 5-hydroxymethyluracil, 8-azaguanine, 7-deazaguanine, N6 (6-aminohexyl)adenine and 2,6-diaminopurine. Kornberg, A., DNA Replication, W. H. Freeman & Co., San Francisco, 1980, pp75-77; Gebeyehu, G., et al. Nucl. Acids Res. 1987, 15:4513). A “universal” base known in the art, e.g., inosine, can also be included. 5-Me-C substitutions have been shown to increase nucleic acid duplex stability by 0.6-1.2<0>C. (Sanghvi, Y. S., in Crooke, S. T. and Lebleu, B., eds., Antisense Research and Applications, CRC Press, Boca Raton, 1993, pp. 276-278) and are presently preferred base substitutions.

It is not necessary for all positions in a given oligonucleotide to be uniformly modified, and in fact more than one of the aforementioned modifications may be incorporated in a single oligonucleotide or even at within a single nucleoside within an oligonucleotide.

In some embodiments, both a sugar and an internucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, for example, an aminoethylglycine backbone. The nucleobases are retained and are bound directly or indirectly to aza nitrogen atoms of the amide portion of the backbone. Representative United States patents that teach the preparation of PNA compounds comprise, but are not limited to, U.S. Pat. Nos. 5,539,082; 5,714,331; and 5,719,262, each of which is herein incorporated by reference. Further teaching of PNA compounds can be found in Nielsen et al, Science, 1991, 254, 1497-1500.

Inhibitory nucleic acids can also include one or more nucleobase (often referred to in the art simply as “base”) modifications or substitutions. As used herein, “unmodified” or “natural” nucleobases comprise the purine bases adenine (A) and guanine (G), and the pyrimidine bases thymine (T), cytosine (C) and uracil (U). Modified nucleobases comprise other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudo-uracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylquanine and 7-methyladenine, 8-azaguanine and 8-azaadenine, 7-deazaguanine and 7-deazaadenine and 3-deazaguanine and 3-deazaadenine.

Further, nucleobases comprise those disclosed in U.S. Pat. No. 3,687,808, those disclosed in ‘The Concise Encyclopedia of Polymer Science And Engineering’, pages 858-859, Kroschwitz, J. I., ed. John Wiley & Sons, 1990, those disclosed by Englisch et al., Angewandle Chemie, International Edition’, 1991, 30, page 613, and those disclosed by Sanghvi, Y. S., Chapter 15, Antisense Research and Applications’, pages 289-302, Crooke, S. T. and Lebleu, B. ea., CRC Press, 1993. Certain of these nucleobases are particularly useful for increasing the binding affinity of the oligomeric compounds of the invention. These include 5-substituted pyrimidines, 6-azapyrimidines and N-2, N-6 and 0-6 substituted purines, comprising 2-aminopropyladenine, 5-propynyluracil and 5-propynylcytosine. 5-methylcytosine substitutions have been shown to increase nucleic acid duplex stability by 0.6-1.2<0>C (Sanghvi, Y. S., Crooke, S. T. and Lebleu, B., eds, ‘Antisense Research and Applications’, CRC Press, Boca Raton, 1993, pp. 276-278) and are presently preferred base substitutions, even more particularly when combined with 2′-O-methoxyethyl sugar modifications. Modified nucleobases are described in U.S. Pat. Nos. 3,687,808, as well as 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,596,091; 5,614,617; 5,750,692, and 5,681,941, each of which is herein incorporated by reference.

In some embodiments, the inhibitory nucleic acids are chemically linked to one or more moieties or conjugates that enhance the activity, cellular distribution, or cellular uptake of the oligonucleotide. Such moieties comprise but are not limited to, lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al, Ann. N. Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Mancharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-t oxycholesterol moiety (Crooke et al., J. Pharmacol. Exp. Ther., 1996, 277, 923-937). See also U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292.873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, each of which is herein incorporated by reference.

These moieties or conjugates can include conjugate groups covalently bound to functional groups such as primary or secondary hydroxyl groups. Conjugate groups of the invention include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugate groups include cholesterols, lipids, phospholipids, biotin, phenazine, folate, phenanthridine, anthraquinone, acridine, fluoresceins, rhodamines, coumarins, and dyes. Groups that enhance the pharmacodynamic properties, in the context of this invention, include groups that improve uptake, enhance resistance to degradation, and/or strengthen sequence-specific hybridization with the target nucleic acid. Groups that enhance the pharmacokinetic properties, in the context of this invention, include groups that improve uptake, distribution, metabolism or excretion of the compounds of the present invention. Representative conjugate groups are disclosed in International Patent Application No. PCT/US92/09196, filed Oct. 23, 1992, and U.S. Pat. No. 6,287,860, which are incorporated herein by reference. Conjugate moieties include, but are not limited to, lipid moieties such as a cholesterol moiety, cholic acid, a thioether, e.g., hexyl-5-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxy cholesterol moiety. See, e.g., U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941.

### Locked Nucleic Acids (LNAs)

In some embodiments, the modified inhibitory nucleic acids described herein comprise locked nucleic acid (LNA) molecules, e.g., including [alpha]-L-LNAs. LNAs comprise ribonucleic acid analogues wherein the ribose ring is “locked” by a methylene bridge between the 2′-oxygen and the 4′-carbon—i.e., oligonucleotides containing at least one LNA monomer, that is, one 2′-O,4′-C-methylene-β-D-ribofuranosyl nucleotide. LNA bases form standard Watson-Crick base pairs but the locked configuration increases the rate and stability of the basepairing reaction (Jepsen et al., Oligonucleotides, 14, 130-146 (2004)). LNAs also have increased affinity to base pair with RNA as compared to DNA. These properties render LNAs especially useful as probes for fluorescence in situ hybridization (FISH) and comparative genomic hybridization, as knockdown tools for miRNAs, and as antisense oligonucleotides to target mRNAs or other RNAs, e.g., RNAs as described herein.

The LNA molecules can include molecules comprising 10-30, e.g., 12-24, e.g., 12, 13, 14, 15, 16, 17, 18, 19, 20, 21, 22, 23, 24, 25, 26, 27, 28, 29, or 30 nucleotides in each strand, wherein one of the strands is substantially identical, e.g., at least 80% (or more, e.g., 85%, 90%, 95%, or 100%) identical, e.g., having 3, 2, 1, or 0 mismatched nucleotide(s), to a target region in the RNA. The LNA molecules can be chemically synthesized using methods known in the art.

The LNA molecules can be designed using any method known in the art; a number of algorithms are known, and are commercially available (e.g., on the internet, for example at exiqon.com). See, e.g., You et al., Nuc. Acids. Res. 34:e60 (2006); McTigue et al., Biochemistry 43:5388-405 (2004); and Levin et al., Nuc. Acids. Res. 34:e142 (2006). For example, “gene walk” methods, similar to those used to design antisense oligos, can be used to optimize the inhibitory activity of the LNA; for example, a series of oligonucleotides of 10-30 nucleotides spanning the length of a target RNA can be prepared, followed by testing for activity. Optionally, gaps, e.g., of 5-10 nucleotides or more, can be left between the LNAs to reduce the number of oligonucleotides synthesized and tested. GC content is preferably between about 30-60%. General guidelines for designing LNAs are known in the art; for example, LNA sequences will bind very tightly to other LNA sequences, so it is preferable to avoid significant complementarity within an LNA. Contiguous runs of more than four LNA residues, should be avoided where possible (for example, it may not be possible with very short (e.g., about 9-10 nt) oligonucleotides). In some embodiments, the LNAs are xylo-LNAs.

For additional information regarding LNAs see U.S. Pat. Nos. 6,268,490; 6,734,291; 6,770,748; 6,794,499; 7,034,133; 7,053,207; 7,060,809; 7,084,125; and 7,572,582; and U.S. Pre-Grant Pub. Nos. 20100267018; 20100261175; and 20100035968; Koshkin et al. Tetrahedron 54, 3607-3630 (1998); Obika et al. Tetrahedron Lett. 39, 5401-5404 (1998); Jepsen et al., Oligonucleotides 14:130-146 (2004); Kauppinen et al., Drug Disc. Today 2 (3):287-290 (2005); and Ponting et al., Cell 136 (4):629-641 (2009), and references cited therein.

### Making and Using Inhibitory Nucleic Acids

The nucleic acid sequences used to practice the methods described herein, whether RNA, cDNA, genomic DNA, vectors, viruses or hybrids thereof, can be isolated from a variety of sources, genetically engineered, amplified, and/or expressed/generated recombinantly. Recombinant nucleic acid sequences can be individually isolated or cloned and tested for a desired activity. Any recombinant expression system can be used, including, e.g., in vitro, bacterial, fungal, mammalian, yeast, insect or plant cell expression systems.

Nucleic acid sequences of the invention can be inserted into vectors and expressed from transcription units within the vectors. The recombinant vectors can be DNA plasmids or viral vectors. Generation of the vector construct can be accomplished using any suitable genetic engineering techniques well known in the art, including, without limitation, the standard techniques of PCR, oligonucleotide synthesis, restriction endonuclease digestion, ligation, transformation, plasmid purification, and DNA sequencing, for example as described in Sambrook et al. Molecular Cloning: A Laboratory Manual. (1989)), Coffin et al. (Retroviruses. (1997)) and “RNA Viruses: A Practical Approach” (Alan J. Cann, Ed., Oxford University Press, (2000)). As will be apparent to one of ordinary skill in the art, a variety of suitable vectors are available for transferring nucleic acids of the invention into cells. In addition to DNA plasmids or viral vectors, lipid-based vectors may also be used for delivery of nucleic acids described herein into a cell. The selection of an appropriate vector to deliver nucleic acids and optimization of the conditions for insertion of the selected expression vector into the cell, are within the scope of one of ordinary skill in the art without the need for undue experimentation. Viral vectors comprise a nucleotide sequence having sequences for the production of recombinant virus in a packaging cell. Viral vectors expressing nucleic acids of the invention can be constructed based on viral backbones including, but not limited to, a retrovirus, lentivirus, adenovirus, adeno-associated virus, pox virus or alphavirus. The recombinant vectors capable of expressing the nucleic acids of the invention can be delivered as described herein, and persist in target cells (e.g., stable transformants).

Nucleic acid sequences used to practice this invention can be synthesized in vitro by well-known chemical synthesis techniques, as described in, e.g., Adams (1983) J. Am. Chem. Soc. 105:661; Belousov (1997) Nucleic Acids Res. 25:3440-3444; Frenkel (1995) Free Radic. Biol. Med. 19:373-380; Blommers (1994) Biochemistry 33:7886-7896; Narang (1979) Meth. Enzymol. 68:90; Brown (1979) Meth. Enzymol. 68:109; Beaucage (1981) Tetra. Left. 22:1859; U.S. Pat. No. 4,458,066.

Nucleic acid sequences of the invention can be stabilized against nucleolytic degradation such as by the incorporation of a modification, e.g., a nucleotide modification. For example, nucleic acid sequences of the invention includes a phosphorothioate at least the first, second, or third internucleotide linkage at the 5′ or 3′ end of the nucleotide sequence. As another example, the nucleic acid sequence can include a 2′-modified nucleotide, e.g., a 2′-deoxy, 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA). As another example, the nucleic acid sequence can include at least one 2′-O-methyl-modified nucleotide, and in some embodiments, all of the nucleotides include a 2′-O-methyl modification. In some embodiments, the nucleic acids are “locked,” i.e., comprise nucleic acid analogues in which the ribose ring is “locked” by a methylene bridge connecting the 2′-O atom and the 4′-C atom (see, e.g., Kaupinnen et al., Drug Disc. Today 2 (3):287-290 (2005); Koshkin et al., J. Am. Chem. Soc., 120 (50):13252-13253 (1998)). For additional modifications see US 20100004320, US 20090298916, and US 20090143326.

Techniques for the manipulation of nucleic acids used to practice this invention, such as, e.g., subcloning, labeling probes (e.g., random-primer labeling using Klenow polymerase, nick translation, amplification), sequencing, hybridization and the like are well described in the scientific and patent literature, see, e.g., Sambrook et al., Molecular Cloning; A Laboratory Manual 3d ed. (2001); Current Protocols in Molecular Biology, Ausubel et al., eds. (John Wiley & Sons, Inc., New York 2010); Kriegler, Gene Transfer and Expression: A Laboratory Manual (1990); Laboratory Techniques In Biochemistry And Molecular Biology: Hybridization With Nucleic Acid Probes, Part I Theory and Nucleic Acid Preparation, Tijssen, ed. Elsevier, N.Y. (1993).

Techniques for producing self-delivering RNAs are known in the art (see, e.g., Khvorova, A., and Watts, J. K. (2017). Nat. Biotechnol. 35, 238-248; Byrne et al., (2013) J. Ocul. Pharmacol. Ther. 29, 855-864). For example, the sequence of the siRNA targeting human and/or mouse Aim2 gene may be selected to comply with standard siRNA design parameters (see, e.g., Birmingham A., et al., (2007) Nat Protoc 2, 2068-78), including assessment of GC content, specificity and low seed compliment frequency (see, e.g., Anderson E., et al., (2008) Methods Mol Biol 442, 45-63), elimination of sequences containing miRNA seeds, and examination of thermodynamic bias. The resulting oligonucleotides may be synthesized using standard and modified (2-fluoro, 2-O-methyl) phosphoroamidite under solid-phase synthesis conditions on, e.g., a 0.2-1 μmole using a MerMade 12 (BioAutomation) and Expedite ABI DNA/RNA synthesizer (ABI 8909). The oligonucleotides may then be removed from controlled pore glass (CPG), deprotected, and high-performance liquid chromatography (HPLC) purified as described in scientific literature (see, e.g., Alterman J F., et al., (2015) Mol Ther Nucleic Acids 4, e266; Hassler M R., et al., (2018) Nucleic Acids Res.). Purified oligonucleotides may be lyophilized to dryness, reconstituted in water, and passed over a Hi-Trap cation exchange column to exchange the tetraethylammonium counter-ion with sodium. The identity of oligonucleotides may be established by liquid chromatographymass spectrometry (LC-MS) analysis (Waters Q-TOF premier). The relative degree of hydrophobicity of sense strands may be assayed by reverse-phase HPLC (Waters Symmetric 3.5 μm, 4.6×75 mm column) using, e.g., a 0-100% gradient over 15 minutes at 60° C. with 0.1% TEAA in water (eluent A) and 100% acetonitrile (eluent B). Peaks may be monitored at 260 nm.

### Dendritic Cell Vaccines

An ex vivo strategy for treating an AIM2-expressing disease in a subject can involve contacting dendritic cells obtained from the subject with an AIM2 inhibitor described herein (e.g., an AIM2 inhibitory nucleic acid). Alternatively, the dendritic cells can be transfected with a nucleic acid (e.g., a vector) encoding one or more of the AIM2 inhibitors described herein (e.g., an AIM2 inhibitory nucleic acid). After contacting the dendritic cells with the AIM2 inhibitor (e.g., an AIM2 inhibitory nucleic acid) or nucleic acid (e.g., vector), the cells can, optionally, be cultured for a period of time and under conditions that (1) permit expression of the AIM2 inhibitor and (2) permit AIM2 to be inhibited (e.g., until AIM2 expression is reduced by at least 50%, at least 60%, at least 70%, at least 80%, at least 90%, or at least 95%, as determined by, e.g., western blot or PCR). The transfection method will depend on the type of cell and nucleic acid being transfected into the cell. Following the contacting or transfection, the cells are then returned to the subject. For example, in some embodiments, the dendritic cells can be contacted with an AIM2 inhibitor (e.g., an AIM2 inhibitory nucleic acid) or nucleic acid and cultured for, e.g., 7, 8, 9, 10, 11, 12, 13, 14, 15, 16, 17, 18, 19, 20, 21, or 22 days before administering the contacted dendritic cells to the subject. Thus, the disclosure also provides a dendritic cell having reduced AIM2 expression (e.g., wherein expression in the dendritic cell is reduced by at least 50%, at least 60%, at least 70%, at least 80%, at least 90%, or at least 95%, as determined by, e.g., western blot or PCR).

In some embodiments of any of the ex vivo methods, cells that are obtained from the subject, or from a subject of the same species other than the subject (allogeneic) can be contacted with the reagents (or immunogenic/antigenic compositions) and administered to the subject.

In some embodiments, the composition comprises at least 10⁴, 10⁵, 10⁶, 10⁷, 10⁸, or 10⁹ dendritic cells. In some embodiments, the composition comprises less than 10⁵, 10⁶, 10⁷, 10⁸, 10⁹, or 10¹⁰ dendritic cells.

### Preparation of Dendritic Cells

Dendritic cells suitable for administration to subjects (e.g., melanoma patients) can be isolated or obtained from any tissue in which such cells are found, or can be otherwise cultured and provided. Dendritic cells can be found, by way of example, in the bone marrow or peripheral blood mononuclear cells (PBMC) of a mammal or in the spleen of a mammal. For instance, bone marrow can be harvested from a mammal and cultured in a medium that promotes the growth of dendritic cells. GM-CSF, IL-4 and/or other cytokines (e.g., TNF-α), growth factors and supplements can be included in this medium. After a suitable amount of time in culture in medium containing appropriate cytokines (e.g., suitable to expand and differentiate the dendritic cells into mature dendritic cells, e.g., 4, 6, 8, 10, 12, or 14 days), clusters of dendritic cell cultured in the presence of antigens of interest (e.g., in the presence of one or more peptide epitopes of PMEL when treating melanoma) and harvested for use in a cancer vaccine using standard techniques. Antigens (e.g., isolated or purified peptides, or synthetic peptides) can be added to cultures at a concentration of 1 μg/ml-50 μg/ml per antigen, e.g., 2, 5, 10, 20, 30, or 40 μg/ml per antigen.

Methods of producing dendritic cells are known in the art (see, e.g., Freudenthal and Steinman, Proc. Nat. Acad. Sci. USA, 57: 7698-7702, 1990; Helft et al., Immunity, 42: 1197-1211, 2015; Lou et al., Cancer Res., 64: 6783-6790, 2004; Lutz et al., J. Immunol. Methods, 223: 77-92, 1999; Macatonia et al., Immunol., 67: 285-289, 1989; Markowicz and Engleman, J. Clin. Invest., 85: 955-961, 1990; Mehta-Damani et al., J. Immunol., 153: 996-1003, 1994; O'Doherty et al., J. Exp. Med., 178: 1067-1078, 1993; Thomas et al., J. Immunol., 151: 6840-6852, 1993; Young and Steinman, J. Exp. Med., 171: 1315-1332, 1990). One method for isolating DCs from human peripheral blood is described in U.S. Pat. No. 5,643,786, which is incorporated by reference herein in its entirety.

For example, in some embodiments, bone marrow-derived dendritic cells (BMDCs) are generated by harvesting bone marrow cells from a subject, filtering said cells through a 70 μm nylon strainer, lysing the red blood cells with lysis buffer (e.g., ACK lysis buffer (Sigma)), and culturing the cells in BMDC medium (e.g., RPMI 1640 containing 10% FBS, 100 U/mL PS, 2 mM L-glutamine, 50 mM 2-mercaptoethanol, 20 ng/mL of granulocyte macrophage colony stimulating factor (GM-CSF), and 10 ng/mL of IL-4). On days 3 and 6, the BMDC medium is replaced with fresh BMDC medium. On day 8, nonadherent cells are harvested, washed two times with, e.g., phosphate-buffered saline. The resulting BMDCs may then be treated with an AIM2 inhibitor (e.g., an AIM2 inhibitory nucleic acid) and pulsed with peptide (e.g., human gp100₂₅₋₃₃ for the treatment of melanoma).

As another example, dendritic cells may be isolated from a subject using aphereresis (e.g., leukapheresis). For example, leukapheresis (e.g., using a COBE Spectra Apheresis System) may be performed on blood collected from a subject to isolate mononuclear cells. The isolated mononuclear cells are then allowed to become adherent by incubation in tissue culture flasks (e.g., for 2 hours at 37° C.). Non-adherent cells are removed by washing and adherent cells are cultured in medium supplemented with GM-CSF (e.g., 800-1000 U/mL) and interleukin-4 (e.g., 500 U/mL) for seven days. Optionally, TNF-alpha is added to the culture medium on day 5. Cells are treated with an AIM2 inhibitor (e.g., an AIM2 inhibitory nucleic acid) on day 6. Cells are then incubated with peptide antigen (e.g., human gp100₂₅₋₃₃, Wilms tumor gene 1, tyrosinase, MAGE-A3, MAGE-A2, MAGE-Al, MART-I, or NY-ESO-1 on day 8 or 9, harvested and washed for the treatment of melanoma (see, e.g., Oshita C., et al, (2012) Oncol Rep 28, 1131-8; Fukuda K., et al., (2017) Melanoma Res 27, 4, 326-34; Nowickei T S., et al., (2019) Clin Cancer Res 25, 2096-2108, each of which is incorporated by reference herein in its entirety) or tumor lysate (see, e.g., Nakai N., et al, (2006) J Dermatol 33, 462-72) before administration to the subject.

### Administration of Dendritic Cells

The dendritic cell-based cancer vaccine may be delivered to a patient or test animal by any suitable delivery route, which can include injection, infusion, inoculation, direct surgical delivery, or any combination thereof. In some embodiments, the cancer vaccine is administered to a human in the deltoid region or axillary region. For example, the vaccine is administered into the axillary region as an intradermal injection. In some embodiments, the vaccine is administered intravenously. In some embodiments, the vaccine is administered subcutaneously. In some embodiments, the vaccine is administered intratumorally.

In some embodiments, subjects administered the dendritic cells described herein are further administered other treatment(s). For example, a subject may also be administered or have received chemotherapy, radiation, one or more immune modulators (e.g., a PD-1 antagonist (e.g., an anti-PD-1 antibody) or a CTLA-4 antagonist (e.g., an anti-CTLA-4 antibody)). As another example, a subject may also be administered a PD-1 antagonist (e.g., an anti-PD-1 antibody) and adoptive T cell therapy. As another example, a subject may also be administered a CTLA-4 antagonist (e.g., an anti-CTLA-4 antibody) and adoptive T cell therapy. As another example, a subject may have also undergone or may undergo surgical therapy. Methods of treating cancer using dendritic vaccination in conjunction with chemotherapy are described in Wheeler et al., US Pat. Pub. No. 2007/0020297, which is incorporated by reference herein in its entirety.

### Compositions

The methods described herein can include the administration of pharmaceutical compositions and formulations comprising AIM2 inhibitors (e.g., inhibitory nucleic acid sequences designed to target an AIM2 RNA) described herein. Thus, provided herein are compositions (e.g., pharmaceutical compositions) comprising an AIM2 inhibitor (e.g., an AIM2 inhibitory nucleic acid) described herein (or a vector comprising same or a nucleic acid encoding same) or a dendritic cell treated with an AIM2 inhibitor as described herein.

In some embodiments, the compositions are formulated with a pharmaceutically acceptable carrier. The pharmaceutical compositions and formulations can be administered parenterally, topically, orally or by local administration, such as by aerosol or transdermally. The pharmaceutical compositions can be formulated in any way and can be administered in a variety of unit dosage forms depending upon the condition or disease and the degree of illness, the general medical condition of each patient, the resulting preferred method of administration and the like. Details on techniques for formulation and administration of pharmaceuticals are well described in the scientific and patent literature, see, e.g., Remington: The Science and Practice of Pharmacy, 21st ed., 2005. In some embodiments, the pharmaceutical compositions and formulations can be administered subcutaneously. In some embodiments, the pharmaceutical compositions and formulations can be administered intravenously. In some embodiments, the pharmaceutical compositions can be administered intratumorally.

The AIM2 inhibitors (e.g., AIM2 inhibitory nucleic acids) can be administered alone or as a component of, e.g., a vector, a cell, or a pharmaceutical formulation (composition). The AIM2 inhibitors may be formulated for administration, in any convenient way for use in human or veterinary medicine. Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Formulations of the compositions of the invention include those suitable for intravenous, subcutaneous, intratumoral, intradermal, inhalation, oral/nasal, topical, parenteral, rectal, and/or intravaginal administration. The formulations may conveniently be presented in unit dosage form and may be prepared by any methods well known in the art of pharmacy. The amount of active ingredient (e.g., nucleic acid sequences of this invention) which can be combined with a carrier material to produce a single dosage form will vary depending upon the host being treated, the particular mode of administration, e.g., intravenous, intratumoral, or subcutaneous. The amount of active ingredient which can be combined with a carrier material to produce a single dosage form will generally be that amount of the compound which produces a therapeutic effect, e.g., a reduction in tumor size.

Pharmaceutical formulations can be prepared according to any method known to the art for the manufacture of pharmaceuticals. Such drugs can contain sweetening agents, flavoring agents, coloring agents and preserving agents. A formulation can be admixtured with nontoxic pharmaceutically acceptable excipients which are suitable for manufacture. Formulations may comprise one or more diluents, emulsifiers, preservatives, buffers, excipients, etc. and may be provided in such forms as liquids, powders, emulsions, lyophilized powders, sprays, creams, lotions, controlled release formulations, tablets, pills, gels, on patches, in implants, etc.

Pharmaceutical formulations for oral administration can be formulated using pharmaceutically acceptable carriers well known in the art in appropriate and suitable dosages. Such carriers enable the pharmaceuticals to be formulated in unit dosage forms as tablets, pills, powder, dragees, capsules, liquids, lozenges, gels, syrups, slurries, suspensions, etc., suitable for ingestion by the patient. Pharmaceutical preparations for oral use can be formulated as a solid excipient, optionally grinding a resulting mixture, and processing the mixture of granules, after adding suitable additional compounds, if desired, to obtain tablets or dragee cores. Suitable solid excipients are carbohydrate or protein fillers include, e.g., sugars, including lactose, sucrose, mannitol, or sorbitol; starch from corn, wheat, rice, potato, or other plants; cellulose such as methyl cellulose, hydroxypropylmethyl-cellulose, or sodium carboxy-methylcellulose; and gums including arabic and tragacanth; and proteins, e.g., gelatin and collagen. Disintegrating or solubilizing agents may be added, such as the cross-linked polyvinyl pyrrolidone, agar, alginic acid, or a salt thereof, such as sodium alginate. Push-fit capsules can contain active agents mixed with a filler or binders such as lactose or starches, lubricants such as talc or magnesium stearate, and, optionally, stabilizers. In soft capsules, the active agents can be dissolved or suspended in suitable liquids, such as fatty oils, liquid paraffin, or liquid polyethylene glycol with or without stabilizers.

Aqueous suspensions can contain an active agent (e.g., AIM2 inhibitors, e.g., nucleic acid sequences of the disclosure) in admixture with excipients suitable for the manufacture of aqueous suspensions, e.g., for aqueous intradermal injections. Such excipients include a suspending agent, such as sodium carboxymethylcellulose, methylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

In some embodiments, oil-based pharmaceuticals are used for administration of AIM2 inhibitors (e.g., nucleic acid sequences of the disclosure). Oil-based suspensions can be formulated by suspending an active agent in a vegetable oil, such as arachis oil, olive oil, sesame oil or coconut oil, or in a mineral oil such as liquid paraffin; or a mixture of these. See e.g., U.S. Pat. No. 5,716,928 describing using essential oils or essential oil components for increasing bioavailability and reducing inter- and intra-individual variability of orally administered hydrophobic pharmaceutical compounds (see also U.S. Pat. No. 5,858,401). The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto (1997) J. Pharmacol. Exp. Ther. 281:93-102.

Pharmaceutical formulations can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent. In alternative embodiments, these injectable oil-in-water emulsions of the invention comprise a paraffin oil, a sorbitan monooleate, an ethoxylated sorbitan monooleate and/or an ethoxylated sorbitan trioleate.

The pharmaceutical compounds can also be administered by in intranasal, intraocular and intravaginal routes including suppositories, insufflation, powders and aerosol formulations (for examples of steroid inhalants, see e.g., Rohatagi (1995) J. Clin. Pharmacol. 35:1187-1193; Tjwa (1995) Ann. Allergy Asthma Immunol. 75:107-111). Suppositories formulations can be prepared by mixing the drug with a suitable non-irritating excipient which is solid at ordinary temperatures but liquid at body temperatures and will therefore melt in the body to release the drug. Such materials are cocoa butter and polyethylene glycols.

In some embodiments, the pharmaceutical compounds can be delivered transdermally, by a topical route, formulated as applicator sticks, solutions, suspensions. emulsions, gels, creams, ointments, pastes, jellies, paints, powders, and aerosols.

In some embodiments, the pharmaceutical compounds can also be delivered as microspheres for slow release in the body. For example, microspheres can be administered via intradermal injection of drug which slowly release subcutaneously; see Rao (1995) J. Biomater Sci. Polym. Ed. 7:623-645; as biodegradable and injectable gel formulations, see, e.g., Gao (1995) Pharm. Res. 12:857-863 (1995); or, as microspheres for oral administration, see, e.g., Eyles (1997) J. Pharm. Pharmacol. 49: 669-674.

In some embodiments, the pharmaceutical compounds can be parenterally administered, such as by intravenous (IV) administration or administration into a body cavity or lumen of an organ. These formulations can comprise a solution of active agent dissolved in a pharmaceutically acceptable carrier. Acceptable vehicles and solvents that can be employed are water and Ringer's solution, an isotonic sodium chloride. In addition, sterile fixed oils can be employed as a solvent or suspending medium. For this purpose any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of active agent in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated using those suitable dispersing or wetting agents and suspending agents. The sterile injectable preparation can also be a suspension in a nontoxic parenterally-acceptable diluent or solvent, such as a solution of 1,3-butanediol. The administration can be by bolus or continuous infusion (e.g., substantially uninterrupted introduction into a blood vessel for a specified period of time).

In some embodiments, the pharmaceutical compounds and formulations can be lyophilized. Stable lyophilized formulations comprising an inhibitory nucleic acid can be made by lyophilizing a solution comprising a pharmaceutical of the invention and a bulking agent, e.g., mannitol, trehalose, raffinose, and sucrose or mixtures thereof. A process for preparing a stable lyophilized formulation can include lyophilizing a solution about 2.5 mg/mL protein, about 15 mg/mL sucrose, about 19 mg/mL NaCl, and a sodium citrate buffer having a pH greater than 5.5 but less than 6.5. See, e.g., U.S. 20040028670.

The compositions and formulations can be delivered by the use of liposomes. By using liposomes, particularly where the liposome surface carries ligands specific for target cells, or are otherwise preferentially directed to a specific organ, one can focus the delivery of the active agent into target cells in vivo. See, e.g., U.S. Pat. Nos. 6,063,400; 6,007,839; Al-Muhammed (1996) J. Microencapsul. 13:293-306; Chonn (1995) Curr. Opin. Biotechnol. 6:698-708; Ostro (1989) Am. J. Hosp. Pharm. 46:1576-1587. As used in the present invention, the term “liposome” means a vesicle composed of amphiphilic lipids arranged in a bilayer or bilayers. Liposomes are unilamellar or multilamellar vesicles that have a membrane formed from a lipophilic material and an aqueous interior that contains the composition to be delivered. Cationic liposomes are positively charged liposomes that are believed to interact with negatively charged DNA molecules to form a stable complex. Liposomes that are pH-sensitive or negatively-charged are believed to entrap DNA rather than complex with it. Both cationic and noncationic liposomes have been used to deliver DNA to cells.

Liposomes can also include “sterically stabilized” liposomes, i.e., liposomes comprising one or more specialized lipids. When incorporated into liposomes, these specialized lipids result in liposomes with enhanced circulation lifetimes relative to liposomes lacking such specialized lipids. Examples of sterically stabilized liposomes are those in which part of the vesicle-forming lipid portion of the liposome comprises one or more glycolipids or is derivatized with one or more hydrophilic polymers, such as a polyethylene glycol (PEG) moiety. Liposomes and their uses are further described in U.S. Pat. No. 6,287,860.

The formulations of the invention can be administered for prophylactic and/or therapeutic treatments. In some embodiments, for therapeutic applications, compositions are administered to a subject who is need of reduced AIM2 levels, or who is at risk of or has a disorder described herein (e.g., melanoma), in an amount sufficient to cure, alleviate or partially arrest the clinical manifestations of the disorder or its complications; this can be called a therapeutically effective amount. For example, in some embodiments, pharmaceutical compositions of the invention are administered in an amount sufficient to decrease tumor sizes in the subject.

The amount of pharmaceutical composition adequate to accomplish this is a therapeutically effective dose. The dosage schedule and amounts effective for this use, i.e., the dosing regimen, will depend upon a variety of factors, including the stage of the disease or condition, the severity of the disease or condition, the general state of the patient's health, the patient's physical status, age and the like. In calculating the dosage regimen for a patient, the mode of administration also is taken into consideration.

The dosage regimen also takes into consideration pharmacokinetics parameters well known in the art, i.e., the active agents' rate of absorption, bioavailability, metabolism, clearance, and the like (see, e.g., Hidalgo-Aragones (1996) J. Steroid Biochem. Mol. Biol. 58:611-617; Groning (1996) Pharmazie 51:337-341; Fotherby (1996) Contraception 54:59-69; Johnson (1995) J. Pharm. Sci. 84:1144-1146; Rohatagi (1995) Pharmazie 50:610-613; Brophy (1983) Eur. J. Clin. Pharmacol. 24:103-108; Remington: The Science and Practice of Pharmacy, 21st ed., 2005). The state of the art allows the clinician to determine the dosage regimen for each individual patient, active agent and disease or condition treated. Guidelines provided for similar compositions used as pharmaceuticals can be used as guidance to determine the dosage regiment, i.e., dose schedule and dosage levels, administered practicing the methods of the invention are correct and appropriate.

Single or multiple administrations of formulations can be given depending on for example: the dosage and frequency as required and tolerated by the patient, the degree and amount of therapeutic effect generated after each administration (e.g., effect on tumor size or growth), and the like. The formulations should provide a sufficient quantity of active agent to effectively treat, prevent or ameliorate conditions, diseases or symptoms.

In alternative embodiments, pharmaceutical formulations for oral administration are in a daily amount of between about 1 to 100 or more mg per kilogram of body weight per day. Lower dosages can be used, in contrast to administration orally, into the blood stream, into a body cavity or into a lumen of an organ. Substantially higher dosages can be used in topical or oral administration or administering by powders, spray or inhalation. Actual methods for preparing parenterally or non-parenterally administrable formulations will be known or apparent to those skilled in the art and are described in more detail in such publications as Remington: The Science and Practice of Pharmacy, 21st ed., 2005.

Various studies have reported successful mammalian dosing using complementary nucleic acid sequences. For example, Esau C., et al., (2006) Cell Metabolism, 3 (2):87-98 reported dosing of normal mice with intraperitoneal doses of miR-122 antisense oligonucleotide ranging from 12.5 to 75 mg/kg twice weekly for 4 weeks. The mice appeared healthy and normal at the end of treatment, with no loss of body weight or reduced food intake. Plasma transaminase levels were in the normal range (AST ¾ 45, ALT ¾ 35) for all doses with the exception of the 75 mg/kg dose of miR-122 ASO, which showed a very mild increase in ALT and AST levels. They concluded that 50 mg/kg was an effective, non-toxic dose. Another study by Kriitzfeldt J., et al., (2005) Nature 438, 685-689, injected anatgomirs to silence miR-122 in mice using a total dose of 80, 160 or 240 mg per kg body weight. The highest dose resulted in a complete loss of miR-122 signal. In yet another study, locked nucleic acids (“LNAs”) were successfully applied in primates to silence miR-122. Elmen J., et al., (2008) Nature 452, 896-899, report that efficient silencing of miR-122 was achieved in primates by three doses of 10 mg kg-1 LNA-antimiR, leading to a long-lasting and reversible decrease in total plasma cholesterol without any evidence for LNA-associated toxicities or histopathological changes in the study animals.

In some embodiments, the methods described herein can include co-administration with other drugs or pharmaceuticals, e.g., other anti-cancer treatments (e.g., radiation, cytotoxic agents (e.g., chemotherapy), immunomodulatory agents (e.g., a PD-1 antagonist (e.g., an anti-PD-1 antibody) or a CTLA-4 antagonist (e.g., an anti-CTLA-4 antibody)). For example, the AIM2 inhibitors (e.g., inhibitory nucleic acids) described herein can be co-administered with drugs for treating cancer. In some embodiments, the methods described herein can include co-administration with a PD-1 antagonist (e.g., an anti-PD-1 antibody) and adoptive T cell therapy. In some embodiments, the methods described herein can include co-administration with a CTLA-4 antagonist (e.g., an anti-CTLA-4 antibody) and adoptive T cell therapy.

### Methods of Treatment

The methods described herein include methods for the treatment of cancer (e.g., melanoma). In some embodiments, the cancer is melanoma. Generally, the methods include administering a therapeutically effective amount of an AIM2 inhibitor (e.g., an AIM2 inhibitory nucleic acid) as described herein (or a vector comprising same or a nucleic acid encoding same) or a dendritic cell treated with an AIM2 inhibitor as described herein to a subject who is in need of, or who has been determined to be in need of, such treatment.

As used in this context, to “treat” means to ameliorate at least one symptom of the cancer. For example, melanoma often results in abnormal skin growths that may: be asymmetric, have an irregular or notched border, has uneven shading or dark spots, be large in diameter (e.g., greater than ¼ inch), be changing in size, shape or texture; thus, a treatment for melanoma can result in a reduction in skin growth size and a return or approach to an absence of cancerous cells in or around the skin growth. Administration of a therapeutically effective amount of a compound described herein for the treatment of a cancer will result in decreased tumor size and/or a reduction in the number of cancerous cells.

The methods of treatment described herein may be in combination with one or more additional therapies, e.g., one or more additional anti-cancer therapies. For example, the methods of treatment described herein may be performed in combination with administration to the subject: an immune checkpoint modulator (e.g., a PD-1 (programmed cell death 1) antagonist (e.g., an anti-PD-1 antibody (including those described in U.S. Pat. Nos. 8,008,449; 9,073,994; and US20110271358, pembrolizumab, nivolumab, Pidilizumab (CT-011), BGB-A317, MEDI0680, BMS-936558 (ONO-4538); anti-PDL1 (programmed cell death ligand 1) or anti-PDL2 (e.g., BMS-936559. MPDL3280A, atezolizumab, avelumab and durvalumab)) or a CTLA-4 antagonist (e.g., an anti-CTLA-4 antibody (e.g., ipilumimab or tremelimumab)), radiation, a cytotoxic agent (e.g., chemotherapy), or adoptive T cell therapy. In some instances, the methods of treatment described herein may be performed in combination with administration to the subject a PD-1 antagonist (e.g., an anti-PD-1 antibody) and adoptive T cell therapy. In some instances, the methods of treatment described herein may be performed in combination with administration to the subject a CTLA-4 antagonist (e.g., an anti-CTLA-4 antibody) and adoptive T cell therapy. Additionally or alternatively, the methods of treatment described herein may be performed in combination with an IL-1β antagonist, an IL-18 antagonist, and/or a stimulator of interferon genes (STING) agonist.

### Cell Therapy

A compound described herein for modulating, e.g., AIM2 expression, levels, or activity, e.g., an AIM2 siRNA or a polypeptide from a compound modulating AIM2 expression, level, or activity, can also be increased in a subject by introducing into a cell, e.g., a dendritic cell, a nucleotide sequence that encodes an AIM2 siRNA or a polypeptide from a compound modulating AIM2 expression, level, or activity.

The nucleotide sequence can be a nucleic acid encoding AIM2 siRNA or another polypeptide or peptide that decreases AIM2 activity, levels, or expression or an active fragment thereof, and any of: a promoter sequence, e.g., a promoter sequence from a dendritic cell gene or from another gene; an enhancer sequence, e.g., 5′ untranslated region (UTR), e.g., a 5′ UTR from a dendritic cell gene or from another gene, a 3′ UTR, e.g., a 3′ UTR from a dendritic cell gene or from another gene; a polyadenylation site; an insulator sequence; or another sequence that decreases the expression of AIM2 or of a peptide or polypeptide that decreases AIM2 expression, level, or activity. The cell (e.g., dendritic cell) can then be introduced into the subject.

Primary and secondary cells to be genetically engineered can be obtained from a variety of tissues and can include cell types that can be maintained and propagated in culture. For example, primary and secondary cells include pancreatic islet β cells, adipose cells, fibroblasts, keratinocytes, epithelial cells (e.g., mammary epithelial cells, intestinal epithelial cells), endothelial cells, glial cells, neural cells, formed elements of the blood (e.g., lymphocytes, bone marrow cells, dendritic cells, natural killer cells (Holsken, O. et al JDDG 2014, 23-28), cytotoxic T lymphocytes (Cooper, L. J. et al. Cytotherapy 2006, 8 (2):105-17), muscle cells (myoblasts) and precursors of these somatic cell types. Primary cells are preferably obtained from the individual to whom the genetically engineered primary or secondary cells will be administered. However, primary cells may be obtained from a donor (i.e., an individual other than the recipient).

The term “primary cell” includes cells present in a suspension of cells isolated from a vertebrate tissue source (prior to their being plated, i.e., attached to a tissue culture substrate such as a dish or flask), cells present in an explant derived from tissue, both of the previous types of cells plated for the first time, and cell suspensions derived from these plated cells. The term “secondary cell” or “cell strain” refers to cells at all subsequent steps in culturing. Secondary cells are cell strains which consist of primary cells which have been passaged one or more times.

Primary or secondary cells of vertebrate, particularly mammalian, origin can be transfected with an exogenous nucleic acid sequence, which includes a nucleic acid sequence encoding a signal peptide, and/or a heterologous nucleic acid sequence, e.g., encoding an AIM2 antagonist, and produce the encoded product stably and reproducibly in vitro and in vivo, over extended periods of time.

A heterologous amino acid can also be a regulatory sequence, e.g., a promoter, which causes expression, e.g., inducible expression or upregulation, of an endogenous sequence. An exogenous nucleic acid sequence can be introduced into a primary or a secondary cell by homologous recombination as described, for example, in U.S. Pat. No. 5,641,670, the contents of which are incorporated herein by reference. The transfected primary or secondary cells can also include DNA encoding a selectable marker, which confers a selectable phenotype upon them, facilitating their identification and isolation.

Vertebrate tissue can be obtained by standard methods such a punch biopsy or other surgical methods of obtaining a tissue source of the primary cell type of interest. For example, blood can be collected to obtain mononuclear cells, as a source of cells, e.g. to produce dendritic cells. A mixture of primary cells can be obtained from the tissue, using known methods, such as enzymatic digestion or explanting. If enzymatic digestion is used, enzymes such as collagenase, hyaluronidase, dispase, pronase. trypsin, elastase and chymotrypsin can be used.

The resulting primary cell mixture can be transfected directly, or it can be cultured first, removed from the culture plate and resuspended before transfection is carried out. Primary cells or secondary cells are combined with exogenous nucleic acid sequence to, e.g., stably integrate into their genomes, and treated in order to accomplish transfection. As used herein, the term “transfection” includes a variety of techniques for introducing an exogenous nucleic acid into a cell including calcium phosphate or calcium chloride precipitation, microinjection, DEAE-dextrin-mediated transfection, lipofection, electroporation or genome-editing using zinc-finger nucleases, transcription activator-like effector nuclease or the CRIPSR-Cas system, all of which are routine in the art (Kim et al (2010) Anal Bioanal Chem 397 (8): 3173-3178; Hockemeyer et al. (2011) Nat. Biotechnol. 29:731-734; Feng, Z et al. (2013) Cell Res 23 (10): 1229-1232; Jinek, M. et al. (2013) eLife 2:e00471; Wang et al (2013) Cell. 153 (4): 910-918).

Transfected primary or secondary cells undergo sufficient numbers of doubling to produce either a clonal cell strain or a heterogeneous cell strain of sufficient size to provide the therapeutic protein to an individual in effective amounts. The number of required cells in a transfected clonal heterogeneous cell strain is variable and depends on a variety of factors, including but not limited to, the use of the transfected cells, the functional level of the exogenous DNA in the transfected cells, the site of implantation of the transfected cells (for example, the number of cells that can be used is limited by the anatomical site of implantation), and the age, surface area, and clinical condition of the patient.

The transfected cells, e.g., cells produced as described herein, can be introduced into an individual to whom the product is to be delivered. Various routes of administration and various sites (e.g., renal sub capsular, subcutaneous, central nervous system (including intrathecal), intravascular, intrahepatic, intrasplanchnic, intraperitoneal (including intraomental), intramuscularly implantation) can be used. Once implanted in an individual, the transfected cells produce the product encoded by the heterologous nucleic acid or are affected by the heterologous nucleic acid itself. For example, an individual who suffers from cancer (e.g., melanoma) is a candidate for implantation of cells producing a compound described herein, e.g., an AIM2 inhibitory nucleic acid or a compound that decreases AIM2 expression, level, or activity, as described herein. Alternatively. In some embodiments, gene therapy may be used to generate AIM2-deficient DCs in a subject.

### EXAMPLES

The invention is further described in the following examples, which do not limit the scope of the invention described in the claims.

### Materials and Methods

The following materials and methods were used in the Examples set forth herein.

### Cell Culture

The murine melanoma cell line B16F10 was obtained from ATCC and the murine melanoma cell line YUMM1.7 was kindly provided by Dr. M. Bosenberg (Yale University School of Medicine, CT; now available at ATCC). B16F10 cells were cultured in DMEM (Corning) supplemented with 10% fetal bovine serum (FBS) and 100 U/ml penicillin/streptomycin (PS). YUMM1.7 cells (Meeth et al., 2016) were cultured in DMEM/F12 (Gibco) supplemented with 10% FBS, 100 U/ml PS (Corning) and 1% non-essential amino acids solution (Gibco). Both cell lines included in this example were profiled at passage 4-9 to abrogate the heterogeneity introduced by long-term culture. Both cell lines were routinely confirmed negative for Mycoplasma species by RAPIDMAP-21 (Taconic Biosciences) and were maintained at 37° C. in a humidified atmosphere of 5% CO₂.

### Mice

C57BL/6 (B6) (CD45.2) wild-type (WT), Infar^{-/-}, Cxcl10^{-/-}, Il-18^{-/-}, CD45.1 congenic B6, and Thy1.1⁺ PMEL TCR transgenic (PMEL) mice were purchased from Jackson Laboratory. Sting^{-/-} (Ishikawa and Barber, 2008) mice were kindly provided by Dr. D. Stetson (University of Washington) and backcrossed for more than 10 generations at the UMMS. Aim2^{-/-} mice of C57BL/6 background (Jones et al., 2010) were obtained from Genentech. Il-1β^{-/-} mice (Horai et al., 1998) that were backcrossed to C57BL/6 mice were kindly provided by Dr. D. Golenbock (UMMS). Aim2^{-/-} mice were intercrossed with Sting^{-/-}, Ifnar^{-/-}, or Cxcl10^{-/-} mice to produce Aim2^{-/-}Sting^{-/-}, Aim2^{-/-}Ifnar^{-/-}, and Aim2^{-/-}Cxcl10^{-/-} mice. Both male and female mice (age: 6-14 weeks) were included in the experiments, with age- and sex-matched mice used throughout.

### Generation of BMDC and Peptide Pulsed DC Vaccine

BMDCs were generated in accordance with a modified version of a method described previously (Helft et al., 2015; Lou et al., 2004; Lutz et al., 1999). Briefly, bone marrow cells isolated from the femurs and tibiae of 7M-week-old WT, Aim2^{-/-}, Aim2^{-/-}Sting^{-/-}, Aim2^{-/-}Ifnar^{-/-}, Aim2^{-/-}Cxcl10^{-/-}, Il-1β^{-/-}, and Il-18^{-/-} mice were filtered through a 70-μm nylon strainer, and red blood cells were lysed by ACK lysis buffer (Sigma Aldrich) and cultured in BMDC medium (RPMI-1640 containing 10% FBS, 100 U/mL PS, 2 mM L-glutamine (Gibco), 50 μM 2-mercaptoethanol (Sigma Aldrich), 20 ng/mL GM-CSF (PeproTech), and 10 ng/mL IL-4 (PeproTech)). The BMDC medium was replaced on days 3 and 6. On day 8, nonadherent cells were harvested, washed two times with PBS, and used for in vitro experiments. DC purity was assessed by flow cytometry to ensure staining for markers CD11c, MHC II, CD11b, and CD86 on BMDCs. For DC vaccination, nonadherent cells were pulsed for 3 hr at 37° C. with 10 μM of the human gp100₂₅₋₃₃ (hgp100) peptide (GenScript) in Opti-MEM medium (Gibco) and washed three times with PBS before their use.

### Generation of MoDC

Dendritic cells (DCs) were generated from peripheral blood mononuclear cells (PBMCs) prepared from leukopaks as previously described (McCauley et al., 2018). Briefly, to generate DCs, CD14+ mononuclear cells were isolated from PBMCs via positive selection using anti-CD14 antibody microbeads (Miltenyi). CD14+ cells were plated at density of 2×10⁶ cells/mL and cultured using RPMI-1640, supplemented with 5% heat-inactivated human AB+ serum (Omega Scientific), 1 mM sodium pyruvate, 20 mM GlutaMAX-I, 1×MEM non-essential amino acids and 25 mM HEPES pH 7.2 (RPMIHS complete) in the presence of 1:100 cytokine-conditioned media containing human GM-CSF and human IL-4 for 6 days. DC preparations were consistently >99% DC-SIGNhigh, CD11chigh, and CD14low by flow cytometry.

### Hydrophobically Modified siRNA

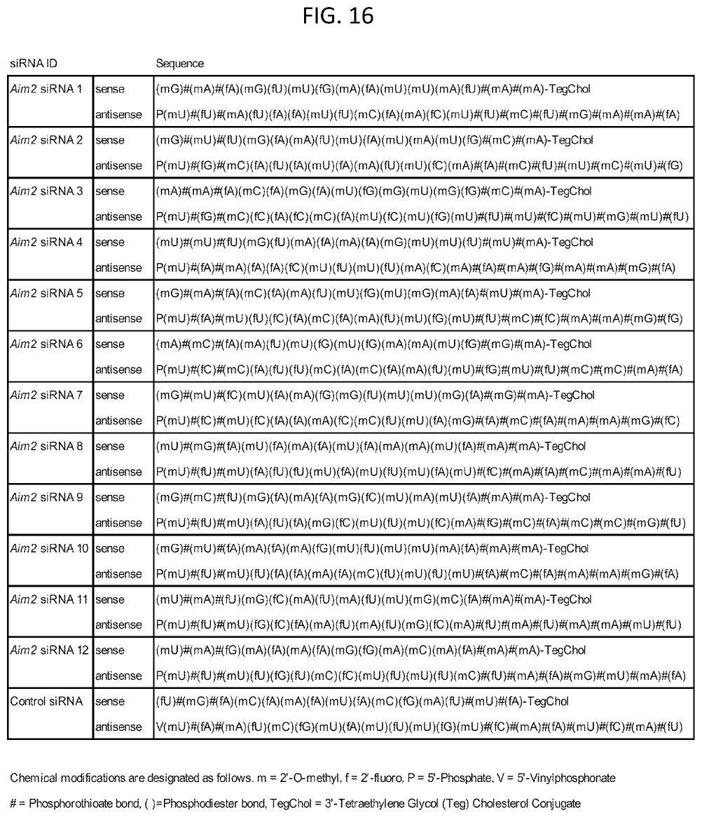

Oligonucleotides targeting Aim2 (mouse) or AIM2 (human) were chemically modified in-house as described previously to generate Aim2 and AIM2 hydrophobically modified, fully chemically stabilized siRNAs (Hassler et al., 2018). Some Aim2 siRNAs (−1 to −6) targeted the shared sequence of human and mouse AIM2 RNA and the other Aim2 siRNAs (−7 to −12) targeted the sequence of mouse AIM2 RNA. Of multiple Aim2 siRNAs, the one that showed highest (Aim2 siRNA 4) and the second highest (Aim2 siRNA 9) mouse AIM2 RNA suppression in BMDCs were used for in vitro and in vivo experiments using mouse BMDCs. Aim2 siRNAs (−2 and −4) that significantly suppressed AIM2 protein expression compared to Control siRNA in human MoDCs were used for in vitro experiments using human MoDCs. FIG. 16 lists chemical modification patterns and sequences of Aim2 siRNAs.

### Transfection of BMDCs and MoDCs With siRNA

On day 5.5 during BMDC differentiation, floating cells were collected and plated at 10.5×10⁶ cells (for in vivo experiments), 3.5×10⁵ cells (for quantitative RT-PCR analysis and ELISA), or 1.4×10⁶ cells (for Western blot analysis) in another 10-cm culture dish, 24-well plate, or 6-well plate, respectively. On day 6, DC medium was replaced with RPMI-1640 containing siRNA (35 nM) complexed with GeneSilencer Transfection Reagent (7 μl/ml; Genlantis) and incubated for 4 hr. Subsequently, RPMI-1640, FBS, L-glutamine, 2-mercaptoethanol, GM-CSF, and IL-4 were added to the medium to create RPMI-1640 supplemented with 3% FBS, 2 mM L-glutamine, 50 μM 2-mercaptoethanol, 20 ng/mL GM-CSF, and 10 ng/mL IL-4. Forty-eight hours later, cells were harvested, washed twice with PBS, and used for quantitative RT-PCR analysis, Western blot analysis, ELISA, or generating hgp100 peptide-pulsed DC vaccine. In some experiments, the medium of siRNA-transfected BMDCs was replaced with fresh BMDC medium every other day from 48 h later transfection and harvested 3, 10, or 22 days after transfection to perform RT-PCR analysis.

Similar to BMDC, on day 6 during MoDC differentiation, floating cells were collected and plated at 3.5×10⁵ cells (for Western blot analysis and ELISA) in another 24-well plate and cultured in RPMI-1640 containing siRNA (35 nM) complexed with GeneSilencer Transfection Reagent (7 μl/ml) and incubated for 4 hr. Subsequently, RPMI-1640, FBS, L-glutamine, and 2-mercaptoethanol were added to the medium to create RPMI-1640 supplemented with 3% FBS, 2 mM L-glutamine, and 50 μM 2-mercaptoethanol. Forty-eight hours later, MoDCs were harvested, then left untreated for 6 h (non-primed), or then primed for 6 h with LAS at a final concentration of 1 μg/ml, and used for Western blot analysis and ELISA.

### Tumor Models

B16F10 and YUMM1.7 melanoma cells (1.0×10⁶) were resuspended in 100 μL of PBS, and implanted subcutaneously into the right flank of 6-12-week-old WT and Aim2^{-/-} mice. To examine tumor growth, the tumor size was measured in two dimensions by caliper and is expressed as the product of two perpendicular diameters.

Mice were euthanized on indicated days in the FIGs. or if the tumor ulcerated. For all treatment experiments, mice were randomized for different treatments when the tumors were palpable. The combination of ACT and DC vaccination was performed according to a modified version of a previously described method (Lou et al., 2004; Rashighi et al., 2014). PMELs were isolated from the spleens of PMEL mice through negative selection on microbeads (Miltenyi Biotec) according to the manufacturer's instructions. After 7 days of tumor injection, purified PMELs (1.0×10⁶) and the whole bulk of cultured BMDCs pulsed with hgp100 peptide were injected intravenously into sublethally irradiated (500 rad, day −1) WT mice (Day 0). The number of BMDCs was normalized to contain 1.0×10⁶ hgp100 peptide-pulsed CD11c⁺MHCII⁺ BMDCs to avoid the interexperimental variability of DC vaccination because of subtle differences in DC purity. Then, recombinant mouse IL-2 was administered intraperitoneally (6×10⁴ units) once daily for 3 consecutive days from 1 day after vaccination. In experiments to track vaccinated DCs, B6 CD45.1 hosts were used instead of WT mice. In some experiments, 50 μL of DNase I (Invitrogen; 1000 U/mL) or 50 μL of PBS was administered intratumorally every other day from 2 to 18 days after vaccination. For anti-PD-1 treatment experiments, WT mice were administered 250 mg of anti-PD-1 antibody (clone RMP1-14; BioXCell) or 250 mg of control isotype-matched Ab (clone 2A3; BioXCell) intraperitoneally on days 5, 8, 11, and 14. Furthermore, some WT mice were given DC vaccination intravenously on day 5, or the combination of DC vaccination and anti-PD-1 Ab on day 5 followed by anti-PD-1 Ab on days 8, 11, and 14 after B16F10 inoculation.

### Flow Cytometry

Tumor, tumor draining inguinal lymph nodes, and spleen were harvested at the indicated times. Draining lymph nodes and spleen were disrupted by 3-ml plunger and cell suspensions were passed through 100-μm filters. The resected mouse tumor was minced with a razor blade and digested with collagenase D (1 mg/ml Roche) and deoxyribonuclease I (0.5 mg/ml; Sigma-Aldrich) for 30 min in a 37° C. shaking incubator (75 rpm). After enzymatic dissociation, the sample was transferred to the ice to stop the reaction and filtered through a 70 μm cell strainer. Red blood cells in the cell suspensions from tumor and spleen were lysed with ACK lysis buffer followed by washing with the FACS buffer. The samples were then resuspended in the FACS buffer. Cell suspensions were blocked with Fc block 2.4G2 (Bio X Cell) and stained with LIVE/DEAD Blue (1:1000; Invitrogen) and relevant surface antibodies at 4° C. for 45 minutes. Subsequently, cells were washed two times and fixed with Cytofix/Cytoperm solution (BD Biosciences). For intracellular staining, relevant antibodies diluted in Perm/Wash Buffer (BD Biosciences) were applied to fixed cells and allowed to incubate for 30 minutes. Intracellular staining of FoxP3 was done with the use of FoxP3/Transcription Factor Staining kit (eBioscience) after surface staining. For intracellular cytokine staining, cells were stimulated with 12-myristate 13-acetate (PMA) (50 ng/ml, Sigma-Aldrich) and ionomycin (1 μg/ml, Sigma-Aldrich) in the presence of Brefeldin A (Biolegend) for 4 hours before staining with antibodies against cell surface molecules. After staining steps, cells were washed twice with FACS buffer. Data were collected with an LSR II and were analyzed with FlowJo software. In some experiments, the CountBright Absolute Counting Beads (Thermo Fischer Scientific) were added to the samples in order to quantify the absolute DC number in each sample.

Antibodies used: antibodies specific to CD45 (30-F11), CD45.1 (A20). CD45.2 (104), CD3 (17A2), CD4 (RM4-5), CD8a (53-6.7), Thy1.1 (OX-7), CD11c (N418), CD11b (M1/70), F4/80 (BM8), MHCII (I-A/I-E) (M5/114.15.2), TNFα (MP6-XT22), and IFNγ (XMG1.2) (Biolegend); antibody specific to CD86 (GL-1) (Tonbo Biosciences); antibody specific to FoxP3 (FJK-16s) (eBioscience). These specific antibodies were used for flow cytometry analysis and fluorescence minus one (FMO) controls were used to assist in gating.

### Purification of Tumor-Derived DNA and Stimulation of BMDCs and MoDCs

Genomic DNA from B16F10 melanoma cells (B16F10 DNA) and human melanoma xenograft (melanoma DNA) was purified using the DNeasy Blood & Tissue Kit (Qiagen), following the manufacturer's instructions.

Human melanoma xenograft was established from the surgical specimen of primary tumor of one melanoma patient at the UMMS. Briefly, the patient-derived melanoma was minced and loaded into 1-cc syringes with 14-gauge needles. Subsequently, the tumor piece was inoculated subcutaneously at the right flank of NSG mice. After the mice developed the tumor of approximately 10×10×10 mm size, tumor was removed and the portion of tumor was minced and used to extract melanoma DNA.

BMDCs were plated at 3.5×10⁵ cells in 24-well plates (for quantitative RT-PCR analysis and ELISA) or 1.4×10⁶ cells in 6-well plates (for Western blot analysis) and transfected with OPTI-MEM medium containing B16F10 DNA (0.1 or 1 μg/ml) complexed with Lipofectamine 2000 (1 μl/ml; Invitrogen). Similarly, MoDCs were plated at 3.5×10⁵ cells in 24-well plates (for ELISA and western blot analysis) and transfected with OPTI-MEM medium containing melanoma DNA (1 μg/ml) complexed with Lipofectamine 2000 (1 μl/ml).

### ELISA

Tumor tissues were homogenized in T-PER Tissue Protein Extraction Reagent (Thermo Scientific) supplemented with complete EDTA-free protease-inhibitor (Roche) and phosphatase inhibitor (PhosSTOP. Roche). Cell culture supernatants were obtained from BMDCs stimulated by B16F10-derived DNA for 4 hr (IFN-β) or 10 hr (CXCL10, IL-1β, and IL-18) and from siRNA-transfected LPS-primed MoDCs stimulated by human melanoma-derived DNA for 12 hr (IFN-β, CXCL10, IL-1β, and IL-18). The amount of IFN-β in tumor lysate was measured with Mouse IFN Beta ELISA Kit, High Sensitivity (PBL Assay Science) according to the manufacturer's instructions. The concentration of IFN-β, CXCL10, IL-1β, and IL-18 in supernatants from BMDCs stimulated with B16F10-derived DNA were assessed using Mouse IFN-β Duoset ELISA, Mouse CXCL10 Duoset ELISA, Mouse CXCL10 Duoset ELISA (all R&D systems), and Mouse IL-18 ELISA Kit (Abcam) according to the manufacturer's instructions, respectively. The concentration of human IFN-β, CXCL10, IL-1β, and IL-18 in supernatants from siRNA-transfected LPS-primed MoDCs stimulated with human-melanoma derived DNA were assessed using human IFN-β Duoset ELISA, human CXCL10 Duoset ELISA, human CXCL10 Duoset ELISA, and human IL-18 Duoset ELISA Kit (all R&D systems) according to the manufacturer's instructions, respectively.

### Quantitative RT-PCR Analysis

Total RNA of Mock-, Control siRNA-, or Aim2 siRNA-transfected WT BMDCs 2, 10, and 22 days after transfection and BMDCs stimulated with B16F10 DNA for 4 hr were extracted with the use of a RNeasy Mini Kit (Qiagen). The RNA isolated from BMDCs, was subjected to reverse transcription with the use of iScript cDNA synthesis kit (Bio-Rad) followed by quantitative PCR analysis with the use of iQ SYBR Green Supermix (Bio-Rad) in an iCycler iQ (Bio-Rad), as previously described (Rashighi et al., 2014). Gene expression was normalized by the corresponding amount of β-actin mRNA.

The sequences of the PCR primers (forward and reverse, respectively) were as follows:

mouse Ifnb46,
(SEQ ID NO: 33)
5′-ATAAGCAGCTCCAGCTCCAA-3′,
(SEQ ID NO: 34)
5′-CTGTCTGCTGGTGGAGTTCA-3′;
mouse Ifna47,
(SEQ ID NO: 35)
5′-ATGGCTAGGCTCTGTGCTTTCCT-3′,
(SEQ ID NO: 36)
5′-AGGGCTCTCCAGACTTCTGCTCTG-3′;
mouse Cxcl1045,
(SEQ ID NO: 37)
5′-AGGGGAGTGATGGAGAGAGG-3′,
(SEQ ID NO: 38)
5′-TGAAAGCGTTTAGCCAAAAAAGG-3′;
mouse Cxcl945,
(SEQ ID NO: 39)
5′-ATCTCCGTTCTTCAGTGTAGCAATG-3′,
(SEQ ID NO: 40)
5′-ACAAATCCCTCAAAGACCTCAAACAG-3′;
mouse Aim248,
(SEQ ID NO: 41)
5′-GTTGAATCTAACCACGAAGTCC-3′,
(SEQ ID NO: 42)
5′-CTACAAGGTCCAGATTTCAACTG-3′;
mouse Actb45,
(SEQ ID NO: 43)
5′-GGCTGTATTCCCCTCCATCG-3′,
(SEQ ID NO: 44)
5′-CCAGTTGGTAACAATGCCATGT-3′.

### Western Blot Analysis

Mouse BMDCs or human MoDCs were lysed in RIPA buffer (Thermo Scientific) supplemented with complete EDTA-free protease inhibitor and phosphatase inhibitor. The lysates were incubated for 30 min on ice and centrifuged at 15,000×g for 20 min at 4° C. The supernatants were denatured at 70° C. for 10 min in NuPAGE LDS sample buffer (Life Technologies) with NuPAGE sample reducing agent (Life Technologies). Samples were separated by MOPS-SDS Running Buffer (Life Technologies) and proteins were transferred onto a PDVF membrane (Merck Millipore). Membranes were blocked in TBS-T containing 4% nonfat dry milk for 2 h at room temperature followed by overnight incubation with anti-TBKJ (D1B4), anti-pTBK1 (D52C2), anti-IRF3 (D83B9), anti-pIRF3 (4D4G), anti-mouse AIM2, anti-human AIM2 (D5X7K), or anti-Vinculin Ab (all from Cell Signaling Technology) primary antibody at 4° C. Immune complexes were detected with anti-rabbit IgG, HRP-linked secondary antibody (Cell Signaling Technology), ECL Prime Western Blotting Detection Reagents (GE Healthcare), and a LAS-4000 instrument (Fujifilm).

### Immunohistofluorescence Staining of Human Melanoma and Quantification

Cases were selected randomly after approval by the Surveillance Committee for Human Subjects Research at the School of Medicine of Keio University. This study involved 31 patients treated at the Department of Dermatology of Keio University Hospital from 1995 to 2013. Diagnoses of malignant melanoma were confirmed histologically, and the study was designed to evaluate tumor thickness in primary lesions. The total follow-up period was 3-195 months.

Tissue was fixed with 4% paraformaldehyde, embedded in paraffin, and sectioned at a thickness of 4 μm. Sections were paraffin-depleted and rehydrated in a graded series of ethanol solutions. Sections were subjected to 10 min of microwave treatment in citrate buffer (pH 6.0) and were allowed to cool at room temperature. Non-specific binding was blocked in 1% goat serum for 30 min at room temperature, and sections were incubated with the following primary antibodies (Supplemental Table 3) diluted in PBS-T: rabbit anti-AIM2 polyclonal antibody (eBioscience) (1:800) and mouse anti-CD11c monoclonal antibody (Proteintech) (1:200) overnight at 4° C. After washing, binding of the AIM2 antibodies was visualized with Alexa Fluor 568-labeled goat antibodies to rabbit IgG (Invitrogen) and that of those to CD11c was visualized with Alexa Fluor 488-labeled goat antibodies to mouse IgG (Invitrogen), and sections were mounted in VECTASHIELD HardSet Antifade Mounting Medium with DAPI (Vector laboratories). The uniformity of staining was confirmed each time by comparing stained samples with the subcutaneous fat tissue. Images were observed using the Zeiss Axio Observer (Carl Zeiss), collected with the Axiovision software (ver. 4.8).

To evaluate stained samples, the focus of greatest inflammation was identified on each slide and at least 5 contiguous 20× high-power fields (HPF) images were collected. Then, CD11c⁺ or AIM2⁺CD11c⁺ cells infiltrating the tumor were quantified and averaged to estimate cell counts of those cells. The investigator was blinded while assessing the infiltration of AIM2 expressing DCs infiltrated in human melanoma samples.

### Statistical Analysis

Values are expressed as mean±SEM. Dual comparisons were made by Mann-Whitney's test, and groups of three or more were compared by one-way ANOVA with Tukey's or Dunnett's multiple-comparison test (for unpaired), or Friedman (for paired data) tests with Dunn's multiple comparison test. For tumor growth curve analysis, two-way ANOVA was performed with Sidak's or Tukey's multiple-comparison test (for unpaired). Statistically significant differences are indicated as follows: *p<0.05, **p<0.01, ***p<0.001, ****p<0.0001. GraphPad Prism7 software was used to perform analyses.

### Study Approval

All mice were housed in pathogen-free facilities at the UMMS, and procedures were approved under protocol #2266 by the UMMS Institutional Animal Care and Use Committee and in accordance with the NIH guidelines. Human blood (Leukopaks) were obtained from anonymous, healthy blood donors (New York Biologics). As per NIH guidelines (grants.nih.gov/grants/policy/hs/faqs_aps_definitions.htm), experiments with these cells were declared non-human subjects research by the UMMS Institutional Review Board (IRB). Human melanoma samples were collected from patients examined by a dermatologist at University of Massachusetts Medical School (UMMS) and Keio University School of Medicine. The patients analyzed in this study were diagnosed with cutaneous melanoma and gave informed consent before study inclusion. Patient studies and human sample collection were performed according to protocols approved by the IRB of UMMS and Keio University School of Medicine.

### Example 1: AIM2 Inhibitory Nucleic Acids

siRNAs were designed to specifically target AIM2. To this end, the mouse and human AIM2 mRNA sequences were evaluated for areas of conservation to design siRNAs that target both mouse and human AIM2. Table 1, above, provides the AIM2 mRNA sequence targets for the exemplary Aim2 siRNA duplexes. Table 1, above, also provides the sequences (in modified and unmodified forms) of the exemplary Aim2 siRNA duplex RNA molecules. The modified siRNA duplexes of Table 1 were tested for their ability to suppress mouse AIM2 gene expression. Based on the average of five separate experiments, each of the modified siRNA duplexes of Table 1 resulted in less than 0.45 relative AIM2 gene expression (normalized to actin) (see Table 2).

TABLE 2

| | Experi- | Experi- | Experi- | Experi- | Experi- | |
|---|---|---|---|---|---|---|
| | ment 1 | ment 2 | ment 3 | ment 4 | ment 5 | Average |
| mock | 1.00000 | 1.00000 | 1.00000 | 1.00000 | 1.00000 | 1.000000 |
| Aim2 | 0.15543 | 0.20117 | 0.14193 | 0.77990 | 0.74788 | 0.405262 |
| siRNA 2 | | | | | | |
| Aim2 | 0.20417 | 0.16423 | 0.22118 | 0.42797 | 0.44703 | 0.292916 |
| siRNA 4 | | | | | | |
| Aim2 | 0.06656 | 0.54080 | 0.19290 | 0.40486 | 0.30128 | 0.301279 |
| siRNA 9 | | | | | | |

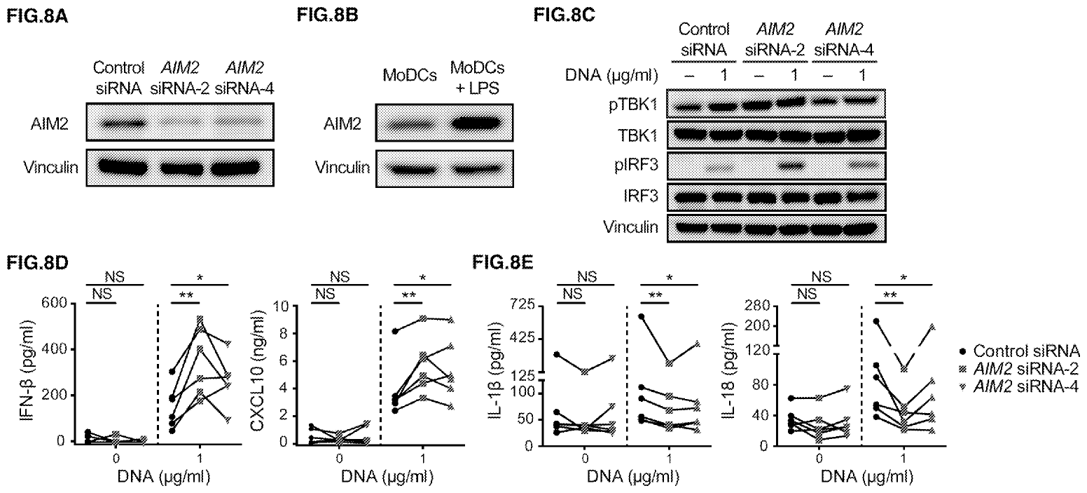

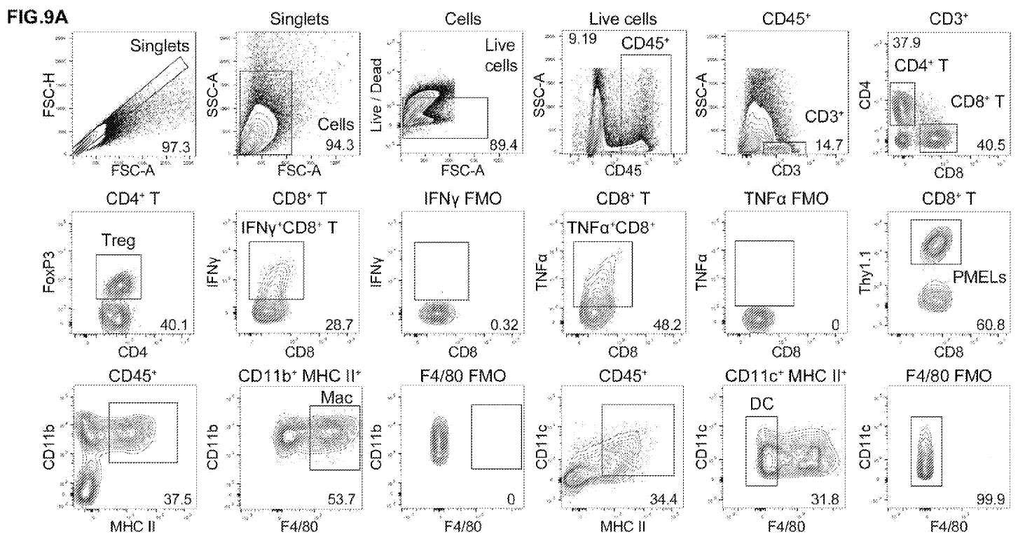

Western blot analysis for human AIM2 protein in the lysates of human MoDCs transfected with Control siRNA and Aim2 siRNA (Aim2 siRNA no 2 or no 4) confirmed that human MoDCs transfected with Aim2 siRNA (Aim2 siRNA no 2 or no 4) showed markedly lower protein expression of AIM2 than Control siRNA-transfected human MoDCs (FIG. 8A).

### Example 2: AIM2 Regulates Anti-Tumor Immunity and Serves as a Viable Therapeutic Target for Melanoma Immunotherapy

### Introduction

This example shows that AIM2 expression correlates with tumor progression in human melanoma patients and functions as a negative regulator of the STING pathway within tumor infiltrating DCs after vaccination through ACT. The density and proportion of AIM2-expressing TIDCs correlates with both the thickness and stage of melanoma. AIM2 suppresses STING-type I IFN signaling and promotes IL-1β and IL-18 secretion in response to tumor-derived DNA. Eliminating AIM2 signaling during DC vaccination by using either Aim2-deficient (Aim2^{-/-}) BMDCs or siRNA-mediated knockdown of Aim2 prior to treatment improved the efficacy of both ACT and anti-PD-1 immunotherapy. Antigen-loaded Aim2^{-/-} DC vaccine migrated to the tumor and promoted CD8⁺ T cell infiltration through the production of CXCL10, while limiting accumulation of regulatory T cells, thus making “cold” tumors “hot”. This effect required STING type I IFN signaling, and was only partially recapitulated using Il1β^{-/-} or Il18^{-/-} DC vaccines. Furthermore, AIM2 siRNA-transfected human monocyte-derived DCs stimulated with tumor-derived DNA demonstrated an increased inflammatory response, similar to mouse Aim2^{-/-} BMDCs. In summary, without being bound by any particular theory, AIM2 siRNA-transfected DC vaccination represents an effective strategy to improve the efficacy of melanoma immunotherapy (e.g., in melanoma) by promoting STING-induced IFN secretion, as well as limiting IL-1β and IL-18 production.

### Results

### AIM2 Restricts Anti-Melanoma Immunity Within the Melanoma Microenvironment

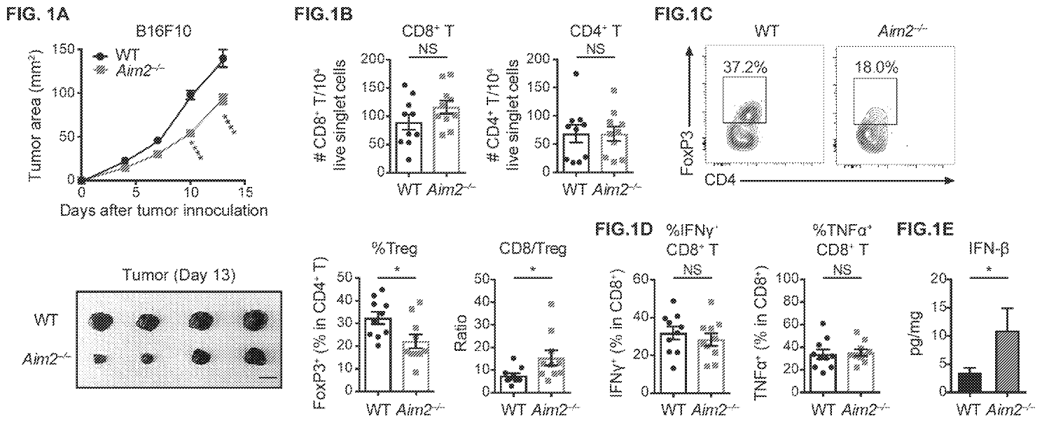

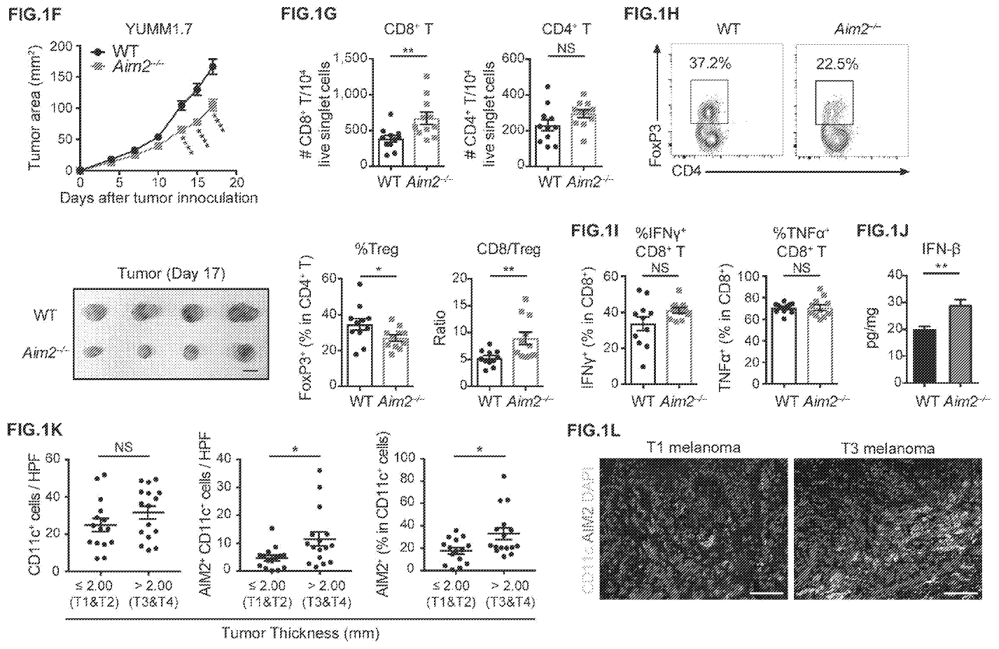

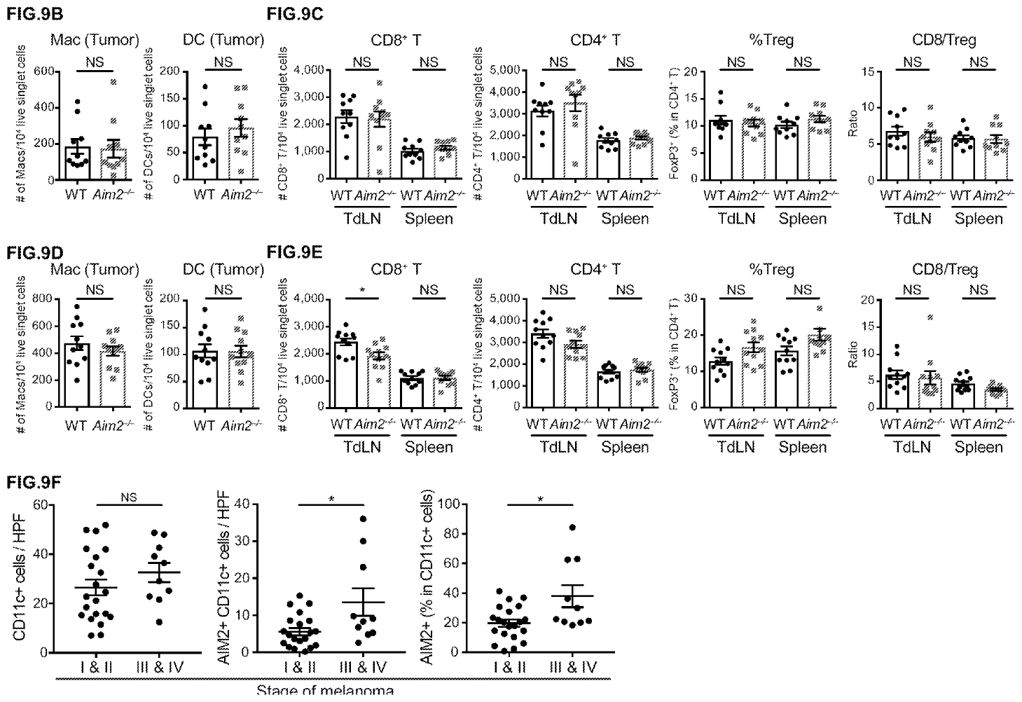

To determine whether AIM2 regulates melanoma progression, we subcutaneously challenged wild-type (WT) and Aim2^{-/-} mice with B16F10, a poorly immunogenic melanoma cell line that is resistant to anti-PD-1 Ab therapy (Homet Moreno et al., 2016). We found that Aim2^{-/-} mice exhibited significantly slower tumor growth than WT mice (FIG. 1A). Within the tumor, numbers of CD8⁺ or CD4⁺ T cells, macrophages (MACs), or DCs did not differ between WT and Aim2^{-/-} mice, whereas Aim2^{-/-} mice had a significantly smaller proportion of regulatory T cells (Tregs) and resulting higher CD8/Treg ratio compared to WT mice (FIGS. 1B, 1C, and 9B). Numbers of CD8⁺ or CD4⁺ T cells, proportion of Tregs, CD8/Treg ratio in the tumor draining lymph node (TdLN) or spleen also did not differ between WT and Aim2^{-/-} mice (fig. S1C). To test whether cytokines known to support anti-tumor immunity are induced in Aim2^{-/-} mice, we measured the percentage of IFN-γ or TNF-α producing CD8⁺ T cells and IFN-β concentration in the tumor. There was no difference in the percentage of IFN-γ or TNF-α producing CD8⁺ T cells within the tumor (FIG. 1D), however the B16F10 tumor in Aim2^{-/-} mice had a significantly higher amount of IFN-β protein compared to that of WT mice (FIG. 1E). These results suggested that AIM2 plays an immunosuppressive role within the melanoma microenvironment.

Similarly, another poorly immunogenic melanoma cell line YUMM1.7 (Homet Moreno et al., 2016), grew more slowly in Aim2^{-/-} mice than in WT mice (FIG. 1F). Aim2^{-/-} mice had significantly higher numbers of CD8⁺ T cells, fewer proportion of Tregs, and higher CD8/Treg ratio in the tumor than WT mice, whereas there was no difference in the numbers of CD4⁺ T cells, MACs, or DCs (FIGS. 1G, 1H, and 9D). In contrast to CD8⁺ Tumor-infiltrating lymphocytes (TILs), the numbers of CD8⁺ T cells in TdLN were significantly lower in Aim2^{-/-} mice than in WT mice whereas there was no difference in the spleen. There was also no difference in the numbers of CD4⁺ T cells, proportion of Tregs, CD8/Treg ratio in the TdLN or spleen between WT and Aim2^{-/-} mice (FIG. 9E). Furthermore, there was no difference in the percentage of IFN-γ or TNF-α producing CD8⁺ TILs between WT and Aim2^{-/-} mice (FIG. 1I). However, the YUMM1.7 tumor in Aim2^{-/-} mice had a significantly higher amount of IFN-β protein than that of WT mice (FIG. 1J), similar to B16F10 melanomas reported above. These results suggested that the immunosuppressive effect of AIM2 in melanoma microenvironment is not limited to the B16F10 model.

### AIM2 Expression in Human Melanoma Infiltrating DCs Correlates With Tumor Progression

In melanoma, TIDCs are the major producers of IFN-β (Deng et al., 2014), and we found that Aim2^{-/-} mice had significantly greater amounts of IFN-β in implanted melanoma compared to those of WT mice. Therefore, we next addressed whether AIM2 is expressed in DCs infiltrating human melanoma tissue and whether AIM2 expression correlates with tumor progression. To test this, we quantified the expression of AIM2 and CD11c on histological sections of primary lesions of 31 melanoma patients. Although the density of CD11c+cells were similar between thin (≤2.00 mm, T1 and T2) and thick (>2.00 mm, T3 and T4) cutaneous melanoma, thick melanomas had a higher density and proportion of AIM2-expressing CD11c+ cells compared to thin melanoma (FIGS. 1K and 1L). Similarly, primary lesions of advanced melanoma patients (stage III and IV) had a higher density and proportion of AIM2-expressing CD11c+ cells compared to those of melanoma patients without metastasis (stage I and II) (FIG. 9F). These findings indicate that AIM2-expressing TIDCs are increased in patients with melanoma and correlate with tumor progression.

### DC Vaccination is Enhanced by AIM2-Deficient DCs, which is Mediated by STING-type I IFN Signaling

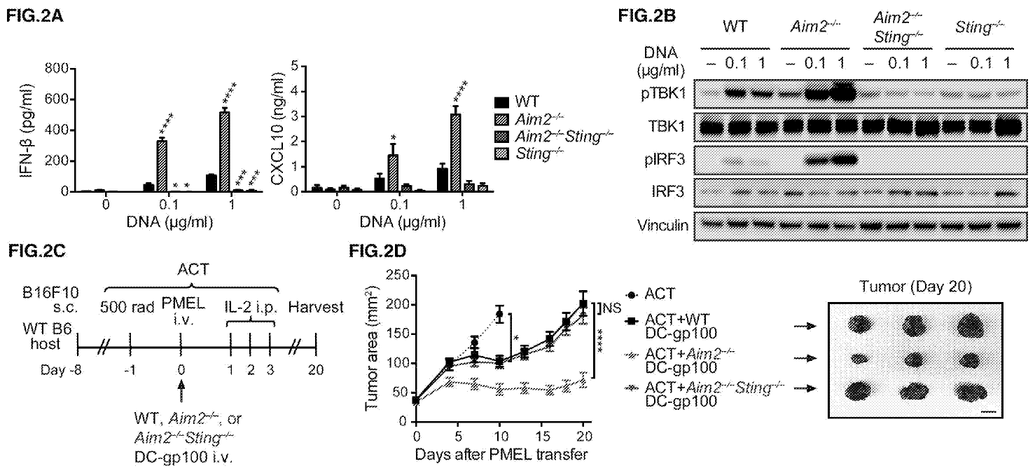

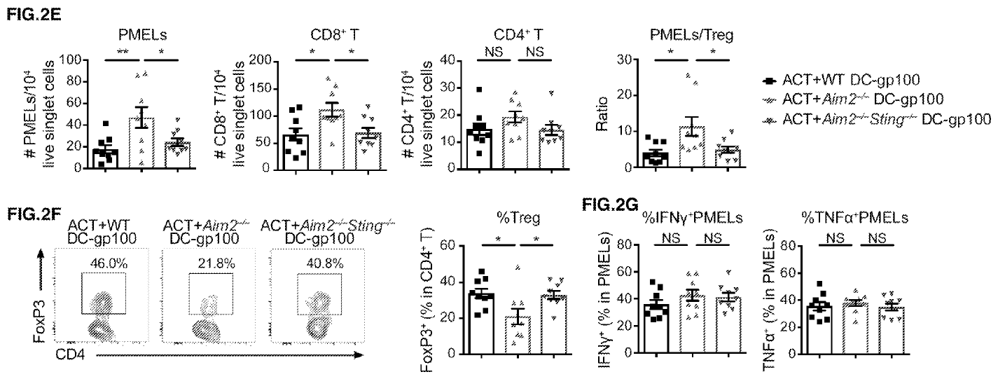

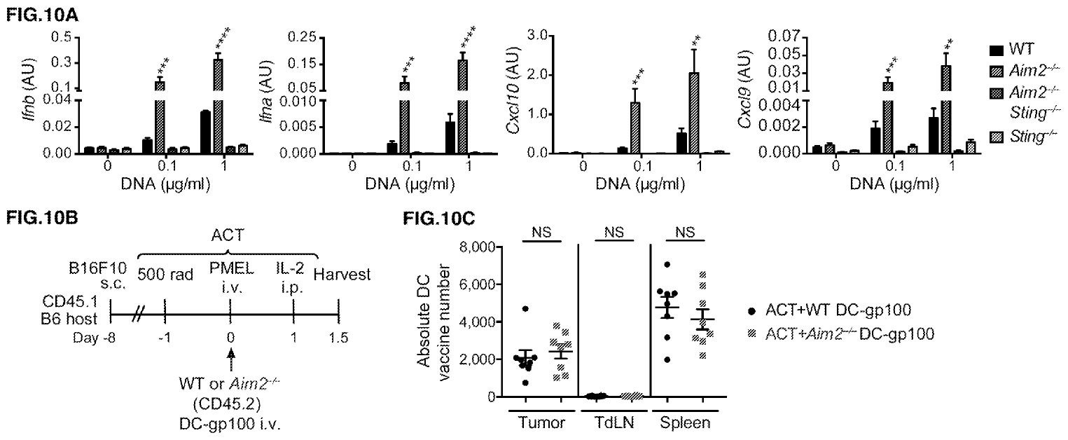

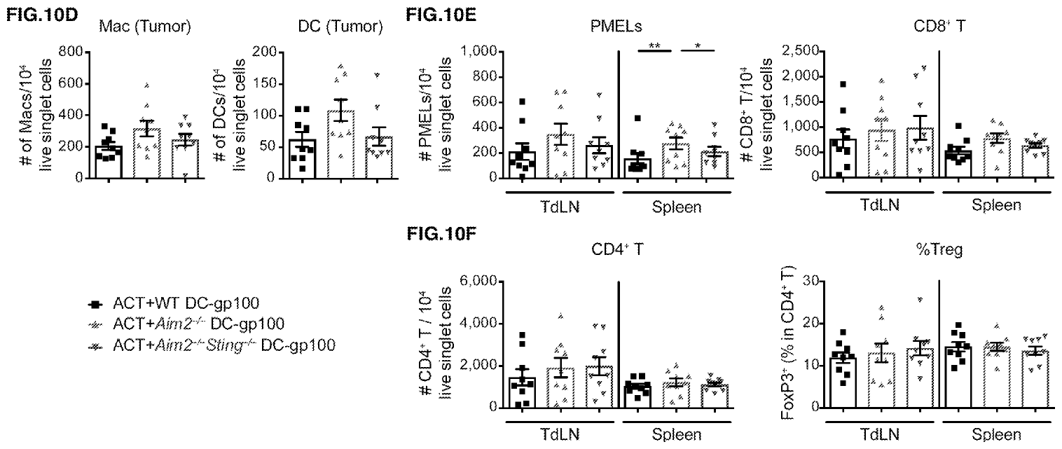

Since tumor-derived cytosolic DNA is known to activate the cGAS-STING pathway to produce type I IFN in TIDCs, we examined the role of AIM2 in controlling these responses in vitro by stimulating BMDCs with B16F10-derived DNA (B16F10 DNA), delivered via lipofection. The levels of mRNA for IFN-β and IFN-α as well as the IFN-regulated chemokines CXCL10 and CXCL9 were all significantly increased in Aim2^{-/-} BMDCs compared with those in WT BMDCs in response to B16F10 DNA. Furthermore, in agreement with previous studies (Corrales et al., 2016; Banerjee et al., 2018), WT and Aim2^{-/-} BMDCs induced IFN-β and CXCL10 production in a dose-dependent manner and Aim2^{-/-} BMDCs secreted significantly more IFN-β and CXCL10 than WT BMDCs following stimulation with B16F10 DNA. These responses were all abolished in Aim2^{-/-}Sting^{-/-} BMDCs and Sting^{-/-} BMDCs, indicating that AIM2 in BMDCs inhibits the production of type I IFN and interferon stimulated gene products in response to tumor-derived DNA through STING (FIGS. 2A and 10A), consistent with earlier observations (Banerjee et al., 2018; Rathinam et al., 2010). Indeed, following stimulation with B16F10 DNA, Aim2^{-/-} BMDCs showed enhanced phosphorylation of TBK1 (pTBK1) and IRF3 (pIRF3), proteins downstream of STING-type I IFN signaling, compared to WT BMDCs. These responses were abolished in Aim2^{-/-}Sting^{-/-} and Sting^{-/-} BMDCs, suggesting that AIM2 inhibits STING-type I IFN signaling in response to tumor-derived DNA in BMDCs (FIG. 2B).

Given the enhanced activation of STING-type I IFN signaling in Aim2^{-/-} BMDCs in response to tumor-derived DNA, we next examined the functional role of AIM2 in DCs during ACT in vivo. To evaluate whether Aim2^{-/-} DC vaccination can be used to enhance the anti-melanoma immunity of immunotherapies, we administered hgp100 peptide-pulsed BMDCs (DC-gp100) with ACT, a combination therapy of radiation, IL-2, and adoptively transferred T cells. The T cells were transgenic for Thy1.1, as well as a T cell receptor (TCR) that recognizes gp100 (also called premelanosome protein or PMEL), a tumor-specific antigen in B16F10 melanoma (FIG. 2C and FIG. 10B). Using CD45.1 B6 mice as hosts, we observed that intravenously injected DCs (CD11c⁺ MHCII⁺ Thy1.1⁻CD45.2⁺ cells) migrate into the tumor, TdLN, and the spleen within 1.5 days after injection, with the highest number in the spleen, and this was unaffected by AIM2 deficiency (FIGS. 10B and 10C).

Consistent with previous reports (Lou et al., 2004), the combination of DC vaccination with ACT led to a more robust antitumor response than ACT alone. Among mice receiving ACT with DC-gp100, those receiving Aim2^{-/-} DCs-gp100 exhibited significantly lower tumor burden than WT DC-gp100 and Aim2^{-/-}Sting^{-/-} DC-gp100 (FIG. 2D). Within the tumor, hosts receiving Aim2^{-/-} DC-gp100 had significantly higher numbers of PMELs, CD8⁺ T cells, a decreased proportion of Tregs, and higher PMEL/Treg ratio in the tumor than those receiving WT DC-gp100 and Aim2^{-/-}Sting^{-/-} DC-gp100, whereas there was no difference in the numbers of CD4⁺ T cells, MACs, and DCs among the groups (FIGS. 2E, 2F, and 10D). Furthermore, there were significantly more PMELs in the spleen in hosts receiving Aim2^{-/-} DC-gp100 (FIG. 10E). In contrast, the number of PMELs in the TdLN, numbers of CD8⁺ and CD4⁺ T cells, and proportion of Tregs in the TdLN and spleen did not differ among the three groups (FIGS. 10E and 10F). Furthermore, there was no difference in the percentage of IFN-γ or TNF-α producing PMELs within the tumor among three groups (FIG. 2G). Together, these results suggest that Aim2^{-/-} DC vaccination improves the efficacy of ACT, and the enhanced anti-melanoma immunity of Aim2^{-/-} DC vaccine is dependent on STING signaling.

### Enhanced Anti-Melanoma Immunity of AIM2-Deficient DC Vaccination Depends on the Recognition of Tumor-Derived DNA, but not Suppression of Pyroptosis

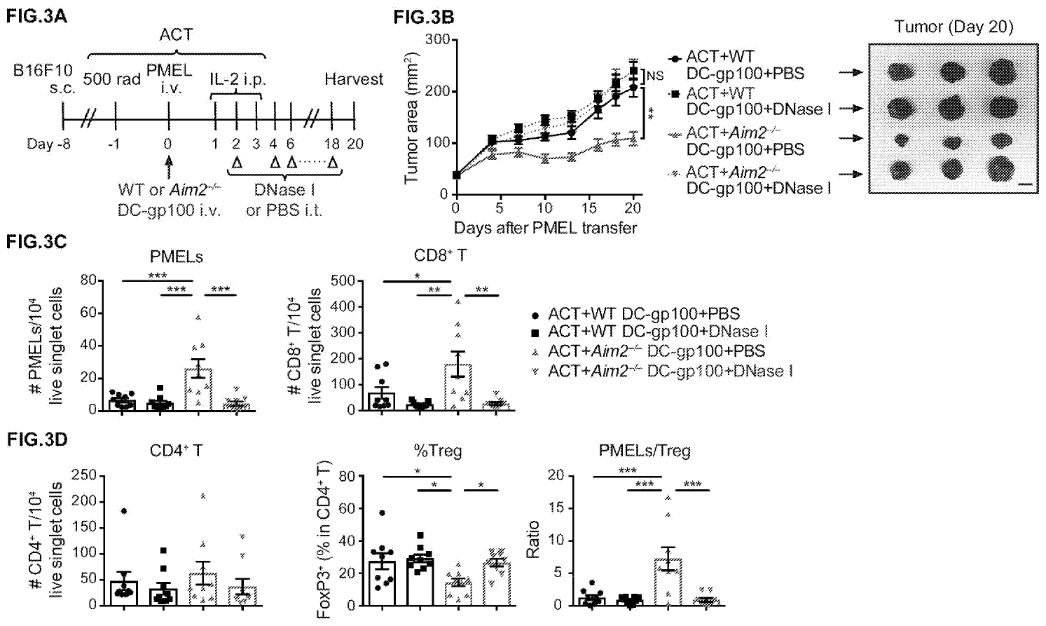

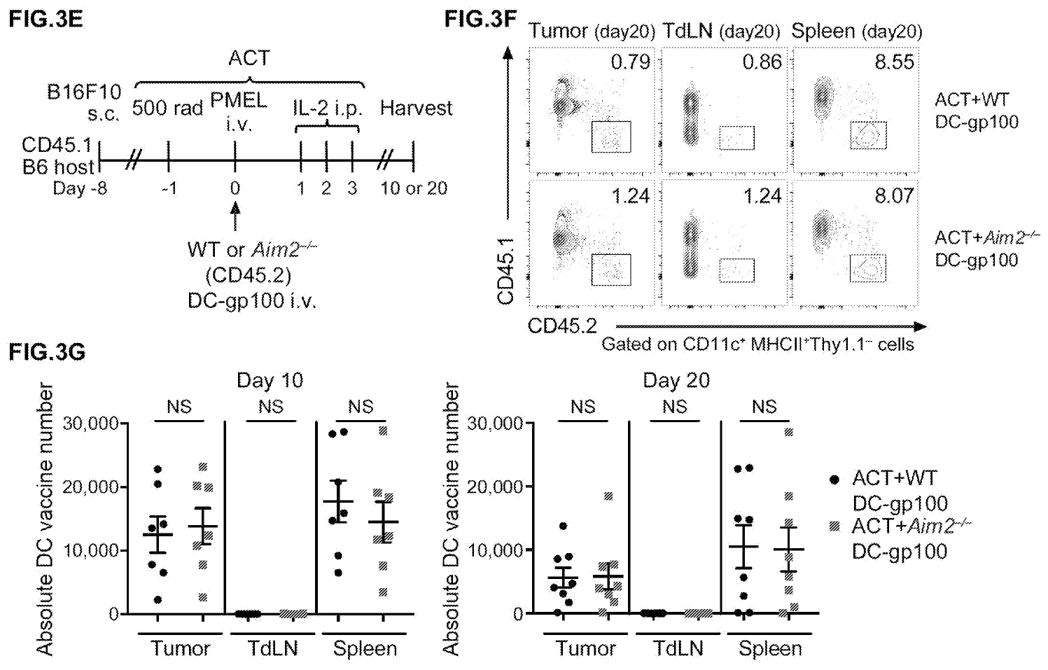

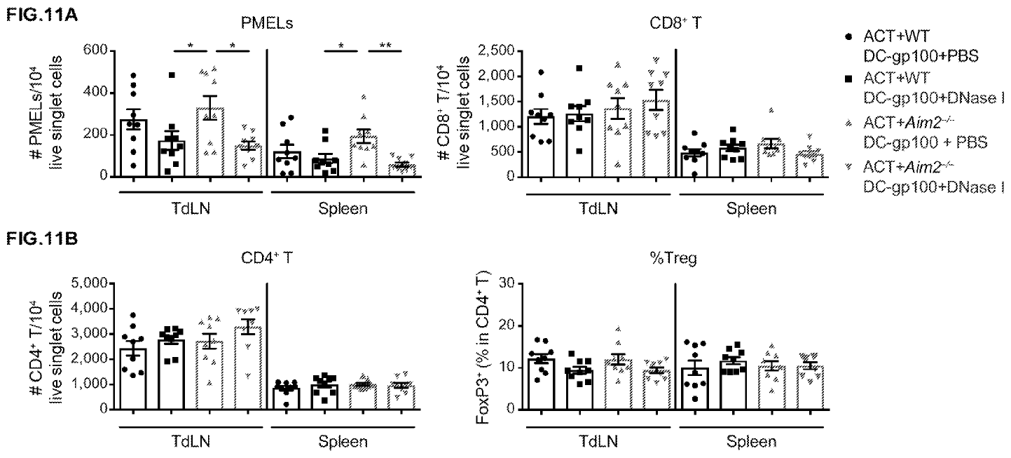

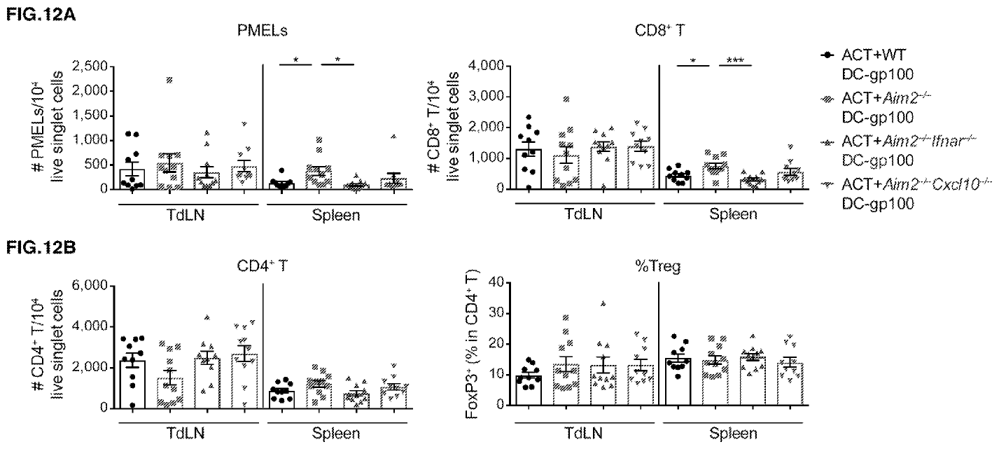

To determine whether enhanced anti-melanoma immunity of Aim2^{-/-} DC vaccine depends on the recognition of tumor-derived DNA, we performed ACT with DC vaccination while injecting the tumor with DNase I (FIG. 3A). The therapeutic effect of Aim2^{-/-} DC-gp100 on ACT was abrogated in mice intratumorally administered DNase I (FIG. 3B). Tumors injected with DNase I contained fewer PMELs, CD8⁺ T cells, a higher proportion of Tregs, and a smaller PMEL/Treg ratio than tumors injected with PBS (FIGS. 3C and 3D). Furthermore, intratumoral DNase I treatment significantly decreased the numbers of PMELs in TdLN and spleen, whereas total CD8⁺, CD4⁺ T cells, and the proportion of Tregs were unchanged (FIGS. 11A and 11B). These results suggested that the enhanced anti-tumor immunity of the Aim2^{-/-} DC vaccine depends on the recognition of tumor-derived DNA.

AIM2 senses the presence of cytosolic DNA and thereby can induce pyroptosis of the cell. We sought to determine whether suppression of pyroptosis was required for the enhanced antitumor immunity by Aim2^{-/-} DC vaccine in vivo. To test this, we performed ACT with DC vaccination using WT or Aim2^{-/-} BMDCs into CD45.1 hosts and quantified the vaccinated DCs infiltrating the tumor at 10 and 20 days after PMEL transfer (FIG. 3E-3G). Similar to the tumor analyzed at 1.5 days after PMEL transfer (FIGS. 10B and 10C), there was no difference in the number of vaccinated DCs infiltrating the tumor, TdLN, or spleen. These results suggest that the enhanced anti-tumor immunity of the Aim2^{-/-} DC vaccine does not depend on suppression of pyroptosis.

### AIM2-Deficient DC Vaccination Requires Autologous Type I IFN Signaling and Promotes Tumor Antigen-Specific CD8+ T Cell Infiltration into the Tumor via CXCL10

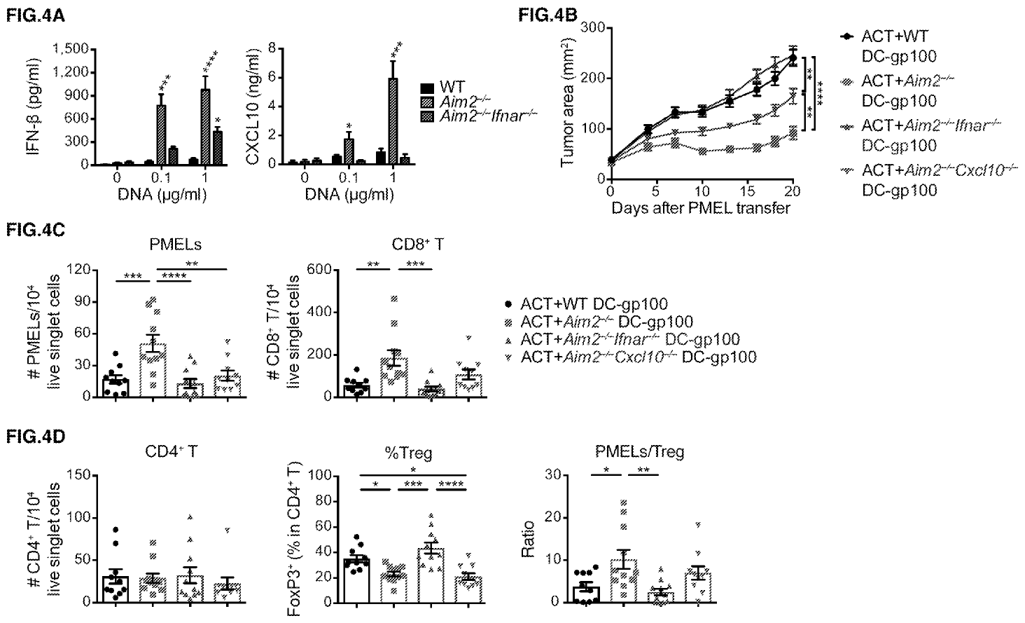

As shown earlier, Aim2^{-/-} BMDCs produce greater amounts of IFN-β and CXCL10 compared to WT BMDCs following in vitro stimulation with tumor DNA. This enhanced cytokine production in Aim2^{-/-} BMDCs was dependent on type I IFN signaling, since these responses were impaired in Aim2 ^{-/-}Ifnar^{-/-} BMDCs (FIG. 4A). These results suggest that autocrine type I IFN signaling in BMDCs is required for the enhanced inflammatory function of the Aim2^{-/-} DC vaccine.

We next sought to determine whether autologous type I IFN signaling and CXCL10 production were required for enhanced antitumor immunity by the Aim2^{-/-} DC vaccine in vivo. To test this, we performed ACT with DC vaccination using Aim2^{-/-}Ifnar^{-/-} or Aim2^{-/-}Cxcl10^{-/-} BMDCs. Vaccination with Aim2^{-/-}Ifnar^{-/-} DC-gp100 eliminated the enhanced antitumor effect of Aim2^{-/-} DC vaccination, such that hosts receiving Aim2^{-/-}Ifnar^{-/-} DC-gp100 experienced similar tumor growth as those receiving WT DC-gp100. Similarly, but to a lesser extent, Aim2^{-/-}Cxcl10^{-/-} DC-gp100 revealed a decreased antitumor effect (FIG. 4B). Within the tumor, hosts receiving Aim2^{-/-} DC-gp100 had significantly higher numbers of PMELs than other groups and significantly higher numbers of total CD8⁺ T cells than hosts receiving WT and Aim2^{-/-}Ifnar^{-/-} DC-gp100, whereas the was no difference in numbers of CD4⁺ T cells among all groups (FIGS. 4C and 4D). In contrast, hosts receiving Aim2^{-/-} DC-gp100 and Aim2^{-/-}Cxcl10^{-/-} DC-gp100 showed a significantly lower proportion of Tregs compared to those receiving WT and Aim2^{-/-}Ifnar^{-/-} DC-gp100. In addition, the PMEL/Treg ratio was significantly higher in hosts receiving Aim2^{-/-} DC-gp100 compared to those receiving WT and Aim2^{-/-}Ifnar^{-/-} DC-gp100 (FIG. 4D). Within the spleen, hosts receiving Aim2^{-/-} DC-gp100 showed significantly higher numbers of PMELs and total CD8⁺ T cells than those receiving WT and Aim2^{-/-}Ifnar^{-/-} DC-gp100, whereas there was no difference among all groups in TdLN (FIG. 11A). Moreover, there was no difference in the numbers of CD4⁺ T cells and proportion of Tregs in TdLN and spleen among all groups (FIGS. 11A and 11B). These results suggest that Aim2^{-/-} DCs during vaccination represent the primary type I IFN-sensing cells and that intravenous injection of the Aim2^{-/-} DC vaccine promotes the migration of antigen-specific CD8⁺ T cells into the tumor via CXCL10. In addition, tumor-infiltrating Aim2^{-/-} DCs decrease Treg migration to the tumor through type I IFN signaling, but not via CXCL10.

### AIM2 is Required for IL-1β and IL-18 Production, which Promote Melanoma Treg Accumulation and Tumor Growth in Vivo

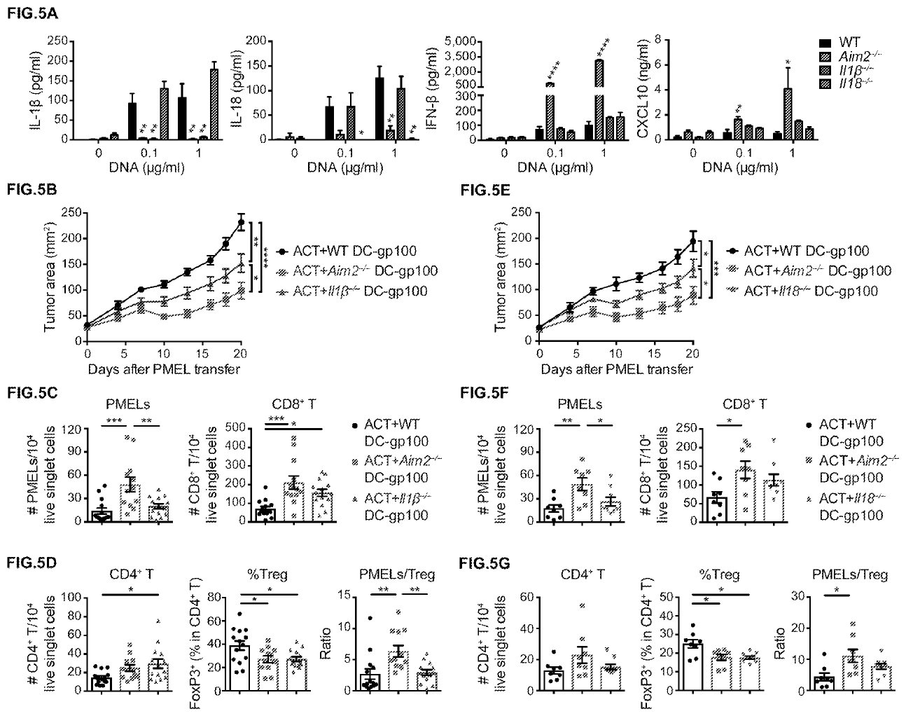

Consistent with the well-established role of AIM2 as a caspase-1 activating inflammasome (Rathinam et al., 2010), AIM2 was required for the secretion of IL-1β and IL-18 from BMDCs in response to stimulation with tumor-derived DNA. The dsDNA-induced IFN-β or CXCL10 production was normal in BMDC lacking IL-1β or IL-18 as expected, suggesting that neither IL-1β nor IL-18 deficiency recapitulates the enhanced effect on the STING pathway seen with AIM2^{-/-} BMDCs (FIG. 5A). We next assessed whether there is an enhanced antitumor effect of DC vaccination by performing ACT with Il1b^{-/-} or Il18^{-/-} BMDCs.

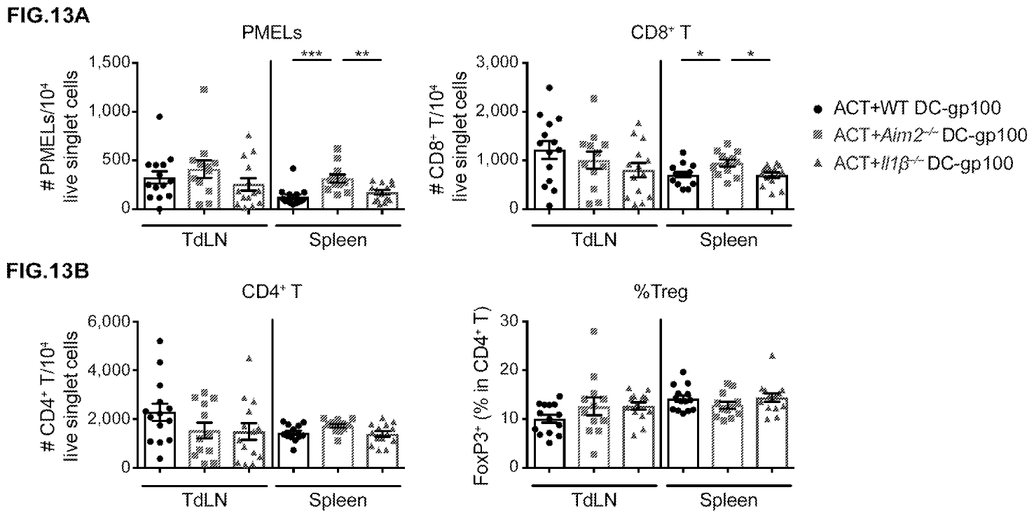

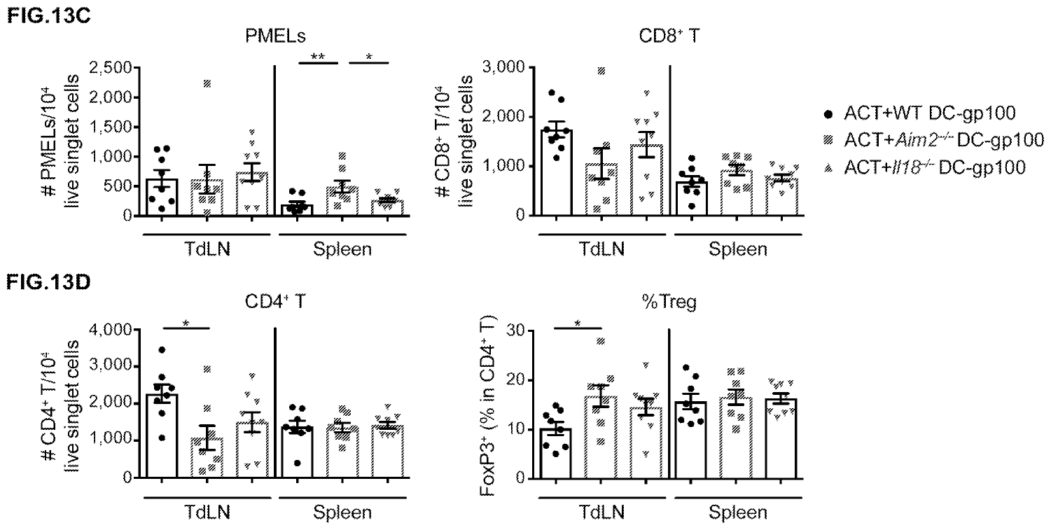

In hosts receiving WT, Aim2^{-/-}, or Il1b^{-/-} DC-gp100, the tumor burden of those receiving Il1b^{-/-} DC-gp100 was intermediate between those receiving WT DC-gp100 and those receiving Aim2^{-/-} DC-gp100 (FIG. 5B). Within the tumor, hosts receiving Aim2^{-/-} DC-gp100 had significantly greater number of PMELs and higher PMEL/Treg ratio than the other groups and hosts receiving Aim2^{-/-} DC-gp100 showed a significantly higher PMEL/Treg ratio than hosts receiving WT DC-gp100 but not Il1b^{-/-} DC-gp100 (FIGS. 5C and 5D). In contrast, hosts receiving Aim2^{-/-} and Il1b^{-/-} DC-gp100 showed a significantly lower proportion of Tregs than hosts receiving WT DC-gp100, and hosts with Il1b^{-/-} DC-gp100 also had significantly higher numbers of CD4⁺ T cells than other groups (FIG. 5D). Within the spleen, hosts receiving Aim2^{-/-} DC-gp100 showed significantly higher numbers of PMELs and total CD8⁺ T cells than other groups, whereas there was no difference among all groups in TdLN (FIG. 13A). The numbers of CD4⁺ T cells and proportion of Tregs in TdLN and spleen were similar among all groups (FIG. 13B). Together, these data suggest that reduced production of IL-1β in Aim2^{-/-} DC vaccine prevents Treg tumor infiltration and promotes anti-tumor immune responses, but this does not fully recapitulate the antitumor effect of AIM2-deficiency.

Similarly, in ACT with WT, Aim2^{-/-}, or Il18^{-/-} DC-gp100, the tumor burden of hosts receiving Il18^{-/-} DC-gp100 was intermediate between those receiving WT DC-gp100 and those receiving Aim2^{-/-} DC-gp100 (FIG. 5E). Within the tumor, hosts receiving Aim2^{-/-} DC-gp100 had significantly greater numbers of PMELs and total CD8⁺ T cells than other groups and hosts receiving Aim2^{-/-} DC-gp100 showed a significantly higher PMEL/Treg ratio than hosts receiving WT DC-gp100 but not Il18^{-/-} DC-gp100 (FIGS. 5F and 5G). In contrast, hosts receiving Aim2^{-/-} DC-gp100 and Il18^{-/-} DC-gp100 showed a significantly lower proportion of Tregs than hosts receiving WT DC-gp100, and there was no difference in the numbers of CD4⁺ T cells among all groups (FIG. 5G). Within the spleen, hosts receiving Aim2^{-/-} DC-gp100 showed significantly higher numbers of PMELs compared to other groups, whereas there was no difference among all groups in TdLN. There was also no difference in CD8⁺ T cell numbers in TdLN and spleen among all groups (FIG. 13C). In contrast, hosts receiving Aim2^{-/-} DC-gp100 showed significantly lower CD4⁺ T cell numbers and higher proportion of Tregs in the TdLN than those receiving WT DC-gp100 whereas there was no difference in spleen among all groups (FIG. 13D). These results suggested that reduced production of IL-18 in Aim2^{-/-} DC vaccine could also prevent Treg tumor infiltration. Taken together, these findings reveal that AIM2 regulates anti-melanoma immunity of tumor-infiltrating DC vaccination both by suppressing the STING-type I IFN pathway and through its effects promoting IL-1β and IL-18 production in response to tumor-derived DNA.

### Silencing Aim2 in Vaccinated DC Enhances the Efficacy of ACT Against Melanoma

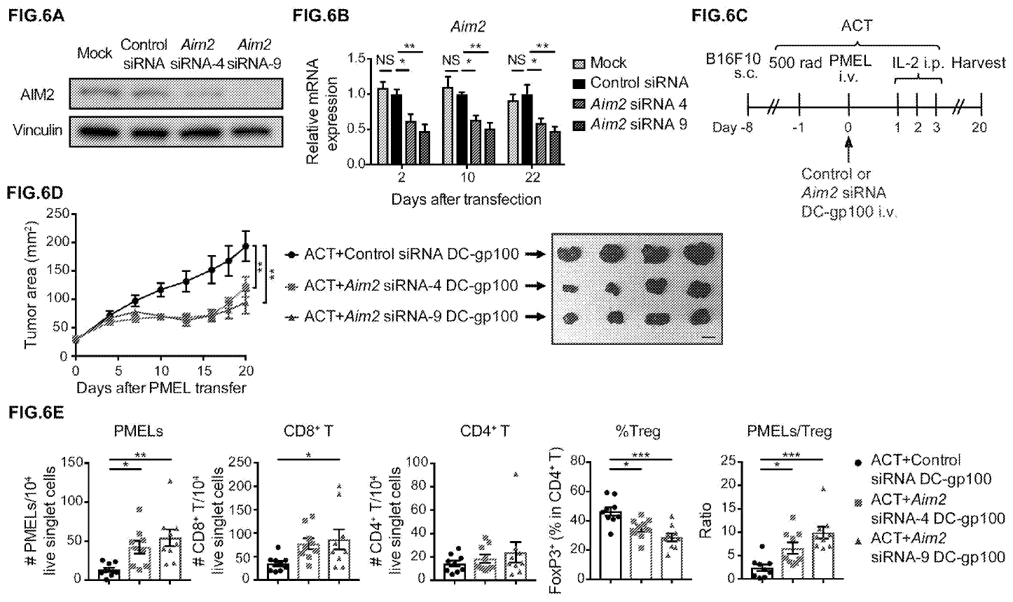

To determine whether targeting AIM2 in the DC vaccine could be used therapeutically, we next evaluated whether silencing Aim2 expression could improve the efficacy of ACT in the setting of WT DC vaccination. Twelve different Aim2 targeted hydrophobically modified, fully chemically stabilized siRNAs that have an ability to maintain sustained silencing with a single treatment were synthesized to develop an AIM2-silenced DC vaccine. Among the twelve Aim2 siRNAs, three showed significant Aim2 gene suppression compared to Mock (transfection reagent only)-transfected BMDCs while others did not (FIG. 13A). Aim2 siRNA 4 and Aim2 siRNA 9, which exhibited the strongest and second strongest Aim2 suppression were selected and used for further experiments. WT BMDCs transfected with Aim2 siRNA (−4 or −9) showed markedly lower mRNA and protein expression of AIM2 than control siRNA-transfected and mock (transfection reagent only)-transfected BMDCs (FIGS. 6A and 6B). Furthermore, we observed that knockdown of Aim2 mRNA persisted for as long as 22 days after transfection (FIG. 6B).

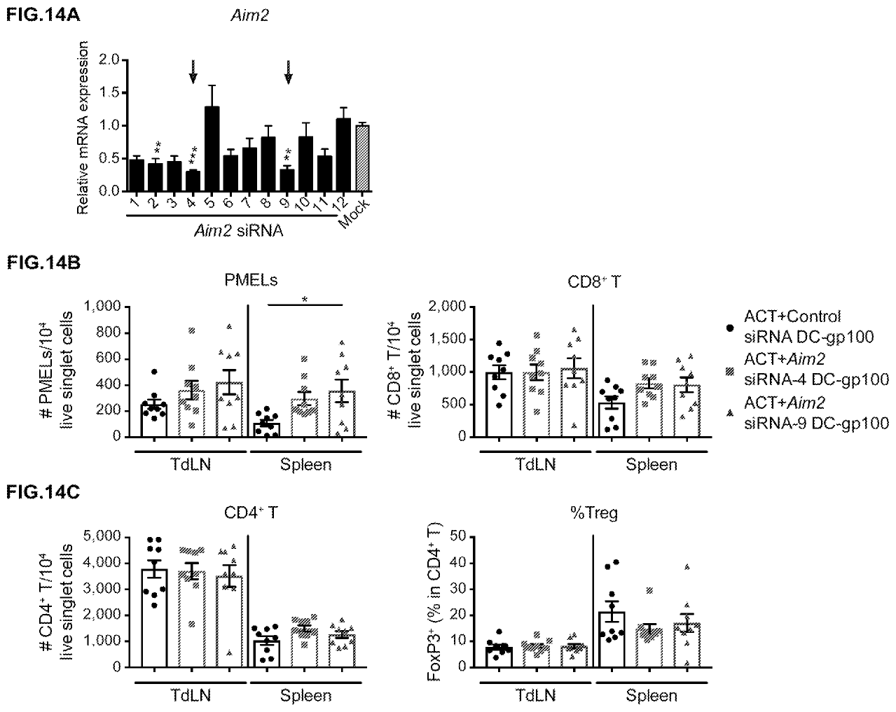

Following ACT in combination with DC-gp100 transfected with either control or Aim2 siRNA (FIG. 6C), we found that the tumor burden of hosts receiving Aim2 siRNA-transfected DC-gp100 was significantly smaller compared to those treated with control siRNA-transfected DC-gp100 (FIG. 6D). The numbers of PMELs, CD8⁺, CD4⁺ T cells, and PMEL/Treg ratio were significantly higher in hosts receiving Aim2 siRNA-transfected DC-gp100 than hosts with control siRNA-transfected DC-gp100 while, in contrast, the proportion of Tregs was significantly lower (FIG. 6E). Furthermore, the numbers of PMELs in the spleen was significantly higher in hosts receiving Aim2 siRNA-transfected DC-gp100 than in hosts with control siRNA-transfected DC-gp100, whereas it did not differ in TdLN. Furthermore, there was no difference in the numbers of CD8⁺, CD4⁺ T cells, and proportion of Tregs in TdLN and spleen (FIG. 6E, FIG. 14A, and FIG. 14B). These results indicate that treatment of WT DCs with Aim2 siRNAs prior to vaccination recapitulates the therapeutic benefit observed with Aim2^{-/-} DC vaccine, providing a therapeutic option relevant to clinical care.

### AIM2-Deficient DC Vaccination Provides Additive Anti-Tumor Effects when Combined With Anti-PD-1 Immunotherapy

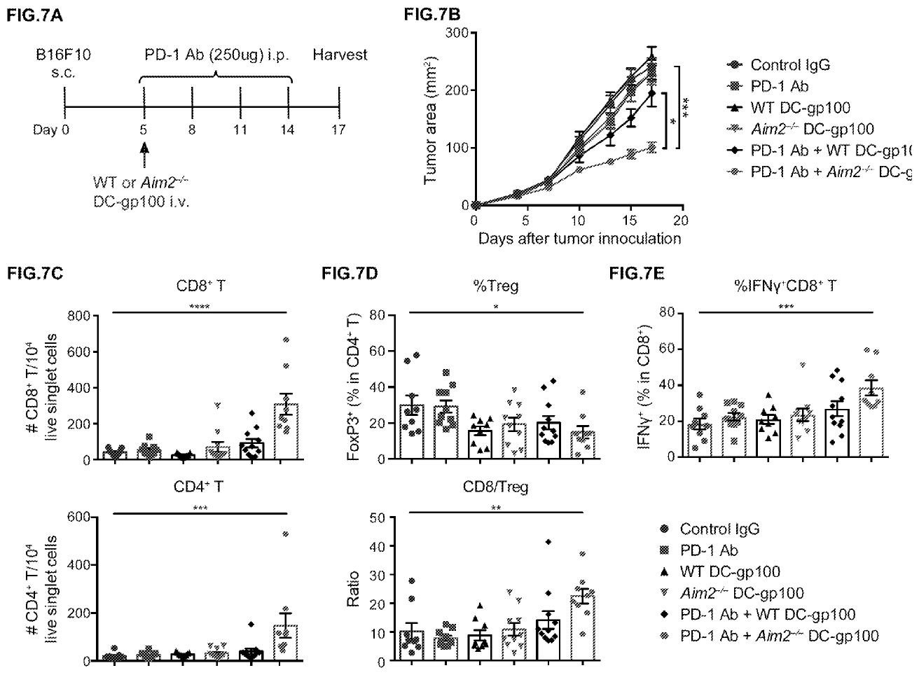

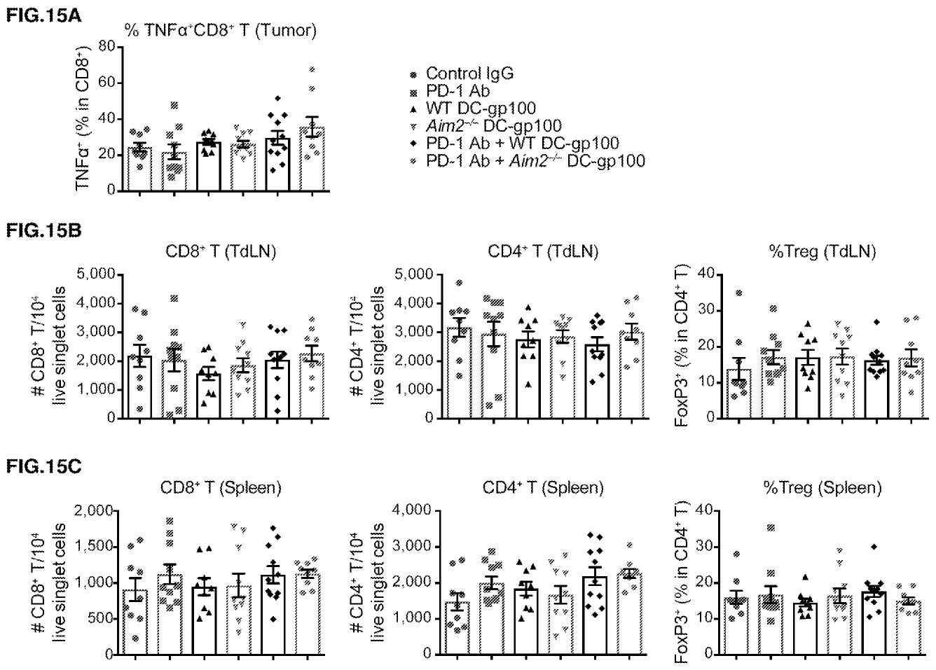

Because the failure of immunotherapy with PD-1 antibody is frequently due to “cold” tumors without sufficient T cell infiltration and following our observation that Aim2^{-/-} DC vaccination enhances tumor infiltration, we assessed whether Aim2^{-/-} DC-gp100 could augment the efficacy of anti-PD-1 immunotherapy in this poorly immunogenic B16F10 melanoma model. To do this, we treated B16F10-bearing WT mice with control IgG, PD-1 Ab, WT DC-gp100, Aim2^{-/-} DC-gp100, PD-1 Ab+WT DC-gp100, or PD-1 Ab+Aim2^{-/-} DC-gp100 (FIG. 7A). Compared with hosts treated with control IgG, only hosts that received PD-1 Ab+Aim2^{-/-} DC-gp100 showed significantly lower tumor burden (FIG. 7B). Notably, the tumor burden of hosts treated with Aim2^{-/-} DC-gp100 was similar to that of hosts treated with WT DC-gp100, unlike previous experiments in which we used radiation as part of ACT, whereas hosts treated with PD-1 Ab+Aim2^{-/-} DC-gp100 showed significantly lower tumor burden than hosts treated with PD-1 Ab+WT DC-gp100 (FIG. 7B). These results imply that intravenous injection of Aim2^{-/-} DC vaccine without radiation does not provide enough release of tumor-derived DNA by itself and requires co-treatment such as ACT or PD-1 Ab to enhance anti-melanoma immunity. Consistent with the result of tumor growth, hosts receiving PD-1 Ab+Aim2^{-/-} DC-gp100 were the only ones that showed significantly greater numbers of CD8⁺ and CD4⁺ T cells, and higher CD8/Treg ratio and percentage of IFN-γ producing CD8⁺ T cells, as well as a significantly lower proportion of Tregs compared with the control group (FIG. 7C-7E). In contrast, there were no differences in percentage of TNF-α producing CD8⁺ T cells in the tumor, or numbers of total CD8⁺ T cells, CD4⁺ T cells, proportion of Tregs, and CD8/Treg ratio in the TdLNs or spleen among all treatments (FIG. 7E and FIG. 15A-15C). Thus, these results demonstrate that Aim2^{-/-} DC vaccination not only provides additive anti-melanoma immunity to ACT but also to anti-PD-1 immunotherapy.

### siRNA Targeting of AIM2 in Human Monocyte-Derived DCs Results in Enhanced Responses to Tumor-Derived DNA

Finally, we addressed whether the therapeutic implications in our mouse model could be extended to the human system. First, we confirmed that AIM2 protein is expressed in mature human monocyte-derived DCs (MoDCs), a DC subset that is frequently used for DC vaccines in clinical trials for cancers. In addition, this expression could be effectively silenced by AIM2 siRNA (−2 or −4) (FIG. 8A). We found that priming with LPS to convert immature MODCs to mature MoDCs induces AIM2 expression further (FIG. 8B). Next, we tested whether AIM2 in mature MoDCs inhibits the activation of STING-type I IFN signaling and promotes the secretion of IL-1β and IL-18 in response to cytosolic tumor-derived DNA as we observed in mouse BMDCs. We stimulated LPS-primed MoDCs with human melanoma xenograft-derived DNA (melanoma DNA), delivered via lipofection. IRF3 is a protein activated by STING-type I IFN signaling. We found that LPS priming of MoDCs induced pTBK1 regardless of stimulation by melanoma DNA and AIM2 siRNA-transfected MoDCs showed similar level of pTKB1 in response to exposure to melanoma DNA compared to control siRNA-transfected MoDCs. In contrast, AIM2 siRNA-transfected MoDCs showed enhanced phosphorylation of IRF (pIRF3) in response to exposure to melanoma DNA compared to control siRNA-transfected MoDCs (FIG. 8C). Furthermore, AIM2 siRNA-transfected and control siRNA-transfected MoDCs induced IFN-β, CXCL10, IL-1β, and IL-18 production following stimulation with melanoma DNA and AIM2 siRNA-transfected MoDCs secreted significantly more IFN-β and CXCL10 (FIG. 8D) but significantly less IL-1β and IL-18 than control siRNA-transfected MoDCs (FIG. 8E). These results imply that AIM2 in human mature MoDCs responds in a similar way to mouse BMDCs and thus can be used to generate a DC vaccine to improve the efficacy of melanoma immunotherapy in patients.

The data in this Example support using vaccination with Aim2^{-/-} DCs as an adjuvant to ACT therapy or treatment with PD-1 antibodies.

### REFERENCES

Anz, D., Rapp, M., Eiber, S., Koelzer, V. H., Thaler, R., Haubner, S., Knott, M., Nagel, S., Golic, M., Wiedemann, G. M., et al. (2015). Suppression of intratumoral CCL22 by type i interferon inhibits migration of regulatory T cells and blocks cancer progression. Cancer Res 75, 4483-4493.

Bald, T., Landsberg, J., Lopez-Ramos, D., Renn, M., Glodde, N., Jansen, P., Gaffal, E., Steitz, J., Tolba, R., Kalinke, U., et al. (2014). Immune cell-poor melanomas benefit from PD-1 blockade after targeted type I IFN activation. Cancer Discov 4, 674-687.

Brinster, C., and Shevach, E. M. (2008). Costimulatory effects of IL-1 on the expansion/differentiation of CD4+CD25+Foxp3+ and CD4+CD25+Foxp3− T cells. J Leukoc Biol 84, 480-487.

Chen, P. L., Roh, W., Reuben, A., Cooper, Z. A., Spencer, C. N., Prieto, P. A., Miller, J. P., Bassett, R. L., Gopalakrishnan, V., Wani, K., et al. (2016). Analysis of Immune Signatures in Longitudinal Tumor Samples Yields Insight into Biomarkers of Response and Mechanisms of Resistance to Immune Checkpoint Blockade. Cancer Discov 6, 827-837.

Corrales, L., Glickman, L. H., McWhirter, S. M., Kanne, D. B., Sivick, K. E., Katibah, G. E., Woo, S. R., Lemmens, E., Banda, T., Leong, J. J., et al. (2015). Direct Activation of STING in the Tumor Microenvironment Leads to Potent and Systemic Tumor Regression and Immunity. Cell Rep 11, 1018-1030.

Corrales, L., Matson, V., Flood, B., Spranger, S., and Gajewski, T. F. (2017). Innate immune signaling and regulation in cancer immunotherapy. Cell Res 27, 96-108.

Corrales, L., Woo, S. R., Williams, J. B., McWhirter, S. M., Dubensky, T. W., Jr., and Gajewski, T. F. (2016). Antagonism of the STING Pathway via Activation of the AIM2 Inflammasome by Intracellular DNA. J Immunol 196, 3191-3198.

Deng, L., Liang, H., Xu, M., Yang, X., Burnette, B., Arina, A., Li, X. D., Mauceri, H., Beckett, M., Darga, T., et al. (2014). STING-Dependent Cytosolic DNA Sensing Promotes Radiation-Induced Type I Interferon-Dependent Antitumor Immunity in Immunogenic Tumors, Immunity 41, 843-852.

Fabbi, M., Carboni, G., and Ferrini, S. (2015). Context-dependent role of IL-18 in cancer biology and counter-regulation by IL-18BP. J Leukoc Biol 97, 665-675.

Gehrke, S., Otsuka, A., Huber, R., Meier, B., Kistowska, M., Fenini, G., Cheng, P., Dummer, R., Kerl, K., Contassot, E., and French, L. E. (2014). Metastatic melanoma cell lines do not secrete IL-1beta but promote IL-1beta production from macrophages. J Dermatol Sci 74, 167-169.

Goff, S. L., Dudley, M. E., Citrin, D. E., Somerville, R. P., Wunderlich, J. R., Danforth, D. N., Zlott, D. A., Yang, J. C., Sherry, R. M., Kammula, U. S., et al. (2016). Randomized, Prospective Evaluation Comparing Intensity of Lymphodepletion Before Adoptive Transfer of Tumor-Infiltrating Lymphocytes for Patients With Metastatic Melanoma. J Clin Oncol 34, 2389-2397.

Hassler, M. R., Turanov, A. A., Alterman, J. F., Haraszti, R. A., Coles, A. H., Osborn, M. F., Echeverria, D., Nikan, M., Salomon, W. E., Roux, L., et al. (2018). Comparison of partially and fully chemically-modified siRNA in conjugate-mediated delivery in vivo. Nucleic Acids Res 46, 2185-2196.

Helft, J., Bottcher, J., Chakravarty, P., Zelenay, S., Huotari, J., Schraml, B. U., Goubau, D., and Reis e Sousa, C. (2015). GM-CSF Mouse Bone Marrow Cultures Comprise a Heterogeneous Population of CD11c(+)MHCII(+) Macrophages and Dendritic Cells. Immunity 42, 1197-1211.

Homet Moreno, B., Zaretsky, J. M., Garcia-Diaz, A., Tsoi, J., Parisi, G., Robert, L., Meeth, K., Ndoye, A., Bosenberg, M., Weeraratna, A. T., et al. (2016). Response to Programmed Cell Death-1 Blockade in a Murine Melanoma Syngeneic Model Requires Costimulation, CD4, and CD8 T Cells. Cancer Immunol Res 4, 845-857.

Horai, R., Asano, M., Sudo, K., Kanuka, H., Suzuki, M., Nishihara, M., Takahashi, M., and Iwakura, Y. (1998). Production of mice deficient in genes for interleukin (IL)-1alpha, IL-1beta, IL-1alpha/beta, and IL-1 receptor antagonist shows that IL-1beta is crucial in turpentine-induced fever development and glucocorticoid secretion. J Exp Med 187, 1463-1475.

Hou, Y., Liang, H., Rao, E., Zheng, W., Huang, X., Deng, L., Zhang, Y., Yu, X., Xu, M., Mauceri, H., et al. (2018). Non-canonical NF-kappaB Antagonizes STING Sensor-Mediated DNA Sensing in Radiotherapy. Immunity 49, 490-503.e494.

Ishikawa, H., and Barber, G. N. (2008). STING is an endoplasmic reticulum adaptor that facilitates innate immune signalling. Nature 455, 674-678.

Jones, J. W., Kayagaki, N., Broz, P., Henry, T., Newton, K., O'Rourke, K., Chan, S., Dong, J., Qu, Y., Roose-Girma, M., et al. (2010). Absent in melanoma 2 is required for innate immune recognition of Francisella tularensis. Proc Natl Acad of Sci USA 107, 9771-9776.

Kakizaki, A., Fujimura, T., Furudate, S., Kambayashi, Y., Yamauchi, T., Yagita, H., and Aiba, S. (2015). Immunomodulatory effect of peritumorally administered interferon-beta on melanoma through tumor-associated macrophages. Oncoimmunology 4, e1047584.

Kaplanski, G. (2018). Interleukin-18: Biological properties and role in disease pathogenesis. Immunol Rev 281, 138-153.

Krzastek, S. C., Goliadze, E., Zhou, S., Petrossian, A., Youniss, F., Sundaresan, G., Wang, L., Zweit, J., and Guruli, G. (2018). Dendritic cell trafficking in tumor-bearing mice. Cancer Immunol Immunother

Li, K., Qu, S., Chen, X., Wu, Q., and Shi, M. (2017). Promising Targets for Cancer Immunotherapy: TLRs, RLRs, and STING-Mediated Innate Immune Pathways. Int J Mol Sci 18.

Lim, H. X., Hong, H. J., Cho, D., and Kim, T. S. (2014). IL-18 enhances immunosuppressive responses by promoting differentiation into monocytic myeloid-derived suppressor cells. J Immunol 193, 5453-5460.

Lou, Y., Wang, G., Lizee, G., Kim, G. J., Finkelstein, S. E., Feng, C., Restifo. N. P., and Hwu, P. (2004). Dendritic cells strongly boost the antitumor activity of adoptively transferred T cells in vivo. Cancer Res 64, 6783-6790.

Lucarini, V., Buccione, C., Ziccheddu, G., Peschiaroli, F., Sestili, P., Puglisi, R., Mattia, G., Zanetti, C., Parolini, I., Bracci, L., et al. (2017). Combining Type I Interferons and 5-Aza-2′-Deoxycitidine to Improve Anti-Tumor Response against Melanoma. J Invest Dermatol 137, 159-169.

Lutz, M. B., Kukutsch, N., Ogilvie, A. L., Rossner, S., Koch, F., Romani, N., and Schuler, G. (1999). An advanced culture method for generating large quantities of highly pure dendritic cells from mouse bone marrow. J Immunol Methods 223, 77-92.

Ma, Z., Li, W., Yoshiya, S., Xu, Y., Hata, M., El-Darawish, Y., Markova, T., Yamanishi, K., Yamanishi, H., Tahara, H., et al. (2016). Augmentation of Immune Checkpoint Cancer Immunotherapy with IL18. Clin Cancer Res 22, 2969-2980.

Man, S. M., Karki, R., and Kanneganti, T. D. (2016). AIM2 inflammasome in infection, cancer, and autoimmunity: Role in DNA sensing, inflammation, and innate immunity. Eur Jour Immunol 46, 269-280.

McCauley S. M., Kim K., Nowosielska A., Dauphin A., Yurkovetskiy L., Diehl W. E., and Luban J. (2018). Intron-containing RNA from the HIV-1 provirus activates type I interferon and inflammatory cytokines. Nat Commun 9, 5305.

Meeth, K., Wang, J. X., Micevic, G., Damsky, W., and Bosenberg, M. W. (2016). The YUMM lines: a series of congenic mouse melanoma cell lines with defined genetic alterations. Pigment Cell Melanoma Res 29, 590-597.

Mullins, D. W., Sheasley, S. L., Ream, R. M., Bullock, T. N., Fu, Y. X., and Engelhard, V. H. (2003). Route of immunization with peptide-pulsed dendritic cells controls the distribution of memory and effector T cells in lymphoid tissues and determines the pattern of regional tumor control. J Exp Med 198, 1023-1034.

Oertli, M., Sundquist, M., Hitzler, I., Engler, D. B., Arnold, I. C., Reuter, S., Maxeiner, J., Hansson, M., Taube, C., Quiding-Jarbrink, M., and Muller, A. (2012). DC-derived IL-18 drives Treg differentiation, murine Helicobacter pylon-specific immune tolerance, and asthma protection. J Clin Invest 122, 1082-1096.

Pan, W., Zhu, S., Qu, K., Meeth, K., Cheng, J., He, K., Ma, H., Liao, Y., Wen, X., Roden, C., et al. (2017). The DNA Methylcytosine Dioxygenase Tet2 Sustains Immunosuppressive Function of Tumor-Infiltrating Myeloid Cells to Promote Melanoma Progression. Immunity 47, 284-297.e285.

Peng, W., Chen, J. Q., Liu, C., Malu, S., Creasy, C., Tetzlaff, M. T., Xu, C., McKenzie, J. A., Zhang, C., Liang, X., et al. (2016). Loss of PTEN Promotes Resistance to T Cell-Mediated Immunotherapy. Cancer Discov 6, 202-216.

Qin, Y., Ekmekcioglu, S., Liu, P., Duncan, L. M., Lizee, G., Poindexter, N., and Grimm, E. A. (2011). Constitutive aberrant endogenous interleukin-1 facilitates inflammation and growth in human melanoma. Mol Cancer Research 9, 1537-1550.

Rashighi, M., Agarwal, P., Richmond, J. M., Harris, T. H., Dresser, K., Su, M. W., Zhou, Y., Deng, A., Hunter, C. A., Luster, A. D., and Harris, J. E. (2014). CXCL10 is critical for the progression and maintenance of depigmentation in a mouse model of vitiligo. Sci Transl Med 6, 223ra223.

Ribas, A., Hamid, O., Daud, A., Hodi, F. S., Wolchok, J. D., Kefford, R., Joshua, A. M., Patnaik, A., Hwu, W. J., Weber, J. S., et al. (2016). Association of Pembrolizumab With Tumor Response and Survival Among Patients With Advanced Melanoma. Jama 315, 1600-1609.

Robert, C., Schachter, J., Long, G. V., Arance, A., Grob, J. J., Mortier, L., Daud, A., Carlino, M. S., McNeil, C., Lotem, M., et al. (2015). Pembrolizumab versus Ipilimumab in Advanced Melanoma. N Engl J Med 372, 2521-2532.

Spranger, S., Bao, R., and Gajewski, T. F. (2015). Melanoma-intrinsic beta-catenin signalling prevents anti-tumour immunity. Nature 523, 231-235.

Spranger, S., Dai, D., Horton, B., and Gajewski, T. F. (2017). Tumor-Residing Batf3 Dendritic Cells Are Required for Effector T Cell Trafficking and Adoptive T Cell Therapy. Cancer Cell 31, 711-723.e714.

Tumeh, P. C., Harview, C. L., Yearley, J. H., Shintaku, I. P., Taylor, E. J., Robert, L., Chmielowski, B., Spasic, M., Henry, G., Ciobanu, V., et al. (2014). PD-1 blockade induces responses by inhibiting adaptive immune resistance. Nature 515, 568-571.

Woo, S. R., Fuertes, M. B., Corrales, L., Spranger, S., Furdyna, M. J., Leung, M. Y., Duggan, R., Wang, Y., Barber, G. N., Fitzgerald, K. A., et al. (2014). STING-dependent cytosolic DNA sensing mediates innate immune recognition of immunogenic tumors. Immunity 41, 830-842.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A double stranded RNA molecule that is between 15 and 35 bases in length, comprising a sense strand and an antisense strand, wherein the antisense strand comprises a region of complementarity that is substantially complementary to a nucleic acid sequence comprising nucleotides 362-380 of SEQ ID NO:46, nucleotides 662-681 of SEQ ID NO:48, nucleotides 714-732 of SEQ ID NO:46, nucleotides 1034-1051 of SEQ ID NO:48, or nucleotides 941-960 of SEQ ID NO: 48, optionally wherein the RNA molecule is modified.

2. The RNA molecule of claim 1, wherein the sense strand comprises the sequence UUUGUAAAAGUUUUA (SEQ ID NO:29), GUUGAAUUAUAUGCA (SEQ ID NO:27), or GCUGAAAGCUAUAAA (SEQ ID NO:31), or differs by 1, 2, or 3 nucleotides.

3. The RNA molecule of claim 1, wherein the antisense strand comprises the sequence UAAAACUUUUACAAAGAAGA (SEQ ID NO:30), UGCAUAUAAUUCAACUUCUG (SEQ ID NO:28), UUUAUAGCUUUCAGCACCGU (SEQ ID NO:32), or differs by 1, 2, or 3 nucleotides.

4. The RNA molecule of claim 1, wherein the sense strand comprises the sequence (mU)#(mU)#(fU)(mG)(fU)(mA)(fA)(mA)(fA)(mG)(mU)(mU)(fU)#(mU)#(mA)-TegChol (SEQ ID NO:7), (mG)#(mU)#(fU)(mG)(fA)(mA)(fU)(mU)(fA)(mU)(mA)(mU)(fG)#(mC)#(mA)-TegChol (SEQ ID NO:3), or (mG)#(mC)#(fU)(mG)(fA)(mA)(fA)(mG)(fC)(mU)(mA)(mU)(fA)#(mA)#(mA)-TegChol (SEQ ID NO:17), wherein m is 2′-O-methyl, f is 2′-fluoro, # is a phosphorothioate bond, P is a 5′-phosphate, TegChol is 3′-Tetraethylene Glycol (Teg) Cholesterol Conjugate, and ( ) is a phosphodiester bond.

5. The RNA molecule of claim 1, wherein the antisense strand comprises P(mU)#(fA)#(mA)(fA)(fA)(fC)(mU)(fU)(mU)(fU)(mA)(fC)(mA)#(fA)#(mA)#(fG)#(mA)#(mA) #(mG)#(fA) (SEQ ID NO:8), P(mU)#(fG)#(mC)(fA)(fU)(fA)(mU)(fA)(mA)(fU)(mU)(fC)(mA)#(fA)#(mC)#(fU)#(mU)#(mC)#(mU)#(fG) (SEQ ID NO:4), or P(mU)#(fU)#(mU)(fA)(fU)(fA)(mG)(fC)(mU)(fU)(mU)(fC)(mA)#(fG)#(mC)#(fA)#(mC)#(mC)#(mG)#(fU) (SEQ ID NO:18), wherein m is 2′-O-methyl, f is 2′-fluoro, # is a phosphorothioate bond, P is a 5′-phosphate, TegChol is 3′-Tetraethylene Glycol (Teg) Cholesterol Conjugate, and ( ) is a phosphodiester bond.

6. The RNA molecule of claim 1, wherein the sense strand comprises the sequence UUUGUAAAAGUUUUA (SEQ ID NO:29), or differs by 1, 2, or 3, nucleotides, and the antisense strand comprises the sequence UAAAACUUUUACAAAGAAGA (SEQ ID NO:30), or differs by 1, 2, or 3 nucleotides.

7. The RNA molecule of claim 1, wherein the sense strand comprises the sequence (mU)#(mU)#(fU)(mG)(fU)(mA)(fA)(mA)(fA)(mG)(mU)(mU)(fU)#(mU)#(mA)-TegChol (SEQ ID NO:7) and the antisense strand comprises the sequence P(mU)#(fA)#(mA)(fA)(fA)(fC)(mU)(fU)(mU)(fU)(mA)(fC)(mA)#(fA)#(mA)#(fG)#(mA)#(mA)#(mG)#(fA) (SEQ ID NO:8), wherein m is 2′-O-methyl, f is 2′-fluoro, # is a phosphorothioate bond, P is a 5′-phosphate, TegChol is 3′-Tetraethylene Glycol (Teg) Cholesterol Conjugate, and ( ) is a phosphodiester bond.

8. The RNA molecule of claim 1, wherein the sense strand comprises the sequence GUUGAAUUAUAUGCA (SEQ ID NO:27), or differs by 1, 2, or 3, nucleotides, and the antisense strand comprises the sequence UGCAUAUAAUUCAACUUCUG (SEQ ID NO:28), or differs by 1, 2, or 3 nucleotides.

9. The RNA molecule of claim 1, wherein the sense strand comprises the sequence (mG)#(mU)#(fU)(mG)(fA)(mA)(fU)(mU)(fA)(mU)(mA)(mU)(fG)#(mC)#(mA)-TegChol (SEQ ID NO:3) and the antisense strand comprises the sequence P(mU)#(fG)#(mC)(fA)(fU)(fA)(mU)(fA)(mA)(fU)(mU)(fC)(mA)#(fA)#(mC)#(fU)#(mU)#(mC)#(mU)#(fG) (SEQ ID NO:4), wherein m is 2′-O-methyl, f is 2′-fluoro, # is a phosphorothioate bond, P is a 5′-phosphate, TegChol is 3′-Tetraethylene Glycol (Teg) Cholesterol Conjugate, and ( ) is a phosphodiester bond.

10. The RNA molecule of claim 1, wherein the sense strand comprises the sequence GCUGAAAGCUAUAAA (SEQ ID NO:31), or differs by 1, 2, or 3, nucleotides, and the antisense strand comprises the sequence UUUAUAGCUUUCAGCACCGU (SEQ ID NO:32), or differs by 1, 2, or 3 nucleotides.

11. The RNA molecule of claim 1, wherein the sense strand comprises the sequence (mG)#(mC)#(fU)(mG)(fA)(mA)(fA)(mG)(fC)(mU)(mA)(mU)(fA)#(mA)#(mA)-TegChol (SEQ ID NO:17) and the antisense strand comprises the sequence P(mU)#(fU)#(mU)(fA)(fU)(fA)(mG)(fC)(mU)(fU)(mU)(fC)(mA)#(fG)#(mC)#(fA)#(mC)#(mC)#(mG)#(fU) (SEQ ID NO:18), wherein m is 2′-O-methyl, f is 2′-fluoro, # is a phosphorothioate bond, P is a 5′-phosphate, TegChol is 3′-Tetraethylene Glycol (Teg) Cholesterol Conjugate, and ( ) is a phosphodiester bond.

12. A vector comprising: a nucleic acid molecule encoding the RNA of claim 1.

13. A cell comprising the vector of claim 12.

14. The cell of claim 13, which is a dendritic cell.

15. A pharmaceutical composition comprising the RNA molecule of claim 1 and a pharmaceutically acceptable carrier.

16. A method for reducing expression of AIM2 gene in a cell, the method comprising: (a) introducing into the cell the RNA molecule of claim 1, and (b) maintaining the cell produced in step (a) for a time sufficient to obtain degradation of the mRNA transcript of the AIM2 gene, thereby reducing expression of the AIM2 gene in the cell.

17. (canceled)

18. A method of treating cancer in a subject in need thereof, comprising administering to the subject a therapeutically effective amount of the pharmaceutical composition of claim 15.

19.-20. (canceled)

21. The method of claim 18, wherein the method further comprises administering radiation or a cytotoxic agent to the subject.

22. The method of claim 18, wherein the method further comprises administering an immune checkpoint modulator to the subject.

23.-35. (canceled)


SEQUENCE LISTING (48 sequences)

SEQ ID NO: 1 - RNA, 15 nt, Artificial Sequence
  Description of Artificial Sequence Synthetic oligonucleotide
gaaguugaauuauaa

SEQ ID NO: 2 - RNA, 20 nt, Artificial Sequence
  Description of Artificial Sequence Synthetic oligonucleotide
uuauaauucaacuucugaaa

SEQ ID NO: 3 - RNA, 15 nt, Artificial Sequence
  Description of Artificial Sequence Synthetic oligonucleotide
guugaauuauaugca

SEQ ID NO: 4 - RNA, 20 nt, Artificial Sequence
  Description of Artificial Sequence Synthetic oligonucleotide
ugcauauaauucaacuucug

SEQ ID NO: 5 - RNA, 15 nt, Artificial Sequence
  Description of Artificial Sequence Synthetic oligonucleotide
aaacagaugguggca

SEQ ID NO: 6 - RNA, 20 nt, Artificial Sequence
  Description of Artificial Sequence Synthetic oligonucleotide
ugccaccaucuguuucuguu

SEQ ID NO: 7 - RNA, 15 nt, Artificial Sequence
  Description of Artificial Sequence Synthetic oligonucleotide
uuuguaaaaguuuua

SEQ ID NO: 8 - RNA, 20 nt, Artificial Sequence
  Description of Artificial Sequence Synthetic oligonucleotide
uaaaacuuuuacaaagaaga

SEQ ID NO: 9 - RNA, 15 nt, Artificial Sequence
  Description of Artificial Sequence Synthetic oligonucleotide
gaacaauugugaaua

SEQ ID NO: 10 - RNA, 20 nt, Artificial Sequence
  Description of Artificial Sequence Synthetic oligonucleotide
uauucacaauuguuccaagg

SEQ ID NO: 11 - RNA, 15 nt, Artificial Sequence
  Description of Artificial Sequence Synthetic oligonucleotide
acaauugugaaugga

SEQ ID NO: 12 - RNA, 20 nt, Artificial Sequence
  Description of Artificial Sequence Synthetic oligonucleotide
uccauucacaauuguuccaa

SEQ ID NO: 13 - RNA, 15 nt, Artificial Sequence
  Description of Artificial Sequence Synthetic oligonucleotide
gucuaagguuugaga

SEQ ID NO: 14 - RNA, 20 nt, Artificial Sequence
  Description of Artificial Sequence Synthetic oligonucleotide
ucucaaaccuuagacaaagc

SEQ ID NO: 15 - RNA, 15 nt, Artificial Sequence
  Description of Artificial Sequence Synthetic oligonucleotide
ugauaaauaaauaaa

SEQ ID NO: 16 - RNA, 20 nt, Artificial Sequence
  Description of Artificial Sequence Synthetic oligonucleotide
uuuauuuauuuaucaacaau

SEQ ID NO: 17 - RNA, 15 nt, Artificial Sequence
  Description of Artificial Sequence Synthetic oligonucleotide
gcugaaagcuauaaa

SEQ ID NO: 18 - RNA, 20 nt, Artificial Sequence
  Description of Artificial Sequence Synthetic oligonucleotide
uuuauagcuuucagcaccgu

SEQ ID NO: 19 - RNA, 15 nt, Artificial Sequence
  Description of Artificial Sequence Synthetic oligonucleotide
guaaaaguuuuaaaa

SEQ ID NO: 20 - RNA, 20 nt, Artificial Sequence
  Description of Artificial Sequence Synthetic oligonucleotide
uuuuaaaacuuuuacaaaga

SEQ ID NO: 21 - RNA, 15 nt, Artificial Sequence
  Description of Artificial Sequence Synthetic oligonucleotide
uaugcauauugcaaa

SEQ ID NO: 22 - RNA, 20 nt, Artificial Sequence
  Description of Artificial Sequence Synthetic oligonucleotide
uuugcaauaugcauauaauu

SEQ ID NO: 23 - RNA, 15 nt, Artificial Sequence
  Description of Artificial Sequence Synthetic oligonucleotide
uagaaaaggacaaaa

SEQ ID NO: 24 - RNA, 20 nt, Artificial Sequence
  Description of Artificial Sequence Synthetic oligonucleotide
uuuuguccuuuucuaaguaa

SEQ ID NO: 25 - RNA, 15 nt, Artificial Sequence
  Description of Artificial Sequence Synthetic oligonucleotide
ugacaaauacgauua

SEQ ID NO: 26 - RNA, 20 nt, Artificial Sequence
  Description of Artificial Sequence Synthetic oligonucleotide
uaaucguauuugucaaucau

SEQ ID NO: 27 - RNA, 15 nt, Artificial Sequence
  Description of Artificial Sequence Synthetic oligonucleotide
guugaauuauaugca

SEQ ID NO: 28 - RNA, 20 nt, Artificial Sequence
  Description of Artificial Sequence Synthetic oligonucleotide
ugcauauaauucaacuucug

SEQ ID NO: 29 - RNA, 15 nt, Artificial Sequence
  Description of Artificial Sequence Synthetic oligonucleotide
uuuguaaaaguuuua

SEQ ID NO: 30 - RNA, 20 nt, Artificial Sequence
  Description of Artificial Sequence Synthetic oligonucleotide
uaaaacuuuuacaaagaaga

SEQ ID NO: 31 - RNA, 15 nt, Artificial Sequence
  Description of Artificial Sequence Synthetic oligonucleotide
gcugaaagcuauaaa

SEQ ID NO: 32 - RNA, 20 nt, Artificial Sequence
  Description of Artificial Sequence Synthetic oligonucleotide
uuuauagcuuucagcaccgu

SEQ ID NO: 33 - DNA, 20 bp, Artificial Sequence
  Description of Artificial Sequence Synthetic primer
ataagcagctccagctccaa

SEQ ID NO: 34 - DNA, 20 bp, Artificial Sequence
  Description of Artificial Sequence Synthetic primer
ctgtctgctggtggagttca

SEQ ID NO: 35 - DNA, 23 bp, Artificial Sequence
  Description of Artificial Sequence Synthetic primer
atggctaggctctgtgctttcct

SEQ ID NO: 36 - DNA, 24 bp, Artificial Sequence
  Description of Artificial Sequence Synthetic primer
agggctctccagacttctgctctg

SEQ ID NO: 37 - DNA, 20 bp, Artificial Sequence
  Description of Artificial Sequence Synthetic primer
aggggagtgatggagagagg

SEQ ID NO: 38 - DNA, 23 bp, Artificial Sequence
  Description of Artificial Sequence Synthetic primer
tgaaagcgtttagccaaaaaagg

SEQ ID NO: 39 - DNA, 25 bp, Artificial Sequence
  Description of Artificial Sequence Synthetic primer
atctccgttcttcagtgtagcaatg

SEQ ID NO: 40 - DNA, 26 bp, Artificial Sequence
  Description of Artificial Sequence Synthetic primer
acaaatccctcaaagacctcaaacag

SEQ ID NO: 41 - DNA, 22 bp, Artificial Sequence
  Description of Artificial Sequence Synthetic primer
gttgaatctaaccacgaagtcc

SEQ ID NO: 42 - DNA, 23 bp, Artificial Sequence
  Description of Artificial Sequence Synthetic primer
ctacaaggtccagatttcaactg

SEQ ID NO: 43 - DNA, 20 bp, Artificial Sequence
  Description of Artificial Sequence Synthetic primer
ggctgtattcccctccatcg

SEQ ID NO: 44 - DNA, 22 bp, Artificial Sequence
  Description of Artificial Sequence Synthetic primer
ccagttggtaacaatgccatgt

SEQ ID NO: 45 - protein, 343 aa, Homo sapiens
MESKYKEILLLTGLDNITDEELDRFKFFLSDEFNIATGKLHTANRIQVATLMIQNAGAVSAVMKTIRIFQKLNYMLLAKRLQEEKEKVDKQYKSVTKPKPLSQAEMSPAASAAIRNDVAKQRAAPKVSPHVKPEQKQMVAQQESIREGFQKRCLPVMVLKAKKPFTFETQEGKQEMFHATVATEKEFFFVKVFNTLLKDKFIPKRIIIIARYYRHSGFLEVNSASRVLDAESDQKVNVPLNIIRKAGETPKINTLQTQPLGTIVNGLFVVQKVTEKKKNILFDLSDNTGKMEVLGVRNEDTMKCKEGDKVRLTFFTLSKNGEKLQLTSGVHSTIKVIKAKKKT

SEQ ID NO: 46 - DNA, 1394 bp, Homo sapiens
agaagtgtcagagtctttgtagctttgaaagtcacctaggttatttgggcatgctctcctgagtcctctgctagttaagctctctgaaaagaaggtggcagacccggtttgctgatcgccccagggatcaggaggctgatcccaaagttgtcagatggagagtaaatacaaggagatactcttgctaacaggcctggataacatcactgatgaggaactggataggtttaagttctttctttcagacgagtttaatattgccacaggcaaactacatactgcaaacagaatacaagtagctaccttgatgattcaaaatgctggggcggtgtctgcagtgatgaagaccattcgtatttttcagaagttgaattatatgcttttggcaaaacgtcttcaggaggagaaggagaaagttgataagcaatacaaatcggtaacaaaaccaaagccactaagtcaagctgaaatgagtcctgctgcatctgcagccatcagaaatgatgtcgcaaagcaacgtgctgcaccaaaagtctctcctcatgttaagcctgaacagaaacagatggtggcccagcaggaatctatcagagaagggtttcagaagcgctgtttgccagttatggtactgaaagcaaagaagcccttcacgtttgagacccaagaaggcaagcaggagatgtttcatgctacagtggctacagaaaaggaattcttctttgtaaaagtttttaatacactgctgaaagataaattcattccaaagagaataattataatagcaagatattatcggcacagtggtttcttagaggtaaatagcgcctcacgtgtgttagatgctgaatctgaccaaaaggttaatgtcccgctgaacattatcagaaaagctggtgaaaccccgaagatcaacacgcttcaaactcagccccttggaacaattgtgaatggtttgtttgtagtccagaaggtaacagaaaagaagaaaaacatattatttgacctaagtgacaacactgggaaaatggaagtactgggggttagaaacgaggacacaatgaaatgtaaggaaggagataaggttcgacttacattcttcacactgtcaaaaaatggagaaaaactacagctgacatctggagttcatagcaccataaaggttattaaggccaaaaaaaaaacatagagaagtaaaaaggaccaattcaagccaactggtctaagcagcatttaattgaagaatatgtgatacagcctcttcaatcagattgtaagttacctgaaagctgcagttcacaggctcctctctccaccaaattaggatagaataattgctggataaacaaattcagaatatcaacagatgatcacaataaacatctgtttctcattca

SEQ ID NO: 47 - protein, 354 aa, Mus musculus
MESEYREMLLLTGLDHITEEELKRFKYFALTEFQIARSTLDVADRTELADHLIQSAGAASAVTKAINIFQKLNYMHIANALEEKKKEAERKLMTNTKKRGTQKVENRSQAENCSAASATRSDNDFKEQAATEVCPQAKPQKKQMVAEQEAIREDLQKDPLVVTVLKAINPFECETQEGRQEIFHATVATETDFFFVKVLNAQFKDKFIPKRTIKISNYLWHSNFMEVTSSSVVVDVESNHEVPNNVVKRARETPRISKLKIQPCGTIVNGLFKVQKITEEKDRVLYGIHDKTGTMEVLVLGNPSKTKCEEGDKIRLTFFEVSKNGVKIQLKSGPCSFFKVIKAAKPKTDMKSVE

SEQ ID NO: 48 - DNA, 2541 bp, Mus musculus
ttcctgtcctgtctgccgccatgcttccttaactagctgctaggttttttccttgtcgtgatgaaatccaccctcatggacctacactaccgaactggactgctggtatattcatgaagtgcttatgagtggatcgagcagcccctatggattcctgtgaacagaactgctgatttactaacaacgcagatggaagttgcttcaaagaacaacttctgaacaggtattgttgcccattctgtgaataatacaaaggcagtgggaacaagacagtacagaggacttgattcaggagacttgaggtctggccgcatagtcatcctttagaagctgggtggcgtcaggaagttttcctttttctcaatgtaaagtgaagaaaaaaaatccagtgtttctcaactgtactgctattcctatttagctattgtatctaggctgatcctgggactgtgagatggagagtgagtaccgggaaatgctgttgttgaccggcctggaccacatcacggaggaagaactgaaacggttcaagtactttgctttgactgagtttcagattgccaggagcacactcgacgtggcagataggacagagttagctgaccacctgattcaaagtgcaggtgcggcgtctgcagtgaccaaggccattaatattttccagaagttgaattatatgcatattgcaaatgctcttgaagagaaaaagaaagaagctgaacgtaaactcatgaccaatacaaagaagagaggaacacagaaggtagaaaatagaagtcaagctgaaaactgctctgctgcctctgccacccgcagtgacaatgactttaaggaacaggctgctacagaagtctgtcctcaagctaagcctcagaagaaacagatggtggcagaacaggaagccatcagagaagatttacagaaagatccacttgttgtcacggtgctgaaagctataaatccctttgagtgtgagactcaggaaggaagacaagagatatttcatgcaacagtggccacggagacagattttttctttgtaaaagttttaaacgcacagtttaaagataaatttatcccaaagaggacaattaaaatatcaaactacctttggcacagtaacttcatggaggtcaccagttcctcagttgtggttgatgttgaatctaaccacgaagtcccaaataacgttgttaagagagccagggaaactcccaggattagtaaactgaagattcagccatgtggaacaattgtgaatgggctgtttaaagtccagaagataacagaggaaaaagatagagtactgtatggtatacatgataaaacagggacaatggaggtgttggtgctgggaaacccaagcaaaacaaagtgcgaggaaggagacaagattagactcacgttctttgaggtgtcaaaaaatggagtgaaaattcagttgaaatctggaccttgtagcttttttaaggttattaaggctgcaaagccaaaaactgacatgaaaagtgtggagtgaagtcacctcatttgaaaaaccttttcctgaagaatcctgatgctgctccttgaactagactgaactacctgaggatagcattttacaacctcatcatcatattgtattacttagaaaaggacaaatactcaaaaaacatctgaaaaatatatgtaaacttattattaattaagttattaagactgcccaacctggggatccatcctatatacaaccaccaaacccagacactattgcatatgccagcaagattttgctgacaggatcctgatatagctctctcttgtgaggctctgccagtgactgacaagtacagaagcagatgctcacagtcatctattggatggaacacagggcccctaataaaggagctagagaaagtacccaagcagcaagtggtctgcaacgctataggaggaacaacaacatgaactaaccagtaccccccagaactgtgtctccagttgcatatgtagcagaagatggcctggccggtcatcaatgggaggagaggcccttggtcttgcaaagatcatatgccccagtacaggggaatgccagggccaggcagcaggagtggatgtgggtgggttggggagtgtgtgtggggggtgttataggggactttcgggatagcatttgaaatgtaaatgaagaaaatatctaataaaattgttgctttgtctaaggtttgagatatcattcttctctacatagacactgagggtataagtatggcgggattgcagatgtgacagcagggccttgtcggagagacgcctgtgggtgatagagaagattggtgatatataattttttaatttaaaaattttaaatttccttttggggaggaggttacaggtggaggagggtgggtatgatagtactaagaaatcagtgatattggggtatgtgatgtgaaattccctagcactcaataaaagaattatgtttttaaaaagaaagattgttgataaataaataaatatgattttactcatgattcagaaagttagaaaaaa